(19) United States
(12) Patent Application Publication
(10) Pub. No.: US 2007/0037158 A1
(43) Pub. Date: Feb. 15, 2007

(54) Methods of modulating bone growth, bone remodeling and adiposity

(75) Inventors: Edith Gardiner, PADDINGTON, QUEENSLAND (AU); Herbert Herzog, Bondi (AU); Amanda Sainsbury-Salis, Kogarah (AU)

Correspondence Address: QUINE INTELLECTUAL PROPERTY LAW GROUP, P.C., P O BOX 458, ALAMEDA, CA, 94501, US

(73) Assignee: GARVAN INSTITUTE OF MEDICAL RESEARCH

(21) Appl. No.: 10/572,696

(22) PCT Filed: Sep. 18, 2003
(86) PCT No.: PCT/AU03/01227

§ 371 Date: Oct. 5, 2006

(51) Int. Cl. A61K 48/00; C12Q 1/68; G01N 33/53; A61K 39/395; A61K 38/22; A01K 67/027

(52) U.S. Cl. 435/6; 435/7.1; 514/12; 514/44; 424/145.1; 800/14

(57) ABSTRACT

The present invention provides a method of determining a modulator of Y receptor associated bone remodeling and/or bone growth and/or adiposity in a human or animal subject. The present invention further provides a method of determining a modulator of Y receptor associated differentiation of a mesenchymal stem cell into an osteoblast or an adipocyte. The present invention further provides method for the treatment of bone disorders, and in particular osteoporosis, and obesity in addition to compositions for the treatment of said disorders.

### FIELD OF THE INVENTION

The present invention relates to methods of determining a compound that is capable of modulating bone remodeling and/or bone growth. In particular, the present invention provides a method of determining a modulator of Y receptor associated bone remodeling and/or bone growth and/or adiposity in a human or animal subject. Additionally, the present invention provides a method of determining a modulator of Y receptor associated differentiation of a mesenchymal stem cell into an osteoblast or an adipocyte. The present invention also provides methods of treatment of bone disorders, and in particular osteoporosis, and obesity in addition to compositions for the treatment of said disorders.

### BACKGROUND OF THE INVENTION

1. General

This specification contains nucleotide and amino acid sequence information prepared using PatentIn Version 3.1, presented herein after the claims. Each nucleotide sequence is identified in the sequence listing by the numeric indicator <210> followed by the sequence identifier (e.g. <210>1, <210>2, <210>3, etc). The length and type of sequence (DNA, protein (PRT), etc), and source organism for each nucleotide sequence, are indicated by information provided in the numeric indicator fields <211>, <212> and <213>, respectively. Nucleotide sequences referred to in the specification are defined by the term “SEQ ID NO:”, followed by the sequence identifier (eg. SEQ ID NO: 1 refers to the sequence in the sequence listing designated as <400>1).

The designation of nucleotide residues referred to herein are those recommended by the IUPAC-IUB Biochemical Nomenclature Commission, wherein A represents Adenine, C represents Cytosine, G represents Guanine, T represents thymine, Y represents a pyrimidine residue, R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

As used herein the term “derived from” shall be taken to indicate that a specified integer may be obtained from a particular source albeit not necessarily directly from that source.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Unless specifically stated otherwise, each feature described herein with regard to a specific embodiment of the invention, shall be taken to apply mutatis mutandis to each and every other embodiment of the invention.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purposes of exemplification only. Functionally-equivalent products, compositions and methods are clearly within the scope of the invention, as described herein.

The present invention is performed without undue experimentation using, unless otherwise indicated, conventional techniques of molecular biology, microbiology, virology, recombinant DNA technology, peptide synthesis in solution, solid phase peptide synthesis, histology and immunology. Such procedures are described, for example, in the following texts that are incorporated by reference:

Sambrook, Fritsch & Maniatis, Molecular Cloning: A Laboratory Manual, Cold Spring Harbor Laboratories, New York, Second Edition (1989), whole of Vols I, II, and III; DNA Cloning: A Practical Approach, Vols. I and II (D. N. Glover, ed., 1985), IRL Press, Oxford, whole of text;

Oligonucleotide Synthesis: A Practical Approach (M. J. Gait, ed., 1984) IRL Press, Oxford, whole of text, and particularly the papers therein by Gait, ppl-22; Atkinson et al., pp35-81; Sproat et al., pp 83-115; and Wu et al., pp 135-151;

Nucleic Acid Hybridization: A Practical Approach (B. D. Hames & S. J. Higgins, eds., 1985) IRL Press, Oxford, whole of text;

Immobilized Cells and Enzymes: A Practical Approach (1986) IRL Press, Oxford, whole of text;

Perbal, B., A Practical Guide to Molecular Cloning (1984);

Methods In Enzymology (S. Colowick and N. Kaplan, eds., Academic Press, Inc.), whole of series;

J. F. Ramalho Ortigão, “The Chemistry of Peptide Synthesis” In: Knowledge database of Access to Virtual Laboratory website (Interactiva, Germany);

Sakakibara, D., Teichman, J., Lien, E. Land Fenichel, R. L. (1976). Biochem. Biophys. Res. Commun. 73 336-342

Merrifield, R. B. (1963). J. Am. Chem. Soc. 85, 2149-2154.

Barany, G. and Merrifield, R. B. (1979) in The Peptides (Gross, E. and Meienhofer, J. eds.), vol. 2, pp. 1-284, Academic Press, New York.

Wünsch, E., ed. (1974) Synthese von Peptiden in Houben-Weyls Metoden der Organischen Chemie (Müler, E., ed.), vol. 15, 4th edn., Parts 1 and 2, Thieme, Stuttgart.

Bodanszky, M. (1984) Principles of Peptide Synthesis, Springer-Verlag, Heidelberg.

Bodanszky, M. & Bodanszky, A. (1984) The Practice of Peptide Synthesis, Springer-Verlag, Heidelberg.

Bodanszky, M. (1985) Int. J. Peptide Protein Res. 25, 449-474.

Handbook of Experimental Immunology, Vols. I-IV (D. M. Weir and C. C. Blackwell, eds., 1986, Blackwell Scientific Publications).

2. Description of the Related Art

Living bone tissue is continuously being replenished by the processes of resorption and deposition of bone matrix and minerals. This process, termed bone remodeling, is largely accomplished by two cell populations, osteoclasts and osteoblasts. Osteoclasts are recruited from bone marrow or circulation to the bone surface where they break down pre-existing bone matrix and minerals (ie. bone resorption). The bone matrix and minerals are subsequently replaced by osteoblasts that are recruited from bone marrow to the site of bone resorption.

Changes in the numbers of osteoclasts or osteoblasts or the activity of either of these cell types has been associated with a variety of diseases, such as, for example, osteomalacia, hyperostosis and osteoporosis, including involutional osteoporosis, post-menopausal osteoporosis, senile osteoporosis and steroid (glucocorticoid osteoporosis). Each of these diseases is characterized by low bone mass and structural deterioration of the bone tissue resulting in bone fragility and an increased risk of fracture, especially of the hip, spine and wrist.

The most common of the bone diseases, osteoporosis, is a systemic skeletal disease, characterized by low bone mass and deterioration of bone tissue, with a consequent increase in bone fragility and susceptibility to fracture. It is the most common type of metabolic bone disease in the U.S., and the condition has been estimated to affect approximately 10 million people in the United States alone. In addition to those suffering from diagnosed osteoporosis, it is estimated that up to 3 to 4 times this number may have low bone mass placing them at an increased risk of bone fracture.

Osteoporosis causes more than 1.5 million fractures each year, including 700,000 vertebral fractures, 300,000 hip and 250,000 wrist fractures annually, the treatment of which is estimated to cost approximately US$ 17 billion per annum.

In addition to the well-known bone diseases a variety of other conditions are characterized by a need to enhance bone formation. For example, it would be desirable to enhance bone formation and repair in order to treat a common bone fracture. Augmentation of bone formation and/or repair would also be of particular use in the treatment of bone segmental defects, periodontal defects, metastatic bone disease, and osteolytic bone disease (such as, for example, myeloma).

Methods for treating bone disease have varied considerably but to date no satisfactory treatment has been determined.

A conventional treatment of bone disease is the administration of a calcium supplement to a subject to induce bone development. However, to date there is no evidence that calcium has been successful in the treatment of bone disease, in particular treatment using calcium alone has been shown to have little or no effect on inducing bone formation.

Alternative treatments include, for example, a combination of large dosages of vitamin D in combination with fluoride. While this method may result in the rapid production of new bone, in vivo administration of fluoride induces the formation of structurally unsound bone, called woven bone. As a result, patients treated with fluoride suffer from an increased incidence of fractures, in addition to a gastrointestinal reaction to the high dosages of fluoride administered.

U.S. Pat. No. 4,225,496 suggests that various metabolites of vitamin D₃ may be of particular use in increasing calcium resorption and retention within the body of a subject suffering from a physiological tendency to loss of bone mass. While the metabolites described were capable of increasing calcium resorption and retention when used to treat patients they also induced hypercalcinemia, particularly when used in conjunction with a conventional calcium supplement treatment.

Another regularly used treatment for inducing bone accrual is the administration of sex hormones, and in particular estrogen. Estrogen is known to reduce fractures, and is an example of an anti-resorptive agent. In addition, Black, et al. (EP 0605193A1) report that estrogen, particularly when taken orally, lowers plasma levels of low density lipoproteins (LDL's) and raises beneficial high density lipoproteins (HDL's). However, estrogen fails to restore bone to young adult levels in the established osteoporotic skeleton. Moreover, long-term estrogen therapy in patients has been implicated in a variety of disorders, including an increase in the risk of uterine cancer, endometrial cancer and possibly breast cancer.

A further therapy currently used in the treatment of subjects suffering from osteoporosis is the administration of selective estrogen receptor modulators (SERMS), such as, for example, Raloxifene. However, as with the administration of estrogen, the effects of these modulators are not limited to bone, rather these drugs affect many organs and systems of the body. Initial studies with SERMs indicate that they may be effective in the treatment of osteoporosis with an observed increase in bone mineralization and bone density. However, without further study, in an adequate model, the side effects of SERMs (such as, for example, breast cancer and deep vein thrombosis) cannot be assessed.

Whilst there has been significant progress in identification and characterization of putative therapeutics for the treatment of bone diseases, and in particular osteoporosis, there still exists a need for new therapeutics for the treatment of these diseases. Moreover, there exists a need for a model of the human condition that allows researchers to assess the efficacy of new compounds not only on bone remodeling, but also other processes of the body.

Currently, therapeutics are merely screened by determining the effect of the putative therapeutic compound on bone formation in an animal, assays that are performed over extended periods of time (ie. 1 to 8 months depending on the choice of model organism). Alternatively compounds are tested in cell culture, to determine the effect of compounds of bone cell proliferation, an assay that does not consider the effect of the compound on other processes in the body. Accordingly, a more rapid assay for the identification of compounds capable of modulating bone formation would greatly expedite the identification of new lead compounds. In particular, an in vivo assay that facilitates not only determining the effects of a compound on bone formation but also other systems and organs in a subject is particularly desirable.

### SUMMARY OF THE INVENTION

In work leading up to the present invention the inventors developed several murine models that specifically showed consistent large increases in bone formation and in cancellous bone volume. In particular, the present inventors showed that modulation of the level of expression or function of a Y1 receptor, a Y2 receptor, a combination of a Y1 receptor and a Y2 receptor, a combination of a Y1 receptor and a Y4 receptor, a combination of a Y2 receptor and a Y4 receptor or a combination of a Y1 receptor, a Y2 receptor and a Y4 receptor modulated cancellous bone volume, trabecular thickness and trabecular number. The inventors have shown that modulation of the level of expression or the activity of a Y receptor or a combination of Y receptors modulates the activity of osteoblasts resulting in the production of more bone matrix and an increased mineral apposition rate (ie. an increase in bone remodeling).

Surprisingly, the inventors have also found that modulation of Y4 receptor activity dramatically altered the sensitivity of an animal subject to a modulator of bone remodeling.

A further consequence of the reduced expression or activity of a Y receptor in a subject, as exemplified by the phenotypes of mouse models having one or more insertionally inactivated Y receptor genes, includes reduced adiposity.

Surprisingly, the present inventors have also determined that the effect of modulating Y2 receptor activity and/or expression produces a greater effect on bone remodeling, bone growth and adiposity in male subjects compared to female subjects (ie. there is a greater enhancement of bone remodeling or bone growth and a greater reduction in adiposity in male subjects compared to female subjects). As a consequence, one embodiment of the present invention is particularly directed to modulating bone remodeling, bone growth and adiposity in male subjects, such as, for example in the treatment of a bone disease/disorder.

Accordingly, one aspect of the present invention provides methods for determining a modulator of neuropeptide Y receptor associated bone remodeling, bone formation and/or adiposity. In general, the methods of the present invention comprise determining the level of neuropeptide Y receptor expression and/or activity and determining bone remodeling or bone growth or adiposity, wherein one or more changes in one or more of these parameters indicates that the candidate compound is a modulator of Y receptor associated bone remodeling, bone growth or adiposity. As will be apparent to the skilled artisan, known Y receptor modulators that also modulate bone remodeling, bone growth or adiposity would also fall within the scope of a “modulator of Y receptor associated bone remodeling, bone growth or adiposity”. Similarly, any known modulator of bone remodeling, bone growth or adiposity that is shown by the screening assay of the invention to modulate a Y receptor expression or activity would also fall within the scope of a “modulator of Y receptor associated bone remodeling, bone growth or adiposity”. Such compounds clearly constitute a selection that provides advantages in terms of specificity over the mere provision of modulators of Y receptor activity or expression or bone remodeling or bone growth or adiposity.

In one embodiment, the present invention provides a method for determining a modulator of neuropeptide Y receptor associated bone remodeling comprising:
(i) determining the level of neuropeptide Y receptor associated bone remodeling in the presence of a candidate compound; and
(ii) determining the level of neuropeptide Y receptor associated bone remodeling in the absence of a candidate compound,
   wherein a difference in the level of said neuropeptide Y receptor associated bone remodeling at (i) and (ii) indicates that the candidate compound is a modulator of Y receptor associated bone remodeling.

In another embodiment, the present invention provides a method comprising:
(i) determining the level of neuropeptide Y receptor activity and/or expression and the level of bone remodeling in the presence of a candidate compound; and
(ii) determining the level of neuropeptide Y receptor activity and/or expression and the level of bone remodeling in the absence of a candidate compound,
   wherein a difference in the level of said neuropeptide Y receptor activity and/or expression and a difference in the level of bone remodeling at (i) and (ii) indicates that the candidate compound is a modulator of Y receptor associated bone remodeling.

Preferably, cells are isolated from an animal subject and then Y receptor activity and/or expression is determined in the presence and absence of the compound using the isolated cells. Alternatively, the compound is administered to an animal subject and cells are isolated from an animal subject and then Y receptor activity and/or expression is determined in the isolated cells.

Art recognized methods are used to determine Y receptor activity.

In one embodiment, Y receptor activity is determined by performing a process comprising contacting a receptor ligand with the cells in the presence and absence of the compound under conditions sufficient for the ligand to bind to a Y receptor expressed in said cells and determining the binding of the ligand to the Y receptor wherein a difference in binding in the presence and absence of the compound indicates that the compound is a modulator of Y receptor activity. Preferably, the ligand is labeled with a detectable marker.

Preferred ligands include a ligand selected from the group consisting of neuropeptide Y (NPY), pancreatic polypeptide and peptide YY (PYY).

In another embodiment, Y receptor activity is determined by performing a process comprising exposing the cells to an amount of forskolin sufficient in the presence and absence of the compound to induce cAMP accumulation in the cells and determining the amount of cAMP in the cells wherein a difference in cAMP in the presence and absence of the compound indicates that the compound is a modulator of Y receptor activity.

In a still further embodiment, Y receptor activity is determined by performing a process comprising determining calcium mobilization in the cells wherein a difference in calcium mobilization in the presence and absence of the compound indicates that the compound is a modulator of Y receptor activity.

Preferably, calcium mobilization is determined by contacting the cells with a cell-permeable marker that binds intracellular free Ca²⁺ under conditions sufficient for the marker to permeate the cells and then determining the intracellular amount of Ca2⁺ bound to the marker. Preferably, the cell-permeable marker is a fluorescently-labeled marker.

In one embodiment, the method of the present invention comprises isolating bone tissue from an animal subject and then determining bone remodeling in the presence and absence of the compound using the isolated bone tissue or an organ culture derived there from. Preferably, the bone tissue is cultured in an organ culture.

In an alternative embodiment, the method of the present invention comprises administering the compound to an animal subject, isolating bone tissue from an animal subject and then determining bone remodeling in the isolated bone tissue.

Preferably, the bone tissue comprises calvarial (skullcap) bone tissue or femur bone tissue.

In one embodiment, bone remodeling is determined by measuring a parameter in the bone tissue or organ culture derived there from wherein a difference in the measurement of the parameter in the presence and absence of the compound indicates that the compound is a modulator of bone remodeling, and wherein the parameter is selected from the group consisting of bone thickness, amount of new bone, rate of formation of new bone, osteoblast number, osteoclast number, cell proliferation, degree of apoptosis, cortical area, cortical thickness, mineralized bone content, trabecular bone volume, trabecular thickness, trabeculae number, and mineral apposition rate.

In a particularly preferred embodiment, the parameter that is measured in the bone tissue or organ culture derived there from is selected from the group consisting of rate of formation of new bone, osteoblast number, osteoclast number, cortical area, cortical thickness, trabecular bone volume, trabecular thickness, trabeculae number, and mineral apposition rate.

In one embodiment, a method for determining a modulator of neuropeptide Y receptor associated bone remodeling comprises administering a modulator of bone remodeling to an animal subject and determining a change in Y receptor activity, wherein a modified Y receptor activity in the presence of the compound compared to the Y receptor activity in the absence of the compound indicates that the compound is a modulator of Y receptor associated bone remodeling.

In another embodiment, a method for determining a modulator of neuropeptide Y receptor associated bone remodeling comprising administering a modulator of Y receptor activity and/or expression to an animal subject and determining a change in bone remodeling activity, wherein a modified bone remodeling activity in the presence of the compound compared to the bone remodeling activity in the absence of the compound indicates that the compound is a modulator of Y receptor associated bone remodeling.

Preferably, a method of determining neuropeptide Y associated bone remodeling activity is performed in or with an animal subject has a level of bone remodeling activity that is about the same as an animal subject that has not been treated with a compound that modulates bone remodeling or has not been modified at the genetic level to reduce expression of a Y receptor-encoding gene other than a Y4 receptor.

Even more preferably, a method of determining neuropeptide Y bone remodeling activity is performed in or with an animal subject that has been modified at the genetic level to reduce expression of a Y receptor-encoding gene thereby enhancing its sensitivity to a modulator of bone remodeling activity. Preferably, the animal subject has been modified at the genetic level to reduce expression of a Y4 receptor-encoding gene in at least one tissue thereof.

In one embodiment, a method of determining a modulator of Y receptor associated bone remodeling is performed in an aged animal subject.

In another embodiment, The method of determining a modulator of Y receptor associated bone remodeling is performed in an animal subject that suffers from a bone disease characterized by aberrant bone remodeling activity. Preferably, the animal subject suffers from a bone disease selected from the group consisting of osteomalacia, hyperostosis and osteoporosis.

In one embodiment, the animal subject is a gonadectomized animal subject.

In a preferred embodiment, a method of determining neuropeptide Y bone remodeling activity is performed in a male subject.

Preferably, a method of determining neuropeptide Y bone remodeling activity is performed in an animal subject is selected from the group consisting of rat, mouse, chimpanzee, chicken, guinea pig, rabbit, bovine, sheep, and zebrafish. It is particularly preferred that the method is performed in a mouse.

In one embodiment, the Y receptor associated bone remodeling is associated with a Y receptor that is expressed external to the parenchyma of the central nervous system of an animal. Preferably, the Y receptor is at least expressed in the arcuate nucleus of an animal.

In a preferred embodiment the bone remodeling is associated with the activity and/or expression of one or more Y receptors selected from the group consisting of:

(i) a Y1 receptor;

(ii) a Y5 receptor;

(iii) a Y7 receptor;

(iv) a Y1 receptor and a Y2 receptor;

(v) a Y1 receptor and a Y5 receptor;

(vi) a Y1 receptor and a Y7 receptor;

(vii) a Y2 receptor and a Y5 receptor;

(viii) a Y2 receptor and a Y7 receptor;

(ix) a Y5 receptor and a Y7 receptor;

(x) a Y1 receptor and a Y2 receptor and a Y5 receptor;

(xi) a Y1 receptor and a Y2 receptor and a Y7 receptor;

(xii) a Y1 receptor and a Y5 receptor and a Y7 receptor; and

(xiii) a Y1 receptor and a Y2 receptor and a Y5 receptor and a Y7 receptor.

Preferably, the bone remodeling is at least associated with the activity and/or expression of a Y1 receptor.

In a particularly preferred embodiment, the bone remodeling is associated with the activity and/or expression of one or more Y receptors selected from the group consisting of:

(i) a Y1 receptor; and

(ii) a Y1 receptor and a Y2 receptor.

In one embodiment, a method of determining a compound that modulates Y receptor associated bone remodeling comprises the additional step of determining the ability of the compound to pass across the blood brain barrier of an animal subject and selecting a compound that does not efficiently pass the blood brain barrier.

In a preferred embodiment, a compound that modulates neuropeptide Y receptor associated bone remodeling is a small molecule, nucleic acid, protein or antibody that antagonizes the activity and/or expression of a Y receptor. Preferably, the nucleic acid comprises siRNA, PNA, RNAI, ribozyme or antisense RNA.

In another embodiment, the present invention provides a method for determining a modulator of neuropeptide Y receptor associated bone growth comprising:

(i) determining the level of neuropeptide Y receptor associated bone growth in the presence of a candidate compound; and

(ii) determining the level of neuropeptide Y receptor associated bone growth in the absence of a candidate compound,

wherein a difference in the level of said neuropeptide Y receptor associated bone growth at (i) and (ii) indicates that the candidate compound is a modulator of Y receptor associated bone growth.

In another embodiment, the present invention provides a method comprising:
(i) determining the level of neuropeptide Y receptor activity and/or expression and the level of bone growth in the presence of a candidate compound; and
(ii) determining the level of neuropeptide Y receptor activity and/or expression and the level of bone growth in the absence of a candidate compound,
   wherein a difference in the level of said neuropeptide Y receptor activity and/or expression and a difference in the level of bone growth at (i) and (ii) indicates that the candidate compound is a modulator of Y receptor associated bone growth.

Preferably, cells are isolated from an animal subject and then Y receptor activity and/or expression is determined in the presence and absence of the compound using the isolated cells.

Preferably, the animal subject is a juvenile or immature subject or a subject that has not attained sexual maturity or in which the bone remodeling phase has not been attained.

Preferably, a compound is administered to an animal subject, then cells are isolated from an animal subject and then Y receptor activity and/or expression in the isolated cells is determined.

In one embodiment, a method of determining a compound that modulates Y receptor associated bone growth comprises the additional step of isolating bone tissue from an animal subject and then determining bone growth in the presence and absence of the compound using the isolated bone tissue or an organ culture derived there from.

Preferably, the bone tissue is cultured to produce an organ culture.

In another embodiment, a method of determining a compound that modulates Y receptor associated bone growth comprises the additional step of administering the compound to an animal subject, isolating bone tissue from an animal subject and then determining bone growth in the isolated bone tissue. Preferably, the bone tissue comprises calvarial (skullcap) bone tissue or femur bone tissue.

In a preferred embodiment, bone growth is determined by measuring a parameter in the bone tissue or organ culture derived there from wherein a difference in the measurement of the parameter in the presence and absence of the compound indicates that the compound is a modulator of bone length, bone growth, and wherein the parameter is selected from the group consisting of bone thickness, amount of new bone, rate of formation of new bone, osteoblast number, osteoclast number, cell proliferation, degree of apoptosis, cortical area, cortical thickness, mineralized bone content, cancellous bone volume, trabecular bone volume, trabecular thickness, trabeculae number, and mineral apposition rate.

In a particularly preferred embodiment, the bone growth is determined by measuring a parameter selected from the group consisting of trabecular bone volume, trabeculae number, mineral apposition rate, cancellous bone volume, and bone length.

In one embodiment, a method for determining a modulator of neuropeptide Y receptor associated bone growth comprises administering a modulator of bone growth to an animal subject and determining a change in Y receptor activity, wherein a modified Y receptor activity in the presence of the compound compared to the Y receptor activity in the absence of the compound indicates that the compound is a modulator of Y receptor associated bone growth.

In another embodiment, a method for determining a modulator of neuropeptide Y receptor associated bone growth comprising administering a modulator of Y receptor activity and/or expression to an animal subject and determining a change in bone growth activity, wherein a modified bone growth activity in the presence of the compound compared to the bone growth activity in the absence of the compound indicates that the compound is a modulator of Y receptor associated bone growth.

Preferably, a method of determining a compound that modulates Y receptor associated bone growth is performed in an animal subject that has a level of bone growth activity that is about the same as an animal subject that has not been treated with a compound that modulates bone growth or has not been modified at the genetic level to reduce expression of a Y receptor-encoding gene other than a Y4 receptor.

Preferably, a method of determining a compound that modulates Y receptor associated bone growth is performed in an animal subject that has been modified at the genetic level to reduce expression of a Y receptor-encoding gene thereby enhancing its sensitivity to a modulator of bone growth activity. Even more preferably, the animal subject has been modified at the genetic level to reduce expression of a Y4 receptor-encoding gene in at least one tissue thereof. The use of male and/or females subjects is encompassed by the invention.

In another embodiment, a method of determining a compound that modulates Y receptor associated bone growth comprises the step of administering a compound to a pregnant female animal subject for a time and under conditions for the compound to modulate Y receptor associated bone growth in a developing embryo and isolating cells from the developing embryo.

Preferably, an animal subject is selected from the group consisting of rat, mouse, chimpanzee, chicken, guinea pig, rabbit, bovine, sheep, and zebrafish.

In a preferred embodiment, the Y receptor associated bone growth is associated with a Y receptor that is expressed external to the parenchyma of the central nervous system of an animal. Preferably, the Y receptor is at least expressed in the arcuate nucleus of an animal.

In a further preferred embodiment, the bone growth is associated with the activity and/or expression of one or more Y receptors selected from the group consisting of:

(i) a Y1 receptor;

(ii) a Y5 receptor;

(iii) a Y7 receptor;

(iv) a Y1 receptor and a Y2 receptor;

(v) a Y1 receptor and a Y5 receptor;

(vi) a Y1 receptor and a Y7 receptor;

(vii) a Y2 receptor and a Y5 receptor;

(viii) a Y2 receptor and a Y7 receptor;

(ix) a Y5 receptor and a Y7 receptor;

(x) a Y1 receptor and a Y2 receptor and a Y5 receptor;

(xi) a Y1 receptor and a Y2 receptor and a Y7 receptor;

(xii) a Y1 receptor and a Y5 receptor and a Y7 receptor; and

(xiii) a Y1 receptor and a Y2 receptor and a Y5 receptor and a Y7 receptor.

Preferably, the bone growth is at least associated with the activity and/or expression of a Y1 receptor.

In a particularly preferred embodiment, the bone growth is associated with the activity and/or expression of one or more Y receptors selected from the group consisting of:

(i) a Y1 receptor; and

(ii) a Y1 receptor and a Y2 receptor.

In one embodiment, the method of determining a compound that modulates Y receptor mediated bone growth comprises an additional step of determining the ability of the compound to pass across the blood brain barrier of an animal subject and selecting a compound that does not efficiently pass the blood brain barrier.

In a preferred embodiment, the compound is a small molecule, nucleic acid, protein or antibody that antagonizes the activity and/or expression of a Y receptor. Preferably, the nucleic acid comprises siRNA, PNA, RNAI, ribozyme or antisense RNA.

A further embodiment of the present invention provides a method for determining a modulator of neuropeptide Y receptor associated adiposity comprising:
(i) determining the level of neuropeptide Y receptor associated adiposity in the presence of a candidate compound; and
(ii) determining the level of neuropeptide Y receptor associated adiposity in the absence of a candidate compound,
   wherein a difference in the level of said neuropeptide Y receptor associated adiposity at (i) and (ii) indicates that the candidate compound is a modulator of Y receptor associated adiposity.

In one embodiment, the present invention provides a method comprising:
(i) determining the level of neuropeptide Y receptor activity and/or expression and the level of adiposity in the presence of a candidate compound; and
(ii) determining the level of neuropeptide Y receptor activity and/or expression and the level of adiposity in the absence of a candidate compound,
   wherein a difference in the level of said neuropeptide Y receptor activity and/or expression and a difference in the level of adiposity at (i) and (ii) indicates that the candidate compound is a modulator of Y receptor associated adiposity.

Preferably, cells are isolated from an animal subject and Y receptor activity and/or expression is determined in the presence and absence of the compound using the isolated cells.

Even more preferably, the compound is administered to an animal subject, cells are isolated from an animal subject and then Y receptor activity and/or expression is determined in the isolated cells.

Preferably, the method of determining a compound that modulates Y receptor associated adiposity comprises determining the amount of adipose tissue in an animal subject. Preferably, the adipose tissue is white adipose tissue. Even more preferably, the ratio of white adipose tissue to brown adipose tissue is determined.

In one embodiment, a method for determining a modulator of neuropeptide Y receptor associated adiposity comprises administering a modulator of adiposity to an animal subject and determining a change in Y receptor activity, wherein a modified Y receptor activity in the presence of the compound compared to the Y receptor activity in the absence of the compound indicates that the compound is a modulator of Y receptor associated adiposity.

In another embodiment, a method for determining a modulator of neuropeptide Y receptor associated adiposity comprises administering a modulator of Y receptor activity and/or expression to an animal subject and determining a change in adiposity, wherein a modified adiposity in the presence of the compound compared to the adiposity in the absence of the compound indicates that the compound is a modulator of Y receptor associated adiposity.

In a preferred embodiment, the animal subject has a level of adiposity that is about the same as an animal subject that has not been treated with a compound that modulates adiposity or has not been modified at the genetic level to reduce expression of a Y receptor-encoding gene other than a Y4 receptor-encoding gene.

In another embodiment, the animal subject is a gonadectomized animal subject.

In a still further embodiment the animal subject is a male subject.

Preferably, the animal subject is selected from the group consisting of rat, mouse, chimpanzee, chicken, guinea pig, rabbit, bovine, sheep, and zebrafish.

In one embodiment, the Y receptor associated adiposity is associated with a Y receptor that is expressed external to the parenchyma of the central nervous system of an animal. Preferably, the Y receptor is at least expressed in the arcuate nucleus of an animal.

In a preferred embodiment, the Y receptor associated adiposity is associated with the activity and/or expression of one or more Y receptors selected from the group consisting of:

(i) a Y7 receptor;

(ii) a Y1 receptor and a Y2 receptor;

(iii) a Y1 receptor and a Y4 receptor;

(iv) a Y1 receptor and a Y5 receptor;

(v) a Y1 receptor and a Y7 receptor;

(vi) a Y2 receptor and a Y4 receptor;

(vii) a Y2 receptor and a Y5 receptor;

(viii) a Y2 receptor and a Y7 receptor;

(ix) a Y4 receptor and a Y5 receptor;

(x) a Y4 receptor and a Y7 receptor;

(xi) a Y5 receptor and a Y7 receptor;

(xii) a Y1 receptor and a Y2 receptor and a Y4 receptor;

(xiii) a Y1 receptor and a Y2 receptor and a Y5 receptor;

(xiii) a Y1 receptor and a Y2 receptor and a Y7 receptor;

(xiv) a Y1 receptor and a Y4 receptor and a Y5 receptor;

(xv) a Y1 receptor and a Y4 receptor and a Y7 receptor;

(xvi) a Y1 receptor and a Y5 receptor and a Y7 receptor;

(xvii) a Y2 receptor and a Y4 receptor and a Y5 receptor;

(xviii) a Y2 receptor and a Y4 receptor and a Y7 receptor;

(xix) a Y2 receptor and a Y5 receptor and a Y7 receptor;

(xx) a Y4 receptor and a Y5 receptor and a Y7 receptor; and

(xxi) a Y1 receptor and a Y4 receptor and a Y5 receptor and a Y7 receptor.

Preferably, the Y receptor associated adiposity is at least associated with the activity and/or expression of a Y7 receptor. Even more preferably, the Y receptor associated adiposity is associated with the activity and/or expression of a Y2 receptor and a Y4 receptor.

In one embodiment, a method of determining a compound that modulates Y receptor associated adiposity comprises an additional step of determining the ability of the compound to pass across the blood brain barrier of an animal subject and selecting a compound that does not efficiently pass the blood brain barrier.

Preferably. the compound is a small molecule, nucleic acid, protein or antibody that antagonizes the activity and/or expression of a Y receptor. Preferably, the nucleic acid comprises siRNA, PNA, RNAI, ribozyme or antisense RNA.

A further embodiment of the present invention provides a method of determining a compound that is a modulator of Y receptor associated differentiation of a mesenchymal stern cell (MSC) or bone marrow stromal cell (BMSC) into an osteoblast-type cell comprising:
(i) culturing a MSC or BMSC in the presence of a candidate compound;
(ii) culturing a MSC or BMSC in the absence of the candidate compound; and
(iii) determining Y receptor activity and/or expression and the number of differentiated osteoblast-type cells at (i) and (ii), wherein a modified number of osteoblast-type cells and a modified Y receptor activity and/or expression at (i) and (ii) indicates that the compound is a modulator of Y receptor associated differentiation of an osteoblast-type cell.

A further embodiment of the present invention provides a method of determining a compound that is a modulator of Y receptor associated differentiation of a mesenchymal stem cell (MSC) or bone marrow stromal cell (BMSC) into an adipocyte-type cell comprising:
(i) culturing a MSC or BMSC in the presence of a candidate compound;
(ii) culturing a MSC or BMSC in the absence of the candidate compound; and
(iii) determining Y receptor activity and/or expression and the number of differentiated adipocyte-type cells at (i) and (ii), wherein a modified number of adipocyte-type cells and a modified Y receptor activity and/or expression at (i) and (ii) indicates that the compound is a modulator of Y receptor associated differentiation of an adipocyte-type cell.

In an alternative embodiment, the ability of the compound to pass across the blood brain barrier of an animal subject is determined and a compound that does not efficiently pass the blood brain barrier is selected.

The present invention clearly encompasses the further step of isolating the MSC or BMSC from an animal subject in the performance of these methods. The MSC or BMSC are isolated from bone marrow of the subject or alternatively, from adipose tissue of the subject.

In one embodiment of these ex vivo methods cells isolated from a human subject or an other animal subject (eg., a gonadectomized animal subject) can be used. As with other embodiments of the invention described herein, the use of a rat, mouse, chimpanzee, chicken, guinea pig, rabbit, bovine, sheep, or zebrafish is preferred. As with other embodiments of the invention described herein, male subjects are preferred.

In one embodiment, the Y receptor associated differentiation is associated with a Y receptor that is expressed external to the parenchyma of the central nervous system of an animal and preferably at least expressed in the arcuate nucleus of an animal.

In one embodiment, differentiation of a MSC or BMSC is associated with the activity and/or expression of one or more Y receptors selected from the group consisting of:

(i) a Y1 receptor;

(ii) a Y5 receptor;

(iii) a Y7 receptor;

(iv) a Y1 receptor and a Y2 receptor;

(v) a Y1 receptor and a Y5 receptor;

(vi) a Y1 receptor and a Y7 receptor;

(vii) a Y2 receptor and a Y5 receptor;

(viii) a Y2 receptor and a Y7 receptor;

(ix) a Y5 receptor and a Y7 receptor;

(x) a Y1 receptor and a Y2 receptor and a Y5 receptor;

(xi) a Y1 receptor and a Y2 receptor and a Y7 receptor;

(xii) a Y1 receptor and a Y5 receptor and a Y7 receptor; and

(xiii) a Y1 receptor and a Y2 receptor and a Y5 receptor and a Y7 receptor.

Preferred compounds obtained by the inventive methods described herein are small molecules, nucleic acids, proteins or antibodies that antagonize the activity and/or expression of a Y receptor. Preferred nucleic acid antagonists comprise siRNA, PNA, RNAi, ribozyme or antisense RNA.

A further embodiment of the present invention provides a non-naturally occurring transformed animal having reduced expression of multiple Y receptors in a cell or tissue by virtue of carrying insertions in multiple Y receptor-encoding genes wherein said animal has modulated bone remodeling activity compared to an otherwise isogeric animal that does not carry the insertions.

A further embodiment of the present invention provides a non-naturally occurring transformed animal having reduced expression of multiple Y receptors in a cell or tissue by virtue of carrying insertions in multiple Y receptor-encoding genes wherein said animal has modulated bone growth activity compared to an otherwise isogenic animal that does not carry the insertions.

A further embodiment of the present invention provides a non-naturally occurring transformed animal having reduced expression of multiple Y receptors in a cell or tissue by virtue of carrying insertions in multiple Y receptor-encoding genes wherein said animal has modulated adiposity compared to an otherwise isogenic animal that does not carry the insertions.

Preferably, the non-naturally occurring transformed animal supra carries insertions in multiple Y receptor encoding genes selected from the group consisting of:

(i) a Y1 receptor and a Y2 receptor;

(ii) a Y1 receptor and a Y5 receptor;

(iii) a Y1 receptor and a Y7 receptor;

(iv) a Y2 receptor and a Y5 receptor;

(v) a Y2 receptor and a Y7 receptor;

(vi) a Y5 receptor and a Y7 receptor;

(vii) a Y1 receptor and a Y2 receptor and a Y5 receptor;

(viii) a Y1 receptor and a Y2 receptor and a Y7 receptor;

(ix) a Y1 receptor and a Y5 receptor and a Y7 receptor; and

(x) a Y1 receptor and a Y2 receptor and a Y5 receptor and a Y7 receptor.

Still more preferably, the non-naturally occurring transformed animal supra carries insertions in multiple Y receptor encoding genes selected from the group consisting of:

(i) a Y1 receptor and a Y2 receptor;

(ii) a Y2 receptor and a Y4 receptor; and

(iii) a Y1 receptor and a Y2 receptor and a Y4 receptor.

Preferably, the non-naturally occurring transformed animal is a mouse. Other animal models are not to be excluded.

The present invention clearly extends to any progeny of the non-naturally occurring transformed animal supra wherein said progeny animal has reduced expression of multiple Y receptors in a cell or tissue by virtue of carrying insertions in multiple Y receptor-encoding genes.

A further embodiment of the present invention extends to the use of the non-naturally occurring transformed animal supra or a progeny animal thereof to determine bone remodeling activity, bone growth or adiposity or an effect of a compound on bone remodeling activity, bone growth or adiposity.

A further embodiment of the present invention provides for the use of a non-naturally occurring transformed animal or progeny thereof having reduced expression of a Y4 receptor in a cell or tissue by virtue of carrying an insertion in the Y4 receptor-encoding gene to determine bone remodeling activity, bone growth or adiposity or an effect of a compound on bone remodeling activity, bone growth or adiposity.

A still further embodiment of the present invention provides a method of treatment of a disorder associated with bone remodeling and/or bone formation comprising administering to a subject in need of treatment an amount of a compound sufficient to modulate Y receptor associated bone remodeling in a cell of the subject. Preferably, the compound antagonizes Y receptor associated bone remodeling.

A still further embodiment of the present invention provides a method of treatment of aberrant adiposity in a subject in need of treatment comprising administering to the subject an amount of a compound that modulates Y receptor associated adiposity sufficient to modulate Y receptor adiposity in a cell of the subject.

A still further embodiment of the present invention provides a method of treatment of aberrant bone remodeling in a subject in need thereof comprising isolating a mesenchymal stem cell (MSC) or bone marrow stromal cell (BMSC) from a human or animal subject, treating the MSC or BMSC with a compound that modulates Y receptor associated differentiation under conditions sufficient to induce differentiation of the MSC or BMSC into an osteoblast type cell and introducing the osteoblast type cell into the subject in need of treatment.

In one embodiment, the aberrant bone remodeling is associated with a bone disorder selected from the group consisting of osteomalacia, hyperostosis, osteoporosis, a bone segmental defect, periodontal defect, metastatic bone disease, and osteolytic bone disease. In accordance with this embodiment, the compound enhances Y receptor associated MSC or BMSC differentiation into an osteoblast type cell.

In an alternative embodiment, the aberrant bone remodeling is associated with osteopetrosis. In accordance with this embodiment, the compound antagonizes Y receptor associated MSC or BMSC differentiation into an osteoblast type cell.

In one embodiment, treatment further comprises expanding or growing the BMSC cells, MSC cells or osteoblast type cells.

In another embodiment, treatment comprises introducing differentiated osteoblast type cells directly into the bone of the subject, eg., by surgical means or by infusing the cells into the blood stream of the subject under conditions sufficient for said osteoblast type cells to be recruited to a bone of the subject.

A still further embodiment of the present invention provides for the use of an antagonist of Y receptor associated bone remodeling in the preparation of a medicament for the treatment of aberrant bone remodeling in an animal or human subject.

### BRIEF DESCRIPTION OF THE DRAWINGS

FIG. 1: Effect of the Y2 receptor on bone remodeling.

(A)-(C) Reduced expression of the Y2 receptor induced increased bone formation. Sagittal micrographs of the distal femoral metaphysis of wild-type (wt) (A), Y2^{-/+} (B) and Y2^{-/-} (C) mice at 15-17 weeks of age. Sections of bone taken from Y2^{-/-} mice show an increased amount of darkly stained bone compared to bones taken from Y2^{-/+} and wt mice.

FIG. 2: Effect of Y2 receptor on trabecular bone volume.

Reduced expression of the Y2 receptor increased the thickness of trabecular bone (at the distal femoral metaphysis). The volume of trabecular bone isolated from germline Y2−/− was significantly increased compared to the volume of trabecular bone isolated from wt and Y2^{-/+} mice. Values are mean±standard deviation. *P<0.05.

FIG. 3: Effect of Y2 receptor on trabecular bone.

(A) Reduced expression of the Y2 receptor increased the number of trabeculae at the distal femoral metaphysis. Sections of the distal femoral metaphysis were stained for mineralized bone and the number of trabeculae estimated. Germline Y2^{-/-} mice displayed a dramatic increase in the number of trabeculae compared to bones derived from wild type and Y2^{-/+} mice. Values are mean±standard deviation. *P<0.05.

(B) Reduced expression of the Y2 receptor significantly increased trabecular thickness at the distal femoral metaphysis. Sections of the distal femoral metaphysis were stained for mineralized bone and the average trabecular thickness determined. Germline Y2^{-/-} mice displayed a dramatic increase in trabecular thickness compared to ones derived from wild type and Y2^{-/+} mice. Values are mean±standard deviation. *P<0.05.

FIG. 4: Expression of GFP in a mouse injected with a recombinant adenovirus. Fluorescent micrography showed the expression of GFP in a Y2^{lox/lox} mouse 21 days after injection of an recombinant adenovirus expressing GFP. Note the localized expression to the hypothalamus. Bar represents 1 mm.

FIG. 5: Effect of hypothalamic expression of Y2 on bone remodeling

(A)-(C) Reduced expression of the Y2 receptor in the hypothalamus in a mouse induced increased bone formation. Sagittal micrographs of the distal femoral metaphysis of wt (A), GFP-Y2^{lox/lox} (B) and Cre-Y2^{lox/lox} (C) mice at 15-17 weeks of age. Sections of bone taken from Cre-Y2^{lox/lox} mice show an increased amount of darkly stained bone compared to bones taken from GFP-Y2^{lox/lox} and wt mice. Note GFP-Y2^{lox/lox} mice comprise a floxed Y2 gene, however are injected with an adenovirus expressing GFP. Cre-Y2^{lox/lox} mice comprise a floxed Y2 gene and are injected with a Cre expressing adenovirus thereby sliencing Y2 expression in the hypothalamus.

FIG. 6: Effect of hypothalamic expression of Y2 on bone remodeling

(A) Reduced expression of the Y2 receptor in the hypothalamus in a mouse induced a dramatic increase in trabecular bone volume. The volume of trabecular bone isolated from conditional Y2^{-/-} (as a percentage of total bone volume) was significantly increased compared to the volume of trabecular bone isolated from wt and GFP-Y2^{lox/lox} mice. Values are mean±standard deviation. **P<0.005.

(B) Reduced expression of the Y2 receptor increased the number of trabeculae at the distal femoral metaphysis. Sections of the distal femoral metaphysis were stained for mineralized bone and the number of trabeculae per mm of bone was estimated. Conditional Y2^{-/-} mice displayed a dramatic increase in the number of trabeculae compared to bones derived from wild type and GFP-Y2^{lox/lox} mice. Values are mean s standard deviation. *P<0.05.

(C) Reduced expression of the Y2 receptor significantly increased trabecular thickness at the distal femoral metaphysis. Sections of the distal femoral metaphysis were stained for mineralized bone and the average trabecular thickness determined. Conditional Y2^{-/-} mice displayed a dramatic increase in trabecular thickness compared to bones derived from wild type and GFP-Y2^{lox/lox} mice. Values are mean±standard deviation. *P<0.05.

FIG. 7: Effect of reduced expression of Y2 receptors in the hypothalamus on trabecular bone cell activity.

(A) Sections of the distal femoral metaphysis were stained to detect the osteoclast surface and it was shown that conditional Y2^{-/-} mice showed no change in the size of the osteoclast surface (as a percentage of total bone volume) compared to wt controls. Values are mean±standard deviation.

(B) Sections of the distal femoral metaphysis were stained to detect osteoclast cells. Conditional Y2^{-/-} mice displayed a significantly reduced number of osteoclasts per mm of bone compared to wt controls. Values are mean±standard deviation. *P<0.05.

(C) Sections of the distal femoral metaphysis were stained to detect the osteoblast surface and it was shown that conditional Y2^{-/-} mice showed no change in the size of the osteoclast surface (as a percentage of total bone volume) compared to wt controls. Values are mean=standard deviation.

(D) Sections of the distal femoral metaphysis were stained to detect osteoblast cells. Conditional Y2^{-/-} mice displayed no change in the number of osteoclasts per mm of bone compared to wt controls. Values are mean±standard deviation.

FIG. 8: Reduced expression of Y2 receptors in the hypothalamus increased bone mineralization rate.

(A)-(B) Conditional Y2^{-/-} and wt mice were injected with fluorescent markers to detect the rate of mineral apposition in bone. As shown the distance between the to fluorescent dye markers is increased in conditional Y2^{-/-} (FIG. 8B) compared to wt mice (FIG. 8A), indicating that conditional Y2^{-/-} mice formed more bone in a period of 7 days compared to wt controls.

FIG. 9: Reduced expression of Y2 receptors in the hypothalamus increased bone formation.

(A) (B) Quantification of the rate of mineral apposition (shown in FIG. 8). The change in the distance between fluorescent markers that are used to assess bone formation was determined. Conditional Y2^{-/-} mice form significantly more bone per day than a wt control mouse (A). This result is reflected in the rate at which conditional Y2^{-/-} mice form new bone compared to wt mice (B). Values are mean±standard deviation. *P<0.05.

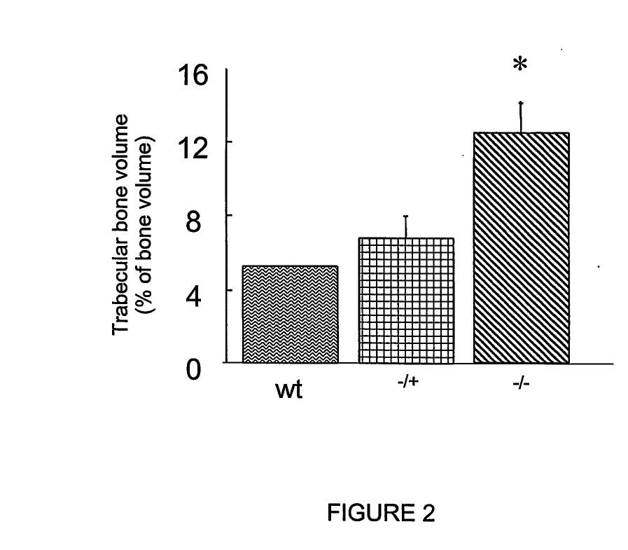

FIG. 10: FIG. 2: Effect of Y4 receptor on trabecular bone volume.

Reduced expression of the Y4 receptor had no effect on trabecular bone volume compared to wt control mice. Accordingly, the volume of Y2^{-/-} trabeculae was significantly larger then Y4^{-/-} mice. Values are mean±standard deviation. *P<0.05.

FIG. 11: Effect of Y2 and Y4 receptors on bone remodeling.

(A)-(D) Sagittal micrographs of the distal femoral metaphysis of wild-type (wt) (A), Y4^{-/-} (B), Y2^{-/-} (C) and Y2^{-/-}Y4^{-/-} (D) mice at 15-17 weeks of age. Sections of bone taken from Y2^{-/-} mice show an increased amount of darkly stained bone compared to bones taken from Y4^{-/-} and wt mice. Sections of bone taken from Y2^{-/-}Y4^{-/-} mice show a further increased in the amount of darkly stained bone compared to Y2^{-/-} mice.

FIG. 12: Effect of Y2 and Y4 receptors on trabecular bone volume.

The volume of trabecular bone isolated from germline Y2^{-/-} was significantly increased compared to the volume of trabecular bone isolated from wt and Y4^{-/-} mice, while the trabecular volume of Y2^{-/-}Y4^{-/-} mice was further increased above that of Y2^{-/-} mice. Values are mean±standard deviation. *P<0.05 versus wt.

FIG. 13: Effect of Y2 receptor on trabecular bone.

(A) Sections of the distal femoral metaphysis were stained for mineralized bone and the number of trabeculae estimated. Germline Y2^{-/-} mice displayed a dramatic increase in the number of trabeculae compared to bones derived from wild type and Y4^{-/-} mice. The number of trabeculae detected in Y2^{-/-}Y4^{-/-} mice was substantially increased compared to wt, Y4^{-/-} and Y2^{-/-} mice. Values are mean±standard deviation. *P<0.05 versus wt.

(B) Sections of the distal femoral metaphysis were stained for mineralized bone and the average trabecular thickness determined. Germline Y2^{-/-} mice displayed a dramatic increase in trabecular thickness compared to bones derived from wild type and Y4^{-/-} mice, while double knockout mice (Y2^{-/-}Y4^{-/-}) mice showed an increase in trabecular thickness above the values determined for Y2^{-/-} mice. Values are mean±standard deviation. *P<0.05 compared to wt.

FIG. 14: Effect of Y2Y4 deficiency on cortical bone.

(A) Y2^{-/-}Y4^{-/-} mice have reduced cortical bone area. Femora were bisected transversely at the midpoint of the shaft and cortical area determined by subtraction of the medullary area from the total area. Y2^{-/-} mice and Y4^{-/-} mice show no change in cortical area compared to wt, while Y2^{-/-}Y4^{-/-} mice show a significant reduction in cortical area. Values are mean±standard deviation. *P<0.05 compared to wt, Y2^{-/-} or Y4^{-/-}.

(B) Y2^{-/-}Y4^{-/-} mice have reduced cortical bone thickness. Femora were bisected transversely at the midpoint of the shaft and cortical thickness determined by radial difference of the medullary area and the total area. Y2^{-/-} mice and Y4^{-/-} mice show no change in cortical thickness compared to wt, while Y2^{-/-}Y4^{-/-} mice show a significant reduction in cortical thickness. Values are mean±standard deviation. *P<0.05 compared to wt, Y2^{-/-} or Y4^{-/-}.

FIG. 15: Y1 receptor increases cancellous bone volume.

Reduced expression of the Y1 receptor increased the volume of cancellous bone (at the distal femoral metaphysis). The volume of cancellous bone isolated from germline Y1^{-/-} mice was significantly increased compared to the volume of cancellous bone isolated from wt and Y4^{-/-} mice. The cancellous bone volume of Y1^{-/-} mice did not vary significantly from the cancellous bone volume of Y2^{-/-} mice. Values are mean±standard deviation. *P<0.05 versus control.

FIG. 16: Effect of suppressing the expression of multiple Y receptors on cancellous bone volume. As shown in FIG. 15 reduced expression of Y1 and Y2 receptors increases cancellous bone volume, in addition, deficiency in a combination of Y1Y2, Y1Y4, Y2Y4 and Y1Y2Y4 receptors increases cancellous bone volume in an animal subject. Values are mean±standard deviation. *P<0.05 versus wt.

FIG. 17: Effect of suppressing the expression of multiple Y receptors on trabecular thickness. Sections of the distal femoral metaphysis were stained for mineralized bone and the number of trabeculae estimated. Mice deficient in expression of Y1 receptor, Y2 receptor, Y1Y2 receptors, Y1Y4 receptors, Y2Y4 receptors and Y1Y2Y4 receptors had significantly increased trabecular thickness compared to wt or Y4^{-/-} mice. Values are mean±standard deviation. *P<0.05 versus wt.

FIG. 18: Effect of suppressing the expression of multiple Y receptors on cortical area and volume.

(A) Femora were bisected transversely at the midpoint of the shaft and cortical area determined by subtraction of the medullary area from the total area. While Y2^{-/-}Y4^{-/-} and Y1^{-/-}Y4^{-/-} mice show no change in cortical bone area, Y2^{-/-}Y4^{-/-} and Y1^{-/-}Y2^{-/-}Y4^{-/-} mice show a significant reduction in cortical area compared to wt controls. Values are mean±standard deviation. *P<0.05 versus wt.

(B) Femora were bisected transversely at the midpoint of the shaft and cortical thickness determined by radial difference of the medullary area and the total area. While Y2^{-/-}Y4^{-/-} and Y1^{-/-}Y4^{-/-} mice show no change in cortical bone thickness, Y2^{-/-}Y4^{-/-} and Y1^{-/-}/Y2^{-/-}Y4^{-/-} mice show a significant reduction in cortical thickness compared to wt controls. Values are mean±standard deviation. *P<0.05 versus wt.

FIG. 19 Sex differences in Y receptor associated bone remodeling.

(A)-(B) Cancellous bone volume was determined in male (A) and female (13) Y2−−/, Y4^{-/-} and Y2^{-/-}Y4^{-/-} mice. While Y2^{-/-} and Y2^{-/-/Y}4^{-/-} mice showed a significant increase in cancellous bone volume, the cancellous bone volume of Y2^{-/-}Y4^{-/-} male mice (A) was increased above that of Y2^{-/-} female mice (13). Values are mean±standard deviation. *P<0.05 versus wt; #p<0.05 versus Y2^{-/-.}

FIG. 20: Sex of the animal subject does not modulate osteoblast surface or osteoclast surface.

(A)-(B) Sections of the distal femoral metaphysis were stained to detect the osteoblast surface of Y2^{-/-}Y4^{-/-} male and female mice. While the osteoblast surface was increased (compared to total bone surface) in Y2^{-/-}Y4^{-/-} mice, there was no change observed between male (A) and female (B) mice. Values are mean±standard deviation. *P<0.05 versus wt.

(C)-(D) Sections of the distal femoral metaphysis were stained to detect the osteoclast surface of Y2^{-/-}Y4^{-/-} male and female mice. While the osteoclast surface was increased (compared to total bone surface) in Y2^{-/-}Y^{-/-} mice, there was no change observed between male (A) and female (B) mice. Values are mean±standard deviation. *P<0.05 versus wt.

FIG. 21: Mineral apposition rate is increased in male Y2^{-/-}Y4^{-/-} mice.

(A)-(B) wt and Y2^{-/-}Y4^{-/-} male and female mice were injected with fluorescent markers to detect the rate of mineral apposition in bone. As shown mineral apposition rate was increased in both male (A) and female (B) Y2^{-/-}Y4^{-/-} mice. The rate of mineral apposition was increased to a larger degree in male Y2^{-/-}Y4^{-/-} mice than female Y2^{-/-}Y4^{-/-} mice. Values are mean±standard deviation. *P<0.05 versus wt.

FIG. 22: Cortical area is reduced in male Y2^{-/-}Y4^{-/-} mice.

(A)-(B) Femora were bisected transversely at the midpoint of the shaft and cortical area determined by subtraction of the medullary area from the total area. Male Y2^{-/-}Y4^{-/-}(A) mice had significantly reduced mean cortical area compared to wt, while female Y2^{-/-}Y4^{-/-} mice showed no significant change (B). Values are mean±standard deviation. *P<0.05 versus wt.

FIG. 23: Bodyweight is reduced in mice deficient for Y2^{-/-} and Y4^{-/-} receptors.

(A)-(B) Bodyweight was significantly reduced in Y2^{-/-}Y4^{-/-} male (A) and female (B) mice compared to wt controls. This reduced bodyweight was caused by reduced adiposity. Male mice showed a larger reduction in bodyweight than female mice.

### DETAILED DESCRIPTION OF THE PREFERRED EMBODIMENTS

Screening Methods

One embodiment of the present invention provides a method for determining a modulator of neuropeptide Y receptor associated bone remodeling, bone formation and/or adiposity comprising:
(iii) determining the level of neuropeptide Y receptor associated bone remodeling, bone formation and/or adiposity in the presence of a candidate compound; and
(iv) determining the level of neuropeptide Y receptor associated bone remodeling, bone formation and/or adiposity in the absence of a candidate compound,
   wherein a difference in the level of said neuropeptide Y receptor associated bone remodeling, bone formation and/or adiposity at (i) and (ii) indicates that the candidate compound is a modulator of Y receptor associated bone remodeling, bone formation and/or adiposity .

As exemplified by the present inventors, modulation of a Y receptor activity or a combination of Y receptor activities modulates bone formation in a developing animal subject, modulates bone remodeling in an adult animal subject, and modulates adiposity in an animal subject.

In one embodiment, a compound inhibits a neuropeptide Y receptor selected from the group consisting of a Y1 receptor, a Y2 receptor, a Y4 receptor, a Y5 receptor, a y6 receptor, a Y7 receptor and a combination of any of these receptors. Preferably, a neuropeptide Y receptor is selected from the group consisting of a Y1 receptor, a Y4 receptor, a Y5 receptor, a y6 receptor, a Y7 receptor and a combination of any of these receptors.

In another embodiment, a compound inhibits a neuropeptide receptor selected from the group consisting of a Y1 receptor, a Y2 receptor, a Y4 receptor, a Y5 receptor, a y6 receptor, a Y7 receptor and a combination of Y receptors selected from the group consisting of a Y1 receptor and a Y2 receptor; a Y1 receptor, a Y2 receptor and a Y4 receptor; a Y1 receptor, a Y2 receptor and a Y5 receptor; a Y1 receptor, a Y2 receptor and a y6 receptor; a Y1 receptor, a Y2 receptor and a Y7 receptor; a Y1 receptor, a Y2 receptor, a Y4 receptor and a Y5 receptor; a Y1 receptor, a Y2 receptor, a Y4 receptor and a y6 receptor; a Y1 receptor, a Y2 receptor, a Y4 receptor and a Y7 receptor; a Y1 receptor, a Y2 receptor, a Y5 receptor and a y6 receptor; a Y1 receptor, a Y2 receptor, a Y5 receptor and a Y7 receptor; a Y1 receptor, a Y2 receptor, a y6 receptor and a Y7 receptor; a Y1 receptor, a Y2 receptor, a Y4 receptor, a Y5 receptor and a y6 receptor; a Y1 receptor, a Y2 receptor, a Y4 receptor, a Y5 receptor and a Y7 receptor; a Y1 receptor, a Y2 receptor, a Y4 receptor, a Y5 receptor, a y6 receptor and a Y7 receptor; a Y1 receptor and a Y4 receptor; a Y1 receptor, a Y4 receptor and a Y5 receptor; a Y1 receptor, a Y4 receptor and a y6 receptor; a Y1 receptor, a Y4 receptor and a Y7 receptor; a Y1 receptor, a Y4 receptor, a Y5 receptor and a y6 receptor; a Y1 receptor, a Y4 receptor, a Y5 receptor and a Y7 receptor; a Y1 receptor, a Y4 receptor, a y6 receptor and a Y7 receptor; a Y1 receptor, a Y4 receptor, a Y5 receptor and a y6 receptor, a Y7 receptor; a Y1 receptor and a Y5 receptor; a Y1 receptor, a Y5 receptor and a y6 receptor; a Y1 receptor, a Y5 receptor and a Y7 receptor; a Y1 receptor, a Y5 receptor, a y6 receptor and a Y7 receptor; a Y1 receptor and a y6 receptor; a Y1 receptor, a y6 receptor and a Y7 receptor; a Y1 receptor and a Y7 receptor; a Y2 receptor and a Y4 receptor; a Y2 receptor and a Y5 receptor; a Y2 receptor and a y6 receptor; a Y2 receptor and a Y7 receptor; a Y2 receptor, a Y4 receptor and a Y5 receptor; a Y2 receptor, a Y4 receptor and a y6 receptor; a Y2 receptor, a Y4 receptor and a Y7 receptor; a Y2 receptor, a Y5 receptor and a y6 receptor; a Y2 receptor, a Y5 receptor and a Y7 receptor; a Y2 receptor, a y6 receptor and a Y7 receptor; a Y2 receptor, a Y4 receptor, a Y5 receptor and a y6 receptor; a Y2 receptor, a Y4 receptor, a Y5 receptor and a Y7 receptor; a Y2 receptor, a Y4 receptor and a y6 receptor, a Y7 receptor; a Y2 receptor, a Y4 receptor, a Y5 receptor, a y6 receptor and a Y7 receptor; a Y4 receptor and a Y5 receptor; a Y4 receptor and a y6 receptor; a Y4 receptor and a Y7 receptor; a Y4 receptor, a Y5 receptor and a y6 receptor; a Y4 receptor, a Y5 receptor and a Y7 receptor; a Y4 receptor, a y6 receptor and a Y7 receptor; a Y4 receptor, a Y5 receptor, a y6 receptor, a Y7 receptor; a Y5 receptor and a y6 receptor; a Y5 receptor and a Y7 receptor; a Y5 receptor, a y6 receptor and a Y7 receptor; and a y6 receptor and a Y7 receptor.

In a still further embodiment, a compound inhibits a neuropeptide receptor selected from the group consisting of a Y1 receptor, a Y4 receptor, a Y5 receptor, a y6 receptor, a Y7 receptor and a combination of Y receptors selected from the group consisting of a Y1 receptor and a Y2 receptor; a Y1 receptor, a Y2 receptor and a Y4 receptor; a Y1 receptor, a Y2 receptor and a Y5 receptor; a Y1 receptor, a Y2 receptor and a y6 receptor; a Y1 receptor, a Y2 receptor and a Y7 receptor; a Y1 receptor, a Y2 receptor, a Y4 receptor and a Y5 receptor; a Y1 receptor, a Y2 receptor, a Y4 receptor and a y6 receptor; a Y1 receptor, a Y2 receptor, a Y4 receptor and a Y7 receptor; a Y1 receptor, a Y2 receptor, a Y5 receptor and a y6 receptor; a Y1 receptor, a Y2 receptor, a Y5 receptor and a Y7 receptor; a Y1 receptor, a Y2 receptor, a y6 receptor and a Y7 receptor; a Y1 receptor, a Y2 receptor, a Y4 receptor, a Y5 receptor and a y6 receptor; a Y1 receptor, a Y2 receptor, a Y4 receptor, a Y5 receptor and a Y7 receptor; a Y1 receptor, a Y2 receptor, a Y4 receptor, a Y5 receptor, a y6 receptor and a Y7 receptor; a Y1 receptor and a Y4 receptor; a Y1 receptor, a Y4 receptor and a Y5 receptor; a Y1 receptor, a Y4 receptor and a y6 receptor; a Y1 receptor, a Y4 receptor and a Y7 receptor; a Y1 receptor, a Y4 receptor, a Y5 receptor and a y6 receptor; a Y1 receptor, a Y4 receptor, a Y5 receptor and a Y7 receptor; a Y1 receptor, a Y4 receptor, a y6 receptor and a Y7 receptor; a Y1 receptor, a Y4 receptor, a Y5 receptor and a y6 receptor, a Y7 receptor; a Y1 receptor and a Y5 receptor; a Y1 receptor, a Y5 receptor and a y6 receptor; a Y1 receptor, a Y5 receptor and a Y7 receptor; a Y1 receptor, a Y5 receptor, a y6 receptor and a Y7 receptor; a Y1 receptor and a y6 receptor; a Y1 receptor, a y6 receptor and a Y7 receptor; a Y1 receptor and a Y7 receptor; a Y2 receptor and a Y4 receptor; a Y2 receptor and a Y5 receptor; a Y2 receptor and a y6 receptor; a Y2 receptor and a Y7 receptor; a Y2 receptor, a Y4 receptor and a Y5 receptor; a Y2 receptor, a Y4 receptor and a y6 receptor; a Y2 receptor, a Y4 receptor and a Y7 receptor; a Y2 receptor, a Y5 receptor and a y6 receptor, a Y2 receptor, a Y5 receptor and a Y7 receptor; a Y2 receptor, a y6 receptor and a Y7 receptor; a Y2 receptor, a Y4 receptor, a Y5 receptor and a y6 receptor; a Y2 receptor, a Y4 receptor, a Y5 receptor and a Y7 receptor; a Y2 receptor, a Y4 receptor and a y6 receptor, a Y7 receptor; a Y2 receptor, a Y4 receptor, a Y5 receptor, a y6 receptor and a Y7 receptor; a Y4 receptor and a Y5 receptor; a Y4 receptor and a y6 receptor; a Y4 receptor and a Y7 receptor; a Y4 receptor, a Y5 receptor and a y6 receptor; a Y4 receptor, a Y5 receptor and a Y7 receptor; a Y4 receptor, a y6 receptor and a Y7 receptor; a Y4 receptor, a Y5 receptor, a y6 receptor, a Y7 receptor; a Y5 receptor and a y6 receptor; a Y5 receptor and a Y7 receptor; a Y5 receptor, a y6 receptor and a Y7 receptor; and a y6 receptor and a Y7 receptor.

As used herein, the term “neuropeptide Y receptor” or “Y receptor” shall be taken to mean a peptide, polypeptide or protein having at least about 80% sequence identity to the amino acid sequence set forth in any one of SEQ D NOs: 2, 4, 6, 8, 10, 12, 14, 16, 18 or 20. Preferably, a neuropeptide Y receptor is selected from the group consisting of a Y1 receptor, a Y2 receptor, a Y4 receptor, a Y5 receptor, a y6 receptor and a Y7 receptor. The term “neuropeptide Y receptor” shall also be taken to mean a peptide, polypeptide or protein that has a ligand selected from the group consisting of neuropeptide Y, peptide YY and pancreatic polypeptide. Preferably, a neuropeptide Y receptor is capable of modulating energy homeostasis of a subject and/or feeding behavior of a subject and/or blood pressure in a host and/or circadian rhythms of a host and/or bone remodeling in a host. Most preferably a neuropeptide Y receptor is capable of modulating bone remodeling in a host. Methods of determining Y receptor activity are known to those skilled in the art and/or described herein.

1. Modulators of Bone Remodeling

In one embodiment, the present invention provides a method for determining a modulator of neuropeptide Y receptor associated bone remodeling:
(v) determining the level of neuropeptide Y receptor associated bone remodeling in the presence of a candidate compound; and
(vi) determining the level of neuropeptide Y receptor associated bone remodeling in the absence of a candidate compound,
   wherein a difference in the level of said neuropeptide Y receptor associated bone remodeling at (i) and (ii) indicates that the candidate compound is a modulator of Y receptor associated bone remodeling.

As will be apparent to the skilled artisan, the process of bone remodeling occurs in mature subjects, that is, this process is the process by which bone is recycled and/or regenerated, rather than the process by which bone is formed in a developing organism.

As used herein, the term “bone remodeling” shall be taken to mean the process by which bone is resorbed by osteoclasts and new areas of bone are deposited by osteoblasts, especially in a mature or post-pubescent subject or a subject that has completed its growth phase. Under normal states of bone homeostasis, the remodeling activities in bone serve to remove bone mass where the mechanical demands of the skeleton are low and form bone at the those sites where mechanical loads are repeatedly transmitted. As will be apparent to the skilled artisan an increase in the number or activity of osteoblasts or a decrease in the number of or activity of osteoclasts results in the formation of more new bone than is resorbed. On the other hand, a decrease in the number or activity of osteoblasts or an increase in the number of or activity of osteoclasts causes more bone to be resorbed than is formed. Accordingly, modulation of bone remodeling facilitates treatment of disorders such as, for example, osteoporosis by inducing bone formation or treatment of disorder such as, for example, osteopetrosis by inducing bone resorption. Methods of determining and/or measuring bone remodeling are known to those skilled in the art and/or described herein.

As will be apparent from the preceding description the term “neuropeptide Y receptor associated bone remodeling” means that bone remodeling (including formation and/or resorption) is controlled by a change in the level of a neuropeptide Y receptor and/or the activity of a neuropeptide Y receptor.

In the present context, the term “modulator” shall be taken to mean a small molecule, nucleic acid, protein or antibody capable of antagonizing or agonizing Y receptor associated bone remodeling selectively or non-selectively. In this regard, the modulator may modulate bone remodeling that is associated with a single Y receptor, or alternatively may modulate bone remodeling activity that is associated with a combination of Y receptors.

In one embodiment, the method of the present invention comprises determining the level that the modulator affects neuropeptide Y receptor activity and determining the level that the modulator affects bone remodeling.

Modulators of Y receptor activity are known in the art and include, for example, the Y2 antagonist T₄-[NPY(33-36)]₄ (Grouzmann et al., Journal of Biological Chemistry, 272(12): 7699-7706, 1997), the Y1 receptor agonist [F7,P34] NPY (Soll et al, Eur. J. Biochem, 268: 2828-2837, 2001), the Y1 receptor antagonist BIBP3326 (available from Bachem Ltd, St Helens, UK), the Y2 receptor agonist Ahx[5-24]NPY (Beck-Sickinger et al., Eur. J. Biochem., 206: 957-604, 1992), Y4 receptor antagonists filipin III and phenylarsine oxide (Parker et al., Eur. J. Pharmacol, 452(3): 279-287, 2002) and the Y receptor inhibitors described in U.S. Pat. No. 6,569,856, U.S. Pat. No. 6,340,683, U.S. Pat. No. 6,225,330, U.S. Pat. No. 6,222,040, U.S. Pat. No. 6,218,408, U.S. Pat. No. 6,214,853 and U.S. Pat. No. 6,124,331.

Alternatively, a compound that modulates Y receptor associated bone remodeling is produced or isolated using conventional means. Preferable compounds include a small molecule, nucleic acid, protein or antibody.

As used herein the term “antibody” refers to intact monoclonal or polyclonal antibodies, immunoglobulin (IgA, IgD, IgG, IgM, IgE) fractions, humanized antibodies, or recombinant single chain antibodies, as well as fragments thereof, such as, for example Fab, F(ab)₂, and Fv fragments.

Antibodies referred to herein are obtained from a commercial source, or alternatively, produced by conventional means. Commercial sources will be known to those skilled in the art.

High titer antibodies are preferred, as these are more useful commercially in kits inhibiting the activity of a protein. By “high titer” is meant a titer of at least about 1:10³ or 1:10⁴ or 1:10⁵.

Antibodies may be prepared by any of a variety of techniques known to those of ordinary skill in the art, and are described, for example in, Harlow and Lane (In: Antibodies: A Laboratory Manual, Cold Spring Harbor Laboratory, 1988). In one such technique, an immunogen comprising the antigenic polypeptide is initially injected into any one of a wide variety of mammals (e.g., mice, rats, rabbits, sheep, humans, dogs, pigs, chickens and goats). The immunogen is derived from a natural source, produced by recombinant expression means, or artificially generated, such as by chemical synthesis (e.g., BOC chemistry or FMOC chemistry). In this step, the Y receptors or fragments thereof described herein may serve as the immunogen without modification. Alternatively, a peptide, polypeptide or protein is joined to a carrier protein, such as bovine serum albumin or keyhole limpet hemocyanin. The immunogen and optionally a carrier for the protein is injected into the animal host, preferably according to a predetermined schedule incorporating one or more booster immunizations, and blood collected from said the animals periodically. Optionally the immunogen may be injected in the presence of an adjuvant, such as, for example Freund's complete or incomplete adjuvant, lysolecithin and dinitrophenol to enhance the immune response to the immunogen. Monoclonal or polyclonal antibodies specific for the polypeptide may then be purified from the blood isolated from an animal by, for example, affinity chromatography using the polypeptide coupled to a suitable solid support.

Monoclonal antibodies specific for the antigenic polypeptide of interest may be prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity (i.e., reactivity with the polypeptide of interest). Such cell lines may be produced, for example, from spleen cells obtained from an animal immunized as described above. The spleen cells are then immortalized by, for example, fusion with a myeloma cell fusion partner, preferably one that is syngenic with the immunized animal. A variety of fusion techniques may be employed, for example, the spleen cells and myeloma cells may be combined with a nonionic detergent or electrofused and then grown in a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and growth media in which the cells have been grown is tested for the presence of binding activity against the polypeptide (immunogen). Hybridomas having high reactivity and specificity are preferred.

Monoclonal antibodies may be isolated from the supernatants of growing hybridoma colonies using methods such as, for example, affinity purification. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The polypeptides of this invention may be used in the purification process in, for example, an affinity chromatography step.

It is preferable that an immunogen used in the production of an antibody is one which is sufficiently antigenic to stimulate the production of antibodies that will bind to the immunogen and is preferably, a high titer antibody. In one embodiment, an immunogen may be an entire protein.

In another embodiment, an immunogen may consist of a peptide representing a fragment of a peptide. Preferably an antibody raised to such an immunogen also recognizes the full-length protein from which the immunogen was derived, such as, for example, in its native state or having native conformation.

Alternatively, or in addition, an antibody raised against a peptide immunogen will recognize the full-length protein from which the immunogen was derived when the protein is denatured. By “denature” is meant that conformational epitopes of the protein are disrupted under conditions that retain linear B cell epitopes of the protein. As will be known to a skilled artisan linear epitopes and conformational epitopes may overlap.

In one embodiment, a peptide immunogen is determined using the method described by Hopp Peptide Research 6, 183-190 (1993), wherein a hydrophilic peptide is selected as it is more likely to occur at the surface of the native protein. However, a peptide should not be too highly charged, as this may reduce the efficiency of antibody generation.

In another embodiment, a peptide immunogen is determined using the method described by Palfreyman et al J. Immunol. Meth. 75, 383-393 (1984), wherein the amino- and/or carboxy-terminal amino acids are used to generate a peptide against which specific antibodies are raised.

In yet another embodiment, a peptide immunogen is predicted using an algorithm such as for example that described in Kolaskar and Tongaonkar FEBS Lett. 276(1-2) 172-174 (1990). Such methods are based upon determining the hydrophilicity of regions of a protein, usually 6 amino acids, and determining those hydrophilic regions that are associated with turns in proteins, surface flexibility, or secondary structures, and are unlikely to be modified at the post-translational level, such as, for example by glycosylation. Such regions of a protein are therefore likely to be exposed, that is, at the surface of the three-dimensional structure of the protein. Furthermore, as these regions are not modified, they are likely to remain constant and as such offer a likely site of antibody recognition.

In yet another embodiment, overlapping peptides spanning the entire protein of interest, or a region of said protein may be generated by synthetic means, using techniques known in the art. Alternatively, a relatively short protein of low abundance or a portion of a protein that is difficult to purify from a natural source, can be produced chemically (e.g. by BOC chemistry or FMOC chemistry).

Synthetic peptides are then optionally screened to determine linear B cell epitopes, using techniques known in the art. In one embodiment, the peptides are screened using an ELISA based screen to determine those against which an immunized subject has raised specific antibodies.

Alternatively, or in addition, such an immunogenic peptide is used to generate a monoclonal or polyclonal antibody using methods known in the art, such as, for example, those described herein. The antibody is then tested to determine its specificity and sensitivity using, for example, an ELISA based assay.

As stated supra a compound may be a ligand of a Y receptor. As used herein the term “ligand” shall be taken in its broadest context to include any chemical compound, polynucleotide, peptide, protein, lipid, carbohydrate, small molecule, natural product, polymer, etc. that is able to bind selectively and stoichiometrically, whether covalently or not, to one or more specific sites on a target (e.g., a protein, carbohydrate, lipid, peptide, macromolecules, biological macromolecules, oligonucleotide, polynucleotide). Preferably, the target is a protein. The ligand may bind to its target via any means including hydrophobic interactions, hydrogen bonding, electrostatic interactions, van der Waals interactions, pi stacking, covalent bonding, or magnetic interactions amongst others.

To identify a suitable ligand, a random peptide library is generated and screened as described in U.S. Pat. Nos. 5,733,731, 5,591,646 and 5,834,318. Generally such libraries are generated from short random oligonucleotides that are expressed either in vitro or in vivo and displayed in such a way that the library may be screened to identify a peptide that is able to specifically bind to a protein or peptide of interest. Methods of display include, phage display, retroviral display, bacterial surface display, bacterial fiagellar display, bacterial spore display, yeast surface display, mammalian surface display, and methods of in vitro display including, mRNA display, ribosome display and covalent display.

A peptide that is able to bind a protein or peptide of interest is identified by a number of methods known in the art, such as, for example, standard affinity purification methods as described, for example in Scopes (In: Protein purification: principles and practice, Third Edition, Springer Verlag, 1994) purification using FACS analysis as described in U.S. Pat. No. 6,455,63, or purification using biosensor technology as described in Gilligan et al, Anal Chem, 74(9): 2041-2047, 2002.

A chemical small molecule library is also clearly contemplated for the identification of a ligand that specifically bind to a Y receptor. Chemical small molecule libraries are available commercially or alternatively may be generated using methods known in the art, such as, for example, those described in U.S. Pat. No. 5,463,564.

In one embodiment, the compound administered comprises nucleic acid, Preferably, the nucleic acid is an antagonist of expression of a Y receptor, such as, for example, an antisense nucleic acid, peptide nucleic acid (PNA), ribozyme, or interfering RNA, which is complementary, in whole or in part, to a target molecule comprising a sense strand, and can hybridize with the target molecule, in particular, a Y receptor-encoding RNA. When introduced into a cell using suitable methods, such a nucleic acid inhibits the expression of the Y receptor gene encoded by the sense strand. Antisense nucleic acid, ribozymes (eg. Cech et al., U.S. Pat. No. 4,987,071; Cech et al., U.S. Pat. No. 5,116,742; Bartel and Szostak, Science 261, 1411-1418, 1993), nucleic acid capable of forming a triple helix (eg. Helene, Anticancer Drug Res. 6, 569-584, 1991), PNAs (Hyrup et al., Bioorganic & Med. Chem. 4, 5-23, 1996; O'Keefe et al., Proc. Natl. Acad. Sci. USA 93, 14670-14675, 1996), interfering RNAs (Elbashir et al., Nature 411, 494-498, 2001; Sharp, Genes Devel. 15, 485-490, 2001; Lipardi et al., Cell 107, 297-307, 2001; Nishikura, Cell 107, 415-418, 2001) or small interfering RNAs (siRNA) may be produced by standard techniques known to the skilled artisan, based upon the sequences disclosed herein.

Preferably, the antisense nucleic acid, ribozyme, PNA, interfering RNA or siRNA comprises a sequence that is complementary to at least about 15-20 contiguous nucleotides of a sequence having at least about 80% identity to SEQ ID NOs: 1, 3, 5, 7, 9, 11, 13, 15, 17, 19 and can hybridize thereto. Longer molecules, comprising a sequence that is complementary to at least about 25, or 30, or 35, or 40, or 45, or 50 contiguous nucleotides of Y receptor-encoding mRNA are also encompassed by the present invention.

A compound that modulates Y receptor mediated bone remodeling may enhance Y receptor mediated bone remodeling or suppress Y receptor mediated bone remodeling. As exemplified herein, suppression of the expression or activity of a Y receptor causes increased bone remodeling. Accordingly, an inhibitor or antagonist of Y receptor expression or activity is an enhancer of Y receptor mediated bone remodeling.

Assays for determining the activity of a Y receptor are known in the art. For example, a modulator of neuropeptide Y activity may enhance or inhibit the binding of a ligand of the receptor (eg neuropeptide Y (NPY), pancreatic polypeptide and/or peptide YY (PYY)), thereby modulating the activation of said receptor. Methods of determining the amount of peptide bound to a receptor are known to those skilled in the art. For example, a cell or isolated membrane comprising at least one Y receptor type is incubated with a test compound and a ligand that has been labeled with a detectable marker, such as, for example, a radioactive marker (eg ¹²⁵I) or a fluorescent marker for a time and under conditions to allow interaction of the Y receptor, the ligand and the compound. Following washing, the amount of labeled ligand bound to the cell or membrane is detected using methods known in the art. An amount of labeled ligand that is bound to a cell or isolated membrane in the presence of a compound to the amount of labeled ligand that is significantly reduced compared to the amount bound to a cell or isolated membrane in the absence of a compound indicates that the compound inhibits the binding of a ligand to a Y receptor.

In one embodiment, Y receptor activity is determined using a linked assay, by measuring cAMP levels in a cell. Activation of a Y receptor has been observed to be associated with coupling to G-proteins which are inhibitory of adenylate cyclase activity, and as a result inhibit forskolin-stimulated cAMP accumulation. Accordingly, a cell that has been exposed to a concentration of a test compound (eg a cell isolated from a subject that has been administered an amount of a test compound) is treated with an amount of forskolin sufficient to induce cAMP accumulation and the amount of cAMP in the cell determined using a radioimmunoassay (as available from Advanced Magnetics, Cambridge, Mass., USA). Alternatively, or in addition, the amount of cAMP induced by forskolin is assessed using a phosphodiesterase kit (available from Molecular Devices, Sunnyvale, Calif., USA) or a phosphodiesterase assay performed in a microplate (as available from Perkin Elmer Life Sciences, Boston, Mass., USA). In accordance with this embodiment, the detection of an amount of cAMP in a cell that as been treated with a test compound that is less than an amount of cAMP detected in a cell that has not been treated with a test compound indicates that the compound inhibits Y receptor activity.

In another embodiment, a method of determining Y receptor involves determining the amount of intracellular free calcium by, for example, microspectrofluorometry.

In accordance with this embodiment, cells isolated from an animal subject (for example, a section of a brain or a hypothalamus) are incubated with a marker that binds to Ca²⁺ by cytoplasmic esterases. Preferably, a marker that is readily detected, such as, for example, a fluorescent marker. For example, a cell sample is incubated with a QUIN 2/AM or fluo-3/AM or a Flura-2/AM fluorescent probe (Calbiochem) for a time and under conditions to facilitate interaction with free intracellular Ca2+. Cells are then washed and the amount of bound probe determined using a confocal microscope and the raw fluorescence data converted to calcium concentrations using standard calcium concentration curves and software analysis techniques, as described in, for example Grouzmann et al., Journal of Biological Chemistry, 272(12): 7699-7706, 1997.

Assays for measuring bone remodeling are known in the art and/or described herein. For example, Mundy et al (Science, 286: 1946-1949, 1999) describe a method of incubating murine calvarial (skullcap) bones in organ culture with a test compound to determine the effect of a test compound on bone remodeling. Sections of the calvarial bone are then analyzed to determine the effect of the compound on parameters such as, bone thickness, formation of new bone, osteoblast numbers, osteoclast numbers, cell proliferation and apoptosis. Preferably, the data determined for each of these parameters are at least compared to a negative control, such as, for example a calvarial bone that is not incubated in the presence of a test compound. Additionally, the data derived for each for the parameters may be compared to data derived from a positive control, such as, for example, a calvarial bone incubated in the presence of fibroblast growth factor.

In another embodiment, bone remodeling is assessed by determining the rate of bone resorption using an assay essentially as described by Shigeno et al, J. Clin. Endocrinol. Metab., 61: 761-768, 1985 and Sone et al, Endocrinology, 131: 2742-2746, 1992. Briefly, bone resorption is assessed by determining the amount previously incorporated ⁴⁵Ca that is released from the calvariae of an animal subject. A neonatal animal subject is injected subcutaneously with an amount of ⁴⁵Ca (in the form of CaCl₂). Following a sufficient time (ie. about 2 days), calvarial bone is isolated from the neonatal animal and cultured in an organ culture in the presence or absence of a test compound for a time sufficient to determine the effect of the compound on bone remodeling. Media is then isolated from the culture and the amount of radioactivity (⁴⁵Ca) that has been released into the media is determined. Alternatively, or in addition, the amount of ⁴⁵Ca remaining in the bone is determined. A difference in the amount of ⁴⁵Ca detected in the bone or media of a culture incubated in the presence of a test compound compared to the amount of ⁴⁵Ca detected in a culture incubated with no compound indicates that the compound modulates bone remodeling.

In a still further embodiment, bone remodeling is determined by incubating a calvarial bone isolated from a neonatal or fetal animal subject in an organ culture comprising ³H-proline and/or ⁴⁵Ca. As new collagenous matrix is formed by osteoblast activity ³H-proline is incorporated into the matrix. Furthermore, as mineralization occurs calcium (and thereby ⁴⁵Ca) is deposited at regular intervals along the longitudinal axis of the collagenous matrix. Media is then isolated from the culture and the amount of radioactivity (³H-proline and/or ⁴⁵Ca) that remains in the media is determined. Alternatively, or in addition, the amount of ³H-proline and/or ⁴⁵Ca in the bone is determined. A difference in the amount of ³H-proline and/or ⁴⁵Ca detected in the bone or media of a culture incubated in the presence of a test compound compared to the amount of ³H-proline and/or ⁴⁵Ca detected in a culture incubated with no compound indicates that the compound modulates bone remodeling.

In a particularly preferred embodiment, the effect of a modulator on bone remodeling is assessed in an animal model. In accordance with this embodiment various parameters may be determined. For example, cortical area is determined by bisecting a bone from an animal subject (such as, for example, a femur) and subtracting the medullary area from the total bone area. Cortical thickness is calculated by circumference difference of the total bone area and medullary area of a bisected bone. Transversely bisected bones are stained for mineralized bone and trabecular bone volume, thickness and number are assessed using methods known in the art and described, for example, in Parfitt et al., J. Clin. Invest., 72: 1396-1409, 1983. Mineralization rate and bone formation rate are determined using fluorescence microscopy of bone sections using methods known in the art and described, for example, in Parfitt et al, J. Bone Mineral. Res., 2: 595-610, 1987.

A preferred embodiment of the present invention provides a method of determining a modulator of bone remodeling comprising administering to an animal subject having wild-type bone remodeling activity an amount of a candidate compound and determining the Y receptor and bone remodeling activity of the animal subject wherein a modified level of Y receptor activity and bone remodeling in the presence of the compound to the activities in the absence of the compound indicates that the compound is a modulator of Y receptor associated bone remodeling.

As used herein, the term “wild-type bone remodeling activity” shall be taken to mean that an animal subject displays the same degree of bone remodeling (ie. bone formation and/or bone resorption) as an animal that has not been treated with a compound that may modulate bone remodeling or modified at the genetic level (ie. a “knockout” mouse) to silence a gene that may be associated with bone remodeling. However, an animal may have been treated with a compound, or lack expression of a gene that does not alter the remodeling activity of that animal from that of wild-type bone remodeling activity. Alternatively, the subject animal may express a heterologous gene that does not alter the remodeling activity of that animal from that of wild-type bone remodeling activity. Such an animal, may, for example, be sensitive to compounds that modulate bone remodeling activity.

When used in the context of cells grown in vitro, the term “wild-type bone remodeling activity” shall be understood to include the ability of a cell to form a mineralized matrix or any stage leading thereto.

In one embodiment, an animal with “wild-type bone remodeling activity” is a wild-type animal. By “wild-type” is meant that an organism has the phenotype of the majority of organisms of that group (eg species, subspecies or strain) that occur naturally, ie. those organisms that are not mutants or have not been treated with a compound.

In another embodiment, an animal with “wild-type bone remodeling activity” is a mutant animal that retains wild-type bone remodeling activity. By “mutant animal” is meant an animal in which a genetic change has been induced (eg by homologous recombination to silence a gene or by transgenesis) that modulates the expression of one or more genes. Such genetic changes may or may not be inheritable, for example the genetic change may be post-transcriptional silencing of a gene using, for example RNAi. Methods of producing such mutant animals are known in the art and/or described herein. In accordance with this embodiment, the method of the present invention may comprise the additional step of providing an animal with wild-type bone remodeling activity.

In one embodiment, an animal with wild-type bone remodeling activity is a mutant animal whereby the mutation causes said animal to be sensitive to a modulator of bone remodeling. A mutant animal that is sensitive to a modulator of bone remodeling is an animal that shows a change in a bone remodeling phenotype when exposed to about half the concentration of a modulator required to induce the bone remodeling phenotype in a wild-type animal. Preferably, the concentration of the modulator is about one quarter that required to induce a phenotype in a wild-type organism, more preferably one tenth, even more preferably one hundredth.

In one embodiment, an animal with wild-type bone remodeling activity is any vertebrate model organism. Preferably, the organism expresses at least one Y receptor. Even more preferably, bone remodeling in such organisms is associated with expression of at least one Y receptor. A model organism that is known to express at least one Y receptor is an animal selected from the group consisting of a rat, a mouse, a chimpanzee, chicken, a guinea pig, a rabbit, a bovine species, a sheep, and a zebrafish.

In a particularly preferred embodiment, the animal subject is a mouse. Preferably, a strain of laboratory mouse.

In a preferred embodiment, an animal with wild-type bone remodeling activity is a mouse. Wild-type mice express several forms of Y receptor (ie. at least Y1 receptor, Y2 receptor, Y4 receptor, Y5 receptor and y6 receptor). As exemplified herein, Modulation of Y receptor activity in mice results in changes in bone morphology as a result of changed bone remodeling that closely mimics the changes in humans.

Furthermore, the physiology of mice is relatively well characterized. Accordingly, a modulator that specifically changes Y receptor associated bone remodeling in a mouse is most likely to specifically modulate Y receptor associated bone remodeling in a human.

In one embodiment, a mouse that is deficient in a Y receptor is generated using RNAi, siRNA, PNA or a ribozyme. Preferably, the antisense nucleic acid, ribozyme, PNA, interfering RNA or siRNA comprises a sequence that is complementary to at least about 15-20 contiguous nucleotides of a sequence having at least about 80% identity to SEQ ID NOs: 1, 3, 5, 7, 9, 11, 13, 15, 17 or 19 and can hybridize thereto. Longer molecules, comprising a sequence that is complementary to at least about 25, or 30, or 35, or 40, or 45, or 50 contiguous nucleotides of a Y receptor-encoding mRNA are preferred.

In another embodiment, a mouse that is deficient in a Y receptor is a mouse that comprises a gene construct that expresses a double-stranded RNA molecule that is complementary to at least about 15-20 contiguous nucleotides of a sequence having at least about 80% identity to SEQ ID NOs: 1, 3, 5, 7, 9, 11, 13, 15, 17 or 19. Such molecules are capable of inducing RNA mediated silencing (by cleavage of the expression product of a Y4 receptor gene) of a Y4 receptor gene. Methods of producing such constructs are known in the art and described, for example in Calegari et al, Proc. Natl. Acad. Sci. USA, 99(22): 14236-14240, 2002 and Caplen et al, Proc. Natl. Acad. Sci. USA, 98(17: 9742-9747, 2001.

In a particularly preferred embodiment, a mouse that is deficient in a Y receptor is a mouse in which a gene encoding a Y receptor has been silenced by targeted disruption (ie. a knockout mouse). Methods of disrupting a gene are known in the art and are described, for example, in Nagy et al eds. Manipulating the Mouse Embryo, Cold Spring Harbor Laboratory, 3rd Edition, 2002, ISBN 0879695749 and Tymms and Kola eds Gene Knockout Protocols, Humana Press, 2001, ISBN: 0896035727. Briefly, a targeting construct is produced that comprises two regions (arms) that are homologous to a region of a Y encoding gene. Such a construct further comprises a region, or cassette, that encodes a selectable marker such as, for example, a neomycin resistance gene or a β-galactosidase gene, or a combination of a neomycin gene and a β-galactosidase gene (ie a β-geo gene), that is located between the two arms of the construct. The gene construct is then transfected or electroporated into embryonic stem cells, which are incubated for a time and under conditions to select for cells that have incorporated the gene construct into their genome (such as, for example, with G418). Cells that have successfully incorporated the targeting construct into the correct genomic location (ie a Y receptor encoding gene) are selected using methods known in the art (eg Southern blotting, PCR or if the targeting construct comprised the relevant selectable marker/s, using negative selection). Cells that have successfully homologously recombined the targeting construct into a Y receptor encoding gene are microinjected into a blastocyst stage embryo, which is in turn transferred into a female, preferably a pseudopregnant female. Progeny are screened for the targeting construct and those that carry the construct are bred for several generations to produce homozygous knockout mice.

Even more particularly preferred are mice in which a Y receptor is conditionally silenced. By “conditionally silenced” is meant that the silencing of a Y receptor is dependent upon an external stimulus that may be spatially and/or temporally controlled. For example, a Y receptor gene or a region thereof may be flanked by loxP or frt sequences that are arranged in such a way that they are considered to be “in parallel”, using methods for homologous recombination known in the art and/or described herein. Expression of the recombination enzyme Cre recombinase (in the case of loxP sites) or Flp recombinase (in the case of frt) causes the region between the loxP or flp sequences to be excised thereby silencing the Y receptor gene. As exemplified herein, a mouse comprising a Y2 receptor gene flanked by loxP sites (ie floxed) is injected, into the hypothalamus with an adenovirus expressing Cre. Subsequently, Y2 receptor expression is silenced in the hypothalamus.

As will be apparent to the skilled artisan, the methods described herein may be used to silence or conditionally silence any Y receptor gene or any combination of Y receptor genes.

Preferred mice in which a Y receptor gene has been silenced or conditionally silenced are selected from the group consisting of Y1^{-/-}, Y1^{-/+}, Y1^{lox/+}, Y1^{lox/lox}, Y1^{frt/+}, Y1^{frt/frt}, Y2^{-/-}, Y2^{-/+}, Y2^{lox/+}, Y2^{lox/lox}, Y2^{frt/+}, Y2^{frt/frt}, Y4^{-/-}, Y4^{-/+}, Y4^{lox/+}, Y4^{lox/lox}, Y4^{frt/+}, Y4^{frt/frt}, y6^{-/-}, y6^{-/+}, y6^{lox/+}, y6^{lox/lox}, y6^{frt/+}, y6^{frt/frt}, Y7^{-/-}, Y7^{-/+}, Y7^{lox/+}, Y7^{lox/lox}, Y7^{frt/+}, Y7^{frt/frt}, or any combination thereof. As used herein, the term “−/−” shall be understood to symbolize a mouse that is homologous for a mutation that silences a gene; “−/+” shall be taken to symbolize a mouse that is heterozygous for a mutation that silences a gene; “lox/+” shall be taken to symbolize a mouse that is heterozygous for a gene or region thereof that is flanked by loxP sites; “lox/lox” shall be taken to symbolize a mouse that is homozygous for a gene or region thereof that is flanked by loxP sites; “frt/+” shall be taken to symbolize a mouse that is heterozygous for a gene or region thereof that is flanked by frt sites; and “frt/frt” shall be taken to symbolize a mouse that is homozygous for a gene or region thereof that is flanked by frt sites.

Methods of producing mice that are deficient (or conditionally deficient, or a combination of germline deficient and conditionally deficient) will be apparent to the skilled artisan. For example, a mouse that is deficient for one Y receptor gene (eg Y2−/−) may be crossed with a mouse deficient for another Y receptor gene (eg Y4−/−). Alternatively, a stem cell may be transfected with a gene construct for targeting one Y receptor gene, and following successful selection, transfected with a gene construct for targeting another Y receptor. Cells that successfully target both Y receptor genes are then used to produce mice. As will be apparent to the skilled artisan, the choice of selectable markers is important in such experiments.

When determining a modulator of Y receptor associated bone remodeling a model that conditionally silences a Y receptor is preferred. This is because, the process of bone remodeling occurs following bone formation. Accordingly, it is preferred that a Y receptor is not silenced until after the completion of bone remodeling. Methods of conditional or targeted gene silencing known to those skilled in the art include, for example, injection of a virus (eg an adenovirus) that expresses the relevant recombination enzyme or inducible expression of the relevant recombinase using an inducible promoter, such as, for example a promoter that is suppressed by the administration of tetracycline.

In one embodiment, a Y receptor is silenced in a mouse following the completion of bone development, ie after approximately 8 weeks of age.

In another embodiment, a test compound is not administered to an animal subject until after the completion of bone development.

In one embodiment, the method of the present invention provides a method of determining a modulator of bone remodeling comprising administering to an aged animal subject having wild-type bone remodeling activity an amount of a candidate compound and determining the Y receptor and bone remodeling activity of the aged animal subject wherein a modified level of Y receptor activity and bone remodeling in the presence of the compound to the activities in the absence of the compound indicates that the compound is a modulator of Y receptor associated bone remodeling.

Preferably, a Y receptor is silenced and/or a test compound is administered to an aged animal subject. Accordingly, a compound that is capable of modulating bone remodeling activity in an aged animal subject provides a candidate compound for modulation of bone remodeling in an aged subject. As many bone diseases of humans are associated with ageing, eg osteoporosis, as bone development begins to decline after approximately 35 years of age in a human. In accordance with this embodiment, an aged model organism is a mouse that is aged for approximately 8 weeks, more preferably for about 36 weeks and even, more preferably about 48 weeks.

As exemplified herein, silencing of a Y2 receptor in an aged animal subject (ie a 16 week old mouse) induces the process of bone remodeling, whereby the amount of bone in the animal is increased. Accordingly, a compound that is capable of suppressing the expression and/or activity of a Y receptor is capable of modulating bone remodeling in a subject.

Another embodiment of the present invention provides a method of determining a modulator of bone remodeling comprising administering to an animal subject having osteoporotic bone remodeling activity an amount of a candidate compound and determining the Y receptor and bone remodeling activity of the animal subject wherein a modified level of Y receptor activity and bone remodeling in the presence of the compound to the activities in the absence of the compound indicates that the compound is a modulator of Y receptor associated bone remodeling.

A high incidence of osteoporosis is observed in post-menopausal women or in males suffering from reduced levels of circulating sex hormones, suggesting that sex hormones play an important role in the development of osteoporosis. An important animal model of osteoporosis is a mouse that has been gonadectomized. Such mice show a very similar phenotype to humans that suffer from osteoporosis with dramatically weakened bones and an increase in the number of bone fractures. Accordingly, in one embodiment, a modulator of bone remodeling activity is determined in an animal subject that has been gonadectomized in order to induce an osteoporotic phenotype.

In a particularly preferred embodiment a mouse that is used to determine a modulator of bone remodeling is a mouse in which at least a portion of the coding region of the Y4 receptor of the mouse has been excised or disrupted. Such Y4 deficient mice display wild-type bone remodeling activity, however are particularly sensitive to changes that modulate Y receptor associated bone remodeling. For example, inhibition of Y2 receptor activity in a Y4 receptor deficient mouse induces dramatically increased bone volume when compared to a wild-type mouse or a mouse in which Y2 receptor activity has been inhibited.

In one embodiment, the present invention provides a method for determining a modulator of neuropeptide Y receptor associated bone remodeling comprising administering to a Y4 receptor deficient animal subject an amount of a candidate compound and determining the bone remodeling activity of the animal subject wherein a difference in the level of said bone remodeling activity in the presence of the compound compared to the level of said bone remodeling activity in the absence of the compound indicates that the compound is a modulator of neuropeptide Y receptor associated bone remodeling.

One embodiment of the present invention provides a method for determining a modulator of neuropeptide Y receptor associated bone remodeling comprising administering a modulator of Y receptor activity to an animal subject and determining a bone remodeling phenotype, wherein a modified bone phenotype in the presence of the compound compared to the bone phenotype in the absence of the compound indicates that the compound is a modulator of Y receptor associated bone remodeling.

In accordance with this embodiment, the modulator of Y receptor activity may be a known modulator of Y receptor activity as described supra or alternatively the modulator of Y receptor activity may be a modulator identified or produced using the methods described supra.

A still further embodiment of the present invention provides a method for determining a modulator of neuropeptide Y receptor associated bone remodeling comprising administering a modulator of bone remodeling to an animal subject and determining a change in Y receptor activity, wherein a modified Y receptor activity in the presence of the compound compared to the Y receptor activity in the absence of the compound indicates that the compound is a modulator of Y receptor associated bone remodeling.

In accordance with this embodiment, the modulator of bone remodeling may be a known modulator of bone remodeling, such as, for example, Simvastatin, Lovastatin, estrogen, Raloxifene, bisphosphates, calcitrol, or analogues of calcitrol, such as, for example alphacalcitrol, or a modulator of bone remodeling identified using the methods described supra.

While any molecule that modulates Y receptor activity is sufficient to modulate bone remodeling, a molecule that modulates Y receptor activity and is incapable of passing through the blood brain barrier or crosses the blood brain barrier inefficiently or at a level that is inefficient to permit one or more undesirable Y receptor-mediated effects is preferred. As would be understood by the skilled artisan, an undesirable Y receptor mediated effect is an effect mediated by a Y receptor signaling pathway within the blood brain barrier eg., an effect other than bone remodeling, bone growth, adiposity, or differentiation of a stem cell into an osteoblast. This is because, as shown by Y receptor null mutant mice, elimination of Y receptors causes changes in a wide variety of physiological processes. As exemplified herein, the process of bone remodeling in a subject is controlled by Y receptors expressed in the arcuate nucleus or in other regions of the body, but not in those areas of the brain contained within the blood brain barrier. As used herein, the term “blood brain barrier” shall be taken to mean an anatomical-physiological feature of the brain that is thought to consist of walls of capillaries in the central nervous system and surrounding glial membranes. The barrier separates the parenchyma of the central nervous system from blood. The blood-brain barrier functions in preventing or slowing the passage of various chemical compounds, radioactive ions, and disease-causing organisms, such as viruses, from the blood into the central nervous system. In order to pass across the blood brain barrier a molecule should be less than about 400-600 daltons and lipid soluble, or alternatively, linked to a molecule that passes across the blood brain barrier. Accordingly, such molecules are selected against in the methods of the present invention.

In one embodiment, preferred modulators of Y receptor mediated bone remodeling are modulators that cannot pass across the blood brain barrier. In accordance with this embodiment, peptides, antibodies, siRNA, PNA, RNAI, ribozymes and antisense molecules are preferred test compounds, as unless modified to do so these molecules cannot readily pass across the blood brain barrier.

Assays for determining whether or not a compound that modulates Y receptor associated bone remodeling is capable of passing across the blood brain barrier are apparent to the skilled artisan. For example, a compound that is capable of modulating Y receptor activity may be administered to an animal subject and the subject monitored not only for changes that indicate a change in bone remodeling but also phenotypic changes that are associated with Y receptor activity in regions of the brain protected by the blood brain barrier. For example, a mouse may be analyzed to determine a change in body fat content (for example by weighing an inguinal, an epididymal, a retroperitoneal, and/or a mesenteric fat pad), comparing brown adipose tissue and white adipose tissue levels, determining bodyweight, determining food intake (either normal feeding, or feeding after forced fasting) or determining activity levels of a mouse.

Alternatively, a biological sample is derived from a region of the body that is encompassed by the blood brain barrier, such as, for example brain tissue or cerebrospinal fluid. Such a biological tissue is then analyzed to detect the presence of a compound that has been administered to the subject. As will be apparent to the skilled artisan a method of determining the presence of a compound in a biological sample is dependent upon the compound in question.

In another embodiment, a method of determining a compound that is capable of crossing the blood brain barrier and modulating Y receptor activity is determined by ascertaining the activity of a Y receptor in a region of the body that is encompassed by the blood brain barrier. Methods of determining Y receptor activity are known to those skilled in the art and/or described herein. In accordance with this embodiment, a modulator that is capable of modulating the activity of a Y receptor in a region that is encompassed by the blood brain barrier is selected against.

In a further embodiment, the assay of the present invention comprises the additional step of determining a phenotype other than bone remodeling in an animal subject that has been exposed to an amount of a candidate compound wherein a modified level of the phenotype in the presence of the compound compared to the phenotype in the absence of the compound indicates that the compound is not a specific modulator of Y receptor associated bone remodeling.

Preferred modulators of bone remodeling are those modulators that specifically modulate bone remodeling. As described supra Y receptors modulate a wide variety of physiological processes. Accordingly, a modulator that is not bone remodeling specific may modulate activity levels, sleep patterns, memory, feeding behavior and body fat levels. Such a compound is clearly not preferred for the treatment of a bone disorder in a human subject in need of treatment.

Another embodiment of the present invention provides a method of determining a compound that modulates the bone remodeling activity of a specific Y receptor. In accordance with this embodiment, an animal that is deficient in the specific Y receptor activity of interest is produced or provided (for example an animal that is a conditional knockout of a Y receptor, wherein the Y receptor is only knocked-out in those tissues required to produce a change in Y receptor associated bone remodeling) as described supra. Following production or provision of such an animal, the bone of the animal is characterized using, for example gene profiling analysis or protein expression analysis to determine those changes that are associated with a change in Y receptor associated bone remodeling. A candidate compound is subsequently screened to determine whether or not it is capable of inducing gene expression changes in a bone that are associated with Y receptor associated bone remodeling.

The effect of a Y receptor on the expression of gene products associated with bone remodeling is ascertained by determining the gene product expression profile of a bone derived from an animal in which at least one Y receptor has been silenced and comparing this to the gene product expression profile of a bone derived from a wild-type animal. By “gene product” is meant any transcription product of a genomic gene, such as unprocessed or processed mRNA including a splice variant, or any translation product encoded by a genomic gene, such as a precursor polypeptide, processed polypeptide or a complex involving said polypeptide. The term “gene product expression profile” shall be taken to mean the characterization of the expression level of at least one gene product. Preferably, a gene expression profile encompasses the expression level of several gene products. Methods of determining a gene expression profile are known to those skilled in the art and include, a DNA microarray, an antibody array or 2-dimensional protein electrophoresis.

In one embodiment a candidate compound is screened by administration to an animal with wild-type bone remodeling activity for a time and under conditions to allow the compound to affect the bone remodeling of the animal or the expression of genes associated with bone remodeling in the animal. Bone tissue is subsequently isolated from the animal subject and the expression product profile of the bone determine. A modulator that is capable of inducing a similar gene expression profile to the Y receptor deficient mouse bone is a modulator capable of modulating Y receptor associated bone remodeling. Preferably, the modulator is not capable of crossing the blood brain barrier and specifically modulates Y receptor mediated bone remodeling.

As used herein the term “a similar gene expression profile” shall be taken to mean that at least 80% of the gene products that are induced and/or suppressed by deficiency of a Y receptor are induced and/or suppressed by the compound. Alternatively, or in addition, a similar gene expression profile may encompass the degree to which the gene product expression is induced or suppressed.

In another embodiment, a candidate compound is screened by administration to a bone cell line, such as, for example 2T3 or MG-63 cells or cultured bone and the expression product profile determined using methods known in the art. A modulator that is capable of inducing a similar gene expression profile to the Y receptor deficient mouse bone is a modulator capable of modulating Y receptor associated bone remodeling.

Preferably, the modulator is not capable of crossing the blood brain barrier and specifically modulates Y receptor mediated bone remodeling.

2. Modulators of Bone Growth

In another embodiment, the present invention provides a method for determining a modulator of neuropeptide Y receptor associated bone growth comprising:
(i) determining the level of neuropeptide Y receptor associated bone growth in the presence of a candidate compound; and
(ii) determining the level of neuropeptide Y receptor associated bone growth in the absence of a candidate compound,
   wherein a difference in the level of said neuropeptide Y receptor associated bone growth at (i) and (ii) indicates that the candidate compound is a modulator of Y receptor associated bone growth.

As used herein, the term “bone growth” shall be taken to mean the process by which bones are generated de novo in a developing or immature organism. Bone growth commences early in embryonic development with the growth of a cartilage model of much of the skeleton by osteoblasts. As the fetus develops, long bones begin to form, a rim of primitive bone is first formed in layers over the middle of the shaft by osteoblasts arising from the overlying periosteum, and subperiosteal bone formed in this way soon extends up and down the shaft (diaphysis). The process of intramembranous ossification then replaces membranous fibrous tissue with mineralized bone tissue and continues to thicken the bone throughout postnatal growth. The cartilage cells of the core of the fetal shaft degenerate upon contact with penetrating buds of periosteal osteoblasts, the cartilage matrix becomes mineralized and resorbed, and the resulting surfaces and spaces are lined by osteoblasts which lay down woven bone and form primitive bone trabeculae. Some of the trabeculae fuse with the subperiosteal new bone while others are resorbed to form a medullary cavity which will be occupied by hematopoietic tissue. As the fetus develops the woven bone of the diaphysis will be replaced by lamellar bone of mature type.

As the process of bone growth occurs in a developing organism, ie from the time of embryogenesis until about the commencement of bone remodeling, the modulation of bone growth is particularly relevant to the treatment of subjects within this developmental window. For example, in a mouse, it is preferred that a method for determining a compound that modulates bone growth is performed prior to the attainment of sexual maturity ie. at less than about 8 weeks of age, and more preferably at about 4-8 weeks of age, and even more preferably, at about 4-6 weeks of age. The skilled artisan will readily be in a position to determine similar developmental windows for other animal models of bone growth.

A compound that modulates Y receptor mediated bone growth may enhance Y receptor mediated bone growth or suppress Y receptor mediated bone growth. As exemplified herein, suppression of the expression or activity of a Y receptor causes increased bone growth. Accordingly, an inhibitor or antagonist of Y receptor expression or activity is an enhancer of Y receptor mediated bone growth.

Methods of determining a modulator of a Y receptor activity or expression as described above apply mutatis mutandis to this embodiment of the invention. Furthermore, methods of identifying a modulator of bone remodeling apply mutatis mutandis to a method of determining a modulator of bone growth, with the proviso that the methods are performed in an animal that is still undergoing bone growth. As will be apparent to the skilled artisan, bone growth is preferably determined by measuring trabeculae volume, trabeculae number, mineral apposition rate, cancellous bone volume, or bone length (eg., femur length) in an immature animal or human subject.

Methods of producing an animal that is deficient or conditionally-deficient in the expression and/or activity of a Y receptor described supra are described herein above. In accordance with this embodiment, it is particularly preferred that a Y receptor gene is silenced during the period of bone growth. Accordingly, a germ line deficient mouse is preferred for assays of modulators of bone growth. As used herein, the term “germ line deficient” shall be taken to mean that a mutation is inheritable and as such affects an animal subject from the time that the expression of the gene occurs in a wild-type animal. A germ line deficient animal need not necessarily lack expression of the Y receptor of interest in all tissues, for example a mouse may carry a “floxed” Y receptor gene and a Cre recombinase under control of a tissue specific promoter. Accordingly, only cells that express Cre lack Y receptor gene expression.

Preferably, germline deficiency of a Y2 receptor, a Y1 receptor, a Y4 receptor, a Y5 receptor, a y6 receptor, a Y7 receptor and/or a combination of these receptors results in enhanced bone growth. Compounds that modulate bone growth and suppress the expression and/or activity of a Y receptor are selected.

One embodiment of the present invention provides a method of determining a modulator of bone growth comprising administering to an animal subject having wild-type bone growth activity an amount of a candidate compound and determining the Y receptor and bone growth activity of the animal subject wherein a modified level of Y receptor activity and bone growth in the presence of the compound to the activities in the absence of the compound indicates that the compound is a modulator of Y receptor associated bone growth.

In one embodiment, a mouse with wild type bone growth activity is a wild type mouse. In another embodiment, a mouse with wild type bone growth activity is a mutant mouse, such as a mouse described supra. In a particularly preferred embodiment, a mutant mouse is a mouse that is deficient in the expression and/or activity of a Y4 receptor is used due to its enhanced sensitivity to reduce activity or expression of other Y receptors, in particular Y1 or Y2.

In accordance with either of the two previous embodiments, a candidate compound is administered to an animal subject prior to the onset of bone remodeling in the animal.

In one embodiment, an animal subject is a mouse. In accordance with this embodiment, a candidate compound is administered to a mouse prior to the onset of bone remodeling, ie prior to the attainment of sexual maturity eg., at about 8 weeks of age, more preferably at about 6 weeks of age, or even more preferably, at about 5 weeks of age. To determine the effect of a candidate compound on bone development, it is preferred that the compound is administered for a time and under conditions that permit the compound to modulate bone growth. Accordingly, in one embodiment, a candidate compound is administered to a mouse that is less than about 8 weeks of age, preferably less than about 4 weeks of age, more preferably less than about 2 weeks of age and even more preferably less than about 1 week of age.

In an alternate embodiment, a candidate compound is administered to a pregnant female animal subject and for a time and under conditions for the compound to modulate Y receptor associated bone growth in a developing embryo. Preferably the effect of the compound on Y receptor activity and bone growth are assessed as described supra. In this regard, an assay to determine the effect of a compound on bone growth is essentially the same as an assay to determine the effect of a compound on bone remodeling.

In one embodiment, a candidate compound is administered to a pregnant animal subject that is heterozygous for a mutation (such as for example a Y4^{-/+} mouse) that has been mated with an animal subject that is heterozygous for the same mutation. As a result of such a breeding strategy the compound will effectively be administered to a wild-type mouse, a heterozygous mutant mouse and a homozygous mutant mouse, thereby determining the effect of the compound on bone growth in each type of animal subject.

As described supra, Y receptors modulate and/or mediate a wide variety of physiological processes via their expression and activity in the regions of the brain encompassed by the blood brain barrier. Accordingly, in a preferred embodiment, a candidate compound that is capable of modulating Y receptor associated bone growth is not capable of crossing the blood brain barrier. Accordingly, the administration of such a compound will not result in the modulation of Y receptor activity in a region of the brain encompassed by the blood brain barrier. Methods for determining a compound that is not capable of crossing the blood brain barrier are known in the art and/or described herein.

In a further preferred embodiment, a candidate compound specifically modulates bone growth activity. As used herein, the term “specifically modulates bone growth activity” shall be taken to mean that a candidate compound in not capable of significantly altering physiological processes other than the growth of bone. Such a compound is particularly preferred for the treatment of disorders associated with aberrant bone growth, as the compound will not affect those processes not associated with this disorder.

In one embodiment, a candidate compound is capable of modulating bone remodeling and bone growth.

A still further embodiment of the present invention provides a method of determining a compound that modulates the bone growth activity of a specific Y receptor comprising, determining the gene expression product profile of a bone derived from an animal subject that is deficient for at least one Y receptor, and determining a compound capable of inducing similar changes in a bone derived from an animal subject that has been administered with the compound.

Preferably, the animal subject to which the compound is administered is an animal with wild-type bone growth activity.

Methods of determining the gene expression product profile are known in the art, and/or described herein. Exemplary methods include transcript profiling such as, for example, by RT-PCR, quantitative PCR, Northern hybridization or dot blot analysis.

In one particularly preferred embodiment, the present invention excludes methods for determining modulators of bone remodeling or growth that are associated with activity or expression of a Y2 receptor and no other Y receptor (i.e. Y2-only associated bone remodeling or bone growth).

3. Modulators of Adiposity

As exemplified herein, mice that are deficient in Y receptor activity show increased bone remodeling activity and/or bone growth activity. Interestingly, these mice also display reduced adiposity. As used herein, the term “adiposity” shall be understood as a measure of the amount of fat, or amount of white adipose tissue or the amount of white adipose tissue relative to brown adipose tissue or the number of adipose cells found in a tissue or subject. Accordingly, an animal subject that shows reduced adiposity, such as, for example a Y2^{-/-} mouse, have a reduced amount of white adipose tissue when measured as a percentage of total body mass or an absolute weight.

Cells isolated from bone marrow stroma (and in particular mesenchymal stem cells (MSCs)) are capable of differentiating into adipocytes, chondrocytes and osteoblasts. Accordingly, as Y receptor deficient mice demonstrate significant increases in bone formation (ie an activity associated with osteoblasts) and a significant reduction in adiposity, a modulator of Y receptor activity also modulates the differentiation of SCs and/or bone marrow stromal cells.

As used herein the term, “mesenchymal stem cell” or “MSC” shall be taken to mean a stem cell, or progenitor cell that is capable of differentiating to form an adipocyte and/or a chondrocyte and/or an osteoblast and/or a myoblast in vitro. Such cells may be isolated from bone marrow stroma or adipose tissue.

In one embodiment, a compound that modulates the differentiation of MSCs and/or bone marrow stromal cells induces the formation of osteoblast cells. Preferably, such a compound also induces bone formation and or bone growth in vivo. Accordingly, such a compound is of particular use in the treatment of one or more bone disorders, such as, for example, osteoporosis. This is because osteoblast number and/or activity is associated with the formation of new bone.

In another embodiment, a compound that modulates the differentiation of MSCs and/or bone marrow stromal cells suppresses the formation of adipocytes. Preferably, such a compound also reduces adiposity in vivo. In accordance with this embodiment, such a compound is of particular use in the treatment of disorders such as, for example, obesity.

In a preferred embodiment, a compound that modulates the differentiation of MSCs and/or bone marrow stromal cells induces the formation of osteoblast cells and suppresses the formation of adipocytes. Alternatively, such a compound induces the formation of bone and reduces the adiposity of an animal subject.

In one embodiment, the present invention provides a method of determining a compound that is a modulator of Y receptor associated differentiation of an osteoblast-type cell comprising administering a candidate compound to a cell, tissue or animal subject an amount of a compound for a time and under conditions to facilitate osteoblast differentiation and determining the level of osteoblast type cell differentiation, wherein a modified level of Y receptor associated osteoblast type cell differentiation in the presence of a compound compared to the level of Y receptor associated osteoblast type cell differentiation in the absence of a compound indicates that the compound is a modulator of Y receptor associated differentiation of an osteoblast type cell.

Preferably, the compound is capable of inducing osteoblast type cell differentiation. Even more preferably the compound is capable of inducing osteoblast cell differentiation to the same degree as bone morphogenetic protein-2 (BMP-2). In this regard, BMP-2 has been shown to be a potent inducer of osteoblast differentiation from MSC cells (Dragoo et al., J. Orthop. Res., 21(4): 622-629, 2003).

As used herein, the term “osteoblast type cell” shall be taken to mean a cell that closely resembles an osteoblast cell at the morphological and biochemical level. Methods of determining an osteoblast type cell are known to those skilled in the art and/or described herein. For example, a cell is monitored for the induction of osteoblast markers, such as for example alkaline phosphatase activity (as described in Pittenger et al, Science, 284: 143-147, 1999), von Kossa staining to detect calcium accumulation in cells (as described in Pittenger et al, Science, 284: 143-147, 1999) and/or the detection of osteogenic differentiation markers cbfa-1/OSF-2 and osteoclacin (essentially as described in Alejandro et al, Exp. Cell Res., 280: 24-32, 2002).

In another embodiment, the present invention provides a method of determining a compound that is a modulator of Y receptor associated differentiation of an adipocyte-type cell comprising administering a candidate compound to a cell, tissue or animal subject an amount of a compound for a time and under conditions to facilitate adipocyte differentiation and determining the level of adipocyte type cell differentiation, wherein a modified level of Y receptor associated adipocyte type cell differentiation in the presence of a compound compared to the level of Y receptor associated adipocyte type cell differentiation in the absence of a compound indicates that the compound is a modulator of Y receptor associated differentiation of an adipocyte type cell.

Preferably, the compound is capable of suppressing adipocyte type cell differentiation.

As used herein, the term “adipocyte type cell” shall be taken to mean a cell that closely resembles an adipocyte cell at the morphological and/or biochemical level. Methods of determining an adipocyte type cell are known to those skilled in the art and/or described herein. For example, a cell is stained with a lipid soluble stain, such as, for example, Oil red-O using methods known in the art, or the expression of adipocyte markers SCD, FAS, aFABP and/or PPARy using methods known in the art, such as, for example, PCR or Northern blotting.

In a preferred embodiment, the method of the present invention identifies a compound that is capable of inducing osteoblast-type cell differentiation and suppressing adipocyte-type cell differentiation. Preferably, the compound promotes or enhances the differentiation of osteoblast-type cells at the expense of adipocyte-type cells by producing a development switch in a common precursor of these cell types.

In one embodiment, the method is performed in a MSC. Methods of isolating MSCs are known in the art and described, for example, in Alejandro et al., Exp. Cell Res. 280: 24-32, 2002, Zuk et al, Mol. Biol. Cell, 13: 4279-4295, 2002 and Wickham et al., Clin. Orthop. 412: 196-212, 2003 (which are all incorporated herein by reference).

In one embodiment, a MSC is isolated from a long bone of a subject by isolating bone marrow cells from the bone and separating mononuclear cells using a Ficoll-Hypaque separation gradient and growing these cells in tissue culture for a time and under conditions to permit cells to adhere to the surface of a tissue culture vessel. Cells that do not adhere to the surface are removed and the remaining cells are passaged at least once. Those cells that remain adherent to the vessel are considered to be MSCs.

Alternatively, a MSC is isolated from adipose tissue, such as, for example a fat pad. Methods of isolating an MSC from adipose tissue are described, for example in, Zuk et al, Tissue Eng. 7: 211-226, 2001 and Zuk et al, Mol. Biol. Cell, 13: 4279-4295, 2002 (incorporated herein by reference). Alternatively, a MSC is isolated from the infrapatellar fat pad of the knee essentially as described in Wickham et al., Clin. Orthop., 412: 196-212, 2003 (incorporated herein by reference).

Mesenchymal stem cells are characterized by the expression of a number of proteins that are associated with mesenchymal precursors, such as, for example, SH2, SH3, SH4 and α-smooth muscle actin. Additionally these cells do not express the hematopoietic markers CD14m CD34 and CD45. Methods of detecting the expression of these markers of MSCs are known in the art and described, for example, in Alejandro et al, Exp. Cell Res., 280: 24-32, 2002.

In one embodiment, a MSC is derived from a human.

In a preferred embodiment, a MSC is derived from an animal subject. Even more preferably an animal subject that has a wild-type bone remodeling/bone growth phenotype. Preferably, a MSC is derived from a wild-type mouse. Even more preferably, a MSC is derived from a Y4^{-/-} mouse.

In one embodiment, a compound that is capable of modulating the differentiation of a MSC cell into an osteoblast type cell and/or an adipocyte type cell is screened to determine the effect of that compound on Y receptor activity and/or expression. Methods of determining Y receptor activity and/or expression are known in the art and/or described herein. Preferably the compound is capable of modulating Y receptor associated differentiation of a MSC cell.

In another embodiment, the method of the present invention comprises determining a compound that modulates Y receptor associated differentiation of a MSC cell and is incapable of crossing the blood brain barrier or crosses the blood brain barrier inefficiently or at a level that is inefficient to permit one or more undesirable Y receptor-mediated effects. Accordingly, the method of the present invention comprises the additional step of determining the ability of a compound to cross the blood brain barrier. Preferably, the compound is capable of modulating Y receptor associated differentiation of a MSC cell without crossing the blood brain barrier (ie without entering the brain). Methods of determining a compound that is not capable of crossing the blood brain barrier are known to those skilled in the art and/or described herein.

In a still further embodiment of the present invention, the method comprises the additional step of determining a compound that specifically modulates Y receptor associated differentiation of a MSC cell. By “specifically modulates Y receptor associated differentiation of a MSC cell” is meant that the compound is capable of modulating the differentiation of a MSC cell into an osteoblast type cell and/or an adipocyte type cell and the phenotypes associated with modulation of MSC differentiation in a subject (such as, for example, bone formation), but that the compound is not capable of modulating unassociated phenotypes (such as, for example, feeding behavior). It is preferred that the compound is not capable of modulating physiological processes that are associated with a Y receptor, excluding Y receptor associated differentiation of a MSC cell and phenotypes associated therewith. Methods of determining a compound that is capable of modulating Y receptor associated phenotypes are known in the art and/or described herein.

One embodiment of the present invention provides a method for determining a modulator of neuropeptide Y receptor associated MSC differentiation comprising administering a modulator of Y receptor activity to an animal subject and determining a the level of MSC differentiation into an osteoblast type cell and/or an adipocyte type cell, wherein a modified level of osteoblast type cell and/or an adipocyte type cell differentiation in the presence of the compound compared to the level of osteoblast type cell or an adipocyte type cell differentiation in the absence of the compound indicates that the compound is a modulator of Y receptor associated MSC differentiation.

Modulators of Y receptor activity are known in the art and described herein. Alternatively, a modulator of Y receptor activity is identified or produced using a method described herein.

A still further embodiment of the present invention provides a method for determining a modulator of neuropeptide Y receptor associated MSC differentiation comprising administering a modulator of MSC differentiation to an animal subject and determining a change in Y receptor activity, wherein a modified Y receptor activity in the presence of the compound compared to the Y receptor activity in the absence of the compound indicates that the compound is a modulator of Y receptor associated MSC differentiation.

Modulators of MSC differentiation are known in the art and include, for example BMP-2 and interleukin-6.

Methods of Treatment

The compounds identified using the screens described herein above for modulating a Y receptor-associated bone phenotype (i.e. Y receptor associated bone remodeling, Y receptor associated bone growth or Y receptor-associated adiposity) are useful for the treatment of any disorder associated with an aberrant Y receptor associated bone phenotype.

Moreover, the phenotype of reducing Y receptor expression and/or activity is more pronounced in a male subject, suggesting that modulatory compounds identified using the screening assays described herein are particularly useful in the treatment of one or more diseases or disorders associated with aberrant bone remodeling, aberrant bone formation, or aberrant adiposity in a male subject.

1. Treatment of a Bone Disorder

As will be apparent to a skilled artisan, a compound that is capable of modulating Y receptor associated bone remodeling (and in particular bone formation) is of particular use in the treatment of a bone disorder.

For example, a compound that is capable of inducing Y receptor associated bone remodeling is of particular use of treatment of bone disorders such as, for example osteomalacia, hyperostosis and osteoporosis, including involutional osteoporosis, post-menopausal osteoporosis, senile osteoporosis and steroid (glucocorticoid osteoporosis), treatment of bone segmental defects, periodontal defects, metastatic bone disease, and osteolytic bone disease (such as, for example, myeloma).

Alternatively, a compound that is capable of suppressing Y receptor associated bone remodeling is of particular use in the treatment of a disease such as, for example, osteopetrosis.

The treatment of males or females, and particularly male subjects, is clearly encompassed by the present invention.

In one embodiment, the present invention provides a method of treatment of a disorder associated with bone remodeling and/or bone formation comprising administration of an amount of a compound sufficient to modulate Y receptor bone remodeling in a cell.

In one embodiment, the present invention provides a method of treatment of a bone disease comprises administering an amount of a compound identified using a screening method of the present invention in an amount effective to modulate Y receptor bone remodeling or bone formation. An effective amount is an amount sufficient to achieve the desired therapeutic or prophylactic effect, under the conditions of administration, such as an amount sufficient for inhibition or promotion of Y receptor associated bone remodeling.

In a preferred embodiment, a method of treatment comprises modulating the expression of a Y receptor such that Y receptor associated bone remodeling is modulated. In a particularly preferred embodiment, the expression of a Y2 receptor is modulated in the hypothalamus of a subject to induce bone formation in that subject.

Methods of modulating expression of a gene product are known in the art and include, for example, the administration of an antisense nucleic acid, ribozyme, PNA, or interfering RNA.

In another embodiment, a method of treatment comprises administration of a compound that is capable of modulating the activity of a Y receptor and thereby modulating Y receptor associated bone remodeling. Preferably, the compound is capable of suppressing Y receptor activity, thereby inducing the formation of new bone. As will be apparent to the skilled artisan, numerous means exist for modulating the activity of a protein, such as, for example, inhibiting the binding of a receptor to a ligand or inhibiting the signaling of a receptor following binding of a ligand.

In one embodiment, a method of treatment comprises modulating Y receptor activity and determining a change in a bone phenotype. Methods of determining a change in bone phenotype are known in the art and/or described herein.

In a related embodiment, a method of treatment comprises administration of an amount of a compound sufficient to modulate Y receptor activity and determining a change in a bone phenotype.

In another embodiment, the method of the previous embodiment comprises the additional step of determining a change in bone density. Methods of determining bone density in a subject are known in the art, and include, for example, dual X-ray absorptiometry on the lumbar spine, femoral neck, radius and calcaneus; peripheral quantitative computed tomography (PQCT) on the radius; and quantitative ultrasound (QUS) on the calcaneus (essentially as described in Ito et al., Osteoporisis Int., 29, 2003 and Fang et al., J. Clin. Densitom. 5(4): 421-433, 2002).

A further embodiment of the present invention provides a method of treatment of a subject comprising isolation of a mesenchymal stem cell (MSC) from the subject, treatment of the MSC with a compound that modulates Y receptor associated MSC differentiation in order to induce differentiation of the MSC into an osteoblast type cell and introducing the osteoblast type cell into the subject.

Accordingly, this embodiment provides an ex vivo method for the induction of osteoblast development. As demonstrated in U.S. Pat. No. 6,541,024 introduction of a MSC that has been induced to differentiate into an osteoblast type cell into a subject effectively induces bone formation in a femoral gap model.

In an alternative embodiment, the present invention provides a method of treatment of a subject comprising isolating a MSC from the subject, silencing at least one Y receptor gene in the cell in order to induce differentiation of the MSC into an osteoblast type cell and introducing the osteoblast type cell into the subject.

In one embodiment, at least one Y receptor is silenced using a compound such as, for example an antisense nucleic acid, ribozyme, PNA, or interfering RNA.

In another embodiment, at least one Y receptor is silenced using homologous recombination using methods known in the art and/or described herein.

In one embodiment, the method of the present invention incorporates the additional step of expanding or growing the MSC cells or osteoblast type cells in order to produce numbers sufficient for the treatment of a bone disorder.

In one embodiment, differentiated osteoblast type cells are introduced directly into the bone of a subject using methods known in the art, such as, for example, surgery.

Alternatively, when treating a localized bone disorder, such as, for example, a fracture, it is preferred that a differentiated osteoblast cell is introduced at the site of the disorder. The efficacy of such a method of treatment is demonstrated in U.S. Pat. No. 6,541,024. More preferably, a differentiated osteoblast type cell is introduced into a subject by infusion into the blood stream, from where the osteoblast cell is recruited to a bone of the subject.

A still further embodiment of the present invention provides a method of modulating the formation of bone in vitro, comprising incubating a sample of bone, or cells isolated from bone in an amount of a compound that modulates Y receptor associated bone remodeling sufficient to produce new bone.

In an alternative embodiment, a method of modulating the formation of new bone in vitro comprises silencing the expression of at least one Y receptor in a sample of bone or cells isolated from bone for a time sufficient to induce the formation of new bone. Methods of silencing the expression of a Y receptor will be apparent to the skilled artisan and include the use of an antisense nucleic acid, ribozyme, PNA, or interfering RNA.

A related embodiment of the present invention provides a method of treatment of a subject comprising incubating a sample of bone, or cells isolated from bone in an amount of a compound that modulates Y receptor associated bone remodeling sufficient to produce new bone and administration of the new bone to the subject.

Preferably, the new bone is administered to a site at which the new bone is required, such as, for example, at the site of a fracture.

A still further embodiment of the present invention provides the use of a modulator of Y receptor bone remodeling in the manufacture of a medicament for the treatment of a bone disorder. Preferably, the bone disorder is selected from the group consisting of osteomalacia, hyperostosis and osteoporosis, including involutional osteoporosis, post-menopausal osteoporosis, senile osteoporosis and steroid (glucocorticoid osteoporosis), treatment of bone segmental defects, periodontal defects, metastatic bone disease, and osteolytic bone disease (such as, for example, myeloma).

A further embodiment of the present invention provides the use of an a MSC in which at least one Y receptor has been silenced in the manufacture of a medicament for the treatment of a bone disorder. Preferably, the bone disorder is selected from the group consisting of osteomalacia, hyperostosis and osteoporosis, including involutional osteoporosis, post-menopausal osteoporosis, senile osteoporosis and steroid (glucocorticoid osteoporosis), treatment of bone segmental defects, periodontal defects, metastatic bone disease, and osteolytic bone disease (such as, for example, myeloma).

2. Treatment of Obesity

As will be apparent to the skilled artisan a compound that is capable of suppressing the differentiation of a MSC cell into an adipocyte is of particular use in the treatment of obesity. The suppression of the formation of adipocytes will clearly result in a reduction in the amount of fat in a subject.

As exemplified herein, suppression of expression of at least one Y receptor reduces the total body fat content of an animal subject. In particular, the suppression of the expression of a Y2 receptor reduces the total body fat content, in addition to the amount of white adipose tissue in an animal subject. Interestingly, when crossed onto the ob/ob (leptin deficient) mouse background, Y2^{-/-}ob/ob mice the increased adiposity of the ob/ob mice is suppressed. Furthermore suppression of the expression of Y2 and Y4 receptors caused a significant reduction in the bodyweight of the animal subject.

Accordingly, the method of the present invention provides a method of treating an obese subject comprising administering an amount of a compound that modulates Y receptor mediated adiposity sufficient to modulate Y receptor mediated adiposity. Preferably, the compound suppresses Y receptor mediated adiposity.

In one embodiment, the compound is one that modulates the differentiation of a MSC to an adiposite. A preferred compound suppresses the differentiation of a MSC to an adiposite.

In one embodiment, a compound that modulates Y receptor mediated adiposity comprises nucleic acid. Preferably, the nucleic acid is an antagonist of expression of at least one Y receptor, such as, for example, an antisense nucleic acid, PNA, ribozyme, or interfering RNA.

In accordance with this embodiment, a method of treatment of a subject comprises administration of an amount of a compound that suppresses expression of at least one Y receptor sufficient to modulate Y receptor mediated adiposity.

The use of antibodies or ligands that can inhibit Y receptor mediated adiposity, such as a binding activity, a signalling activity is also encompassed by the present invention. In one embodiment, antibodies or ligands of the present invention can inhibit binding of a ligand (i.e., one or more ligands) to a Y receptor and/or can inhibit one or more functions mediated by a Y receptor in response to ligand binding. In a particularly preferred embodiment, the antibody or ligand can inhibit (reduce or prevent) the interaction of a Y receptor with a natural ligand such as neuropeptide Y.

A further embodiment of the present invention provides the use of a modulator of Y receptor associate adiposity in the manufacture of a medicament for the treatment of obesity.

### EXAMPLE 1

### Targeted Disruption of the Neuropeptide Y2 Receptor Results in Increased Bone Volume, Increased Trabecular Number and Increased Trabecular Volume

Germline Y2 receptor deficient mice were generated using homologous recombination in embryonic stem cells essentially as described in Baldock et al. J. Clin Invest., 109(7): 915-921, 2002. Essentially, mouse ES cells (129SvJ) were transfected with a targeting construct designed to flank the coding region of the Y2 receptor gene, which is incorporated in a single exon, with loxP sites. Cells that had successfully incorporated the targeting construct were selected using standard methods and positive cells were injected into blastocysts isolated from C57/BL6 mice. Chimeric offspring were mated with C57/BL6 mice expressing Cre recombinase under the control of an oocyte specific promoter (these mice are produced using methods described in Schwenk et al., Nucleic Acids Res. 23: 5080-5081, 1995, incorporated herein by reference). This cross produced heterozygote mice carrying the Y2 receptor gene flanked by loxP sites (ie floxed) (Y2^{lox/+}) and heterozygote mice expressing Cre recombinase in which the Y2 receptor gene was deleted (Y2^{-/-}). Homozygous mice were then generated (ie Y2^{lox/lox} and Y2^{-/-}) by crossing the respective homozygous animals.

Skeletal phenotypes of Y2^{-/-} mice were then analyzed in the femora. Femora were excised and bisected transversely at the midpoint of the shaft. The distal parts of the right femora were fixed and embedded, undecalcified, in K-plast resin (Medium-Medizinisded Diagnostik, Giessen, Germany), and 5 μm sagittal sections were prepared for analysis using BioQuant software (R&M Biometrics Inc., Nashville Tenn., USA).

Sections were also stained for mineralized bone according to Page (In: Theory and Practice of Histological Techniques, Bancroft and Stevens eds. Churchill Livingstone, London, 1977), and trabecular bone volume, thickness and number were calculated using the methods of Parfitt et al. J. Clin. Invest. 72: 1396-1409, 1983. Osteoblast surface, osteoblast number and osteoid surface were estimated using sections stained with von Kossa stain and toluidine blue.

Bone formation (mineralizing surface) was estimated using the bone surface coverage of single- and double-labeled fluorescent bands. Mice were injected with the fluorescent tetracycline compounds calcein and demeclocycline (15 mg/kg each; Sigma Chemical Co., St. Louis, Mo., USA) days and 3 days prior to tissue collection, respectively. Bone formation (mineralizing surface, MS) was estimated using the bone surface (BS) coverage of single-labeled (sLS) and double-labeled (dLS) fluorescent bands using the equation MS={[(0.5×sLS)+dLS]×100}/BS, expressed as a percentage of BS. Mineral apposition rate (MAR) was estimated by the distance between the labels divided by the time interval between injection of labels (MAR=inter-label distance divided by 7, expressed in μm/d). Bone formation rate (BFR) was calculated after fluorescence microscopy (Leica Microsystems, Heerbrugg, Switzerland) as BFR=MS/BS×MAR, expressed in μm²/μm/d) (as described by, Parfitt et al., J. Bone Miner. Res., 2: 595-610, 1987).

In order to determine osteoclast surface and osteoclast number sections were stained for tartrate-resistant acid phosphatase (IRAP) activity as described by Hayman et al., J. Histochem. Cytochem., 49: 675-684, 2001.

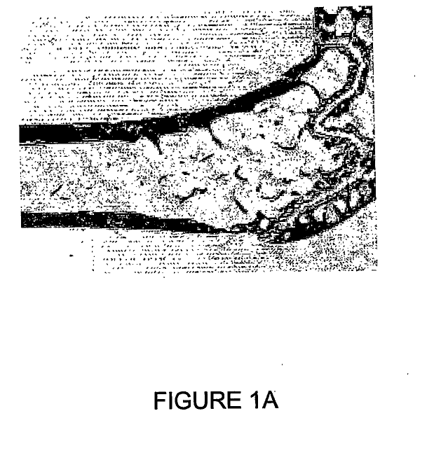

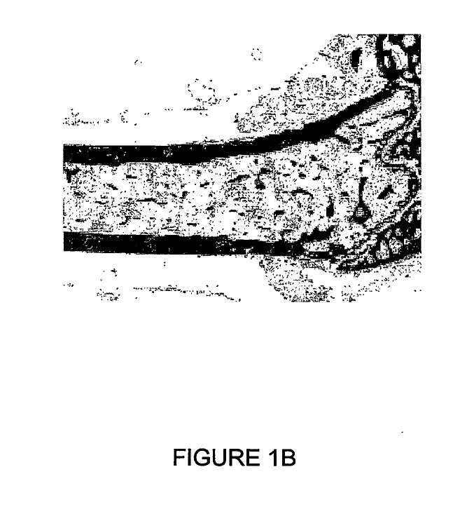

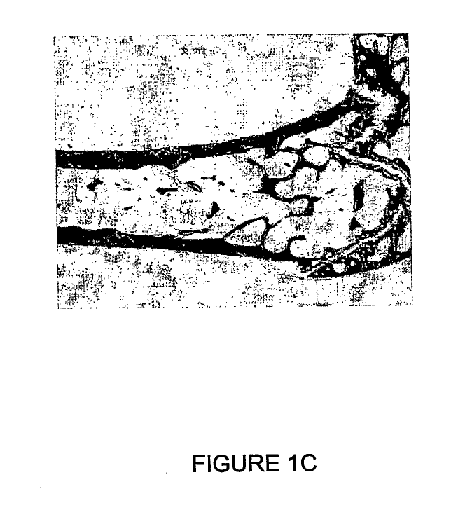

By 16 weeks of age Y2^{-/-} mice showed a dramatic increase in both trabecular abundance and in trabecular structure as shown in FIGS. 1A to 1C ((wt) (A), Y2^{-/+}; (B); and Y2^{-/-} (C)). Furthermore, this effect appeared to be dosage dependent with Y2^{+/-} (FIG. 1B) mice showing a thickening of the trabecular bone compared to wt (FIG. 1A), while there was an increase in trabecular number and thickness in Y2^{-/-} mice (FIG. 1C) compared to wt and heterozygous mice.

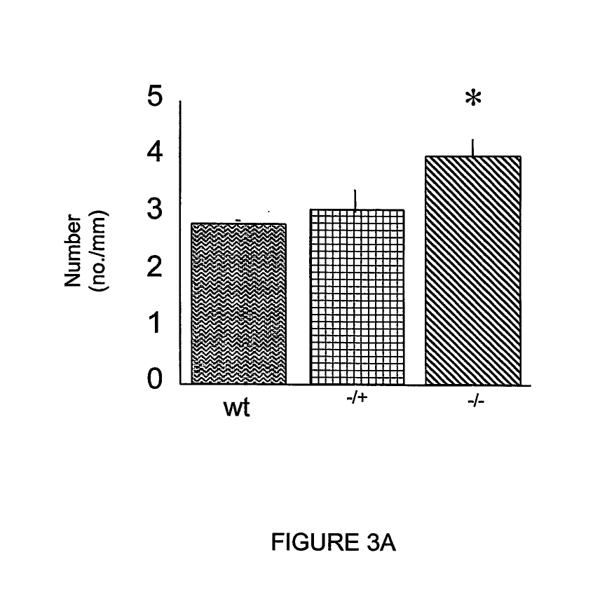

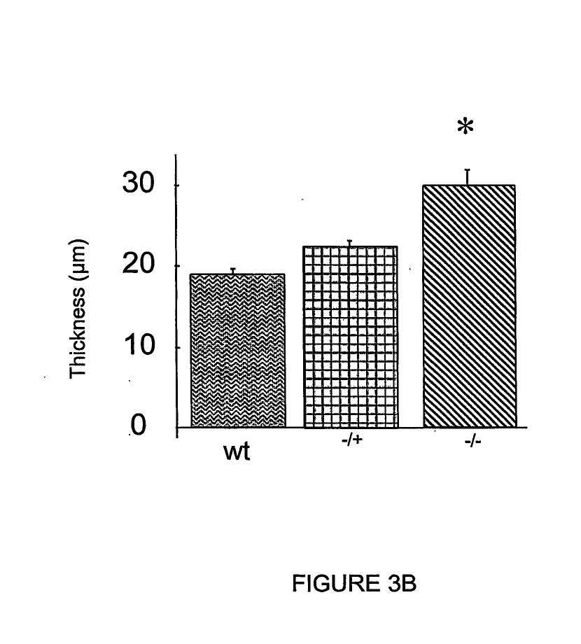

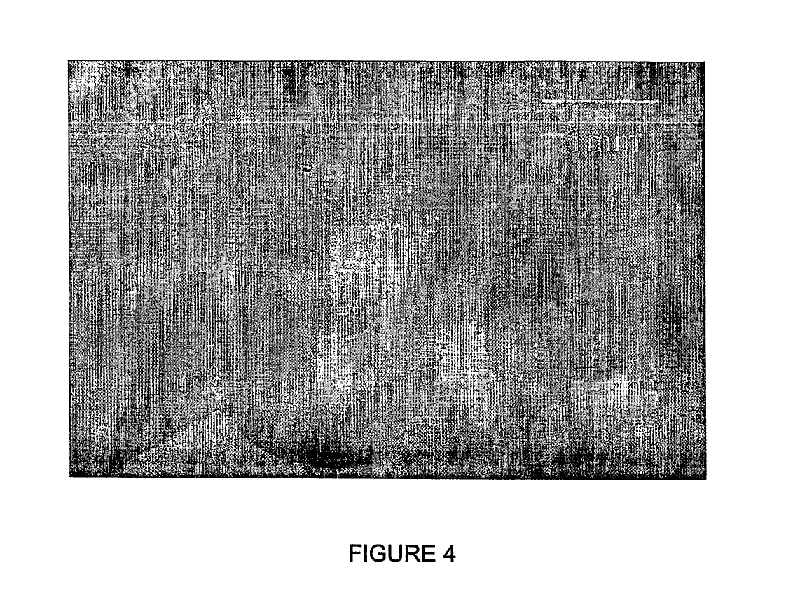

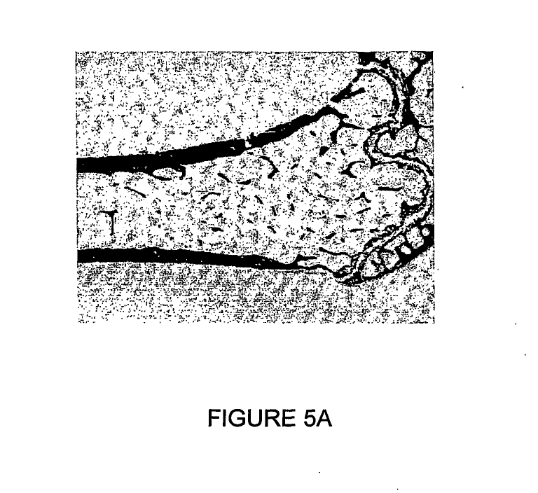

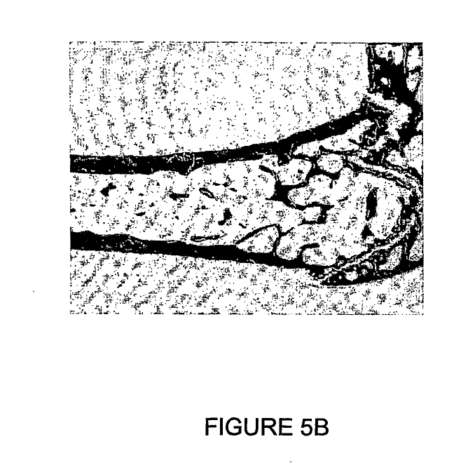

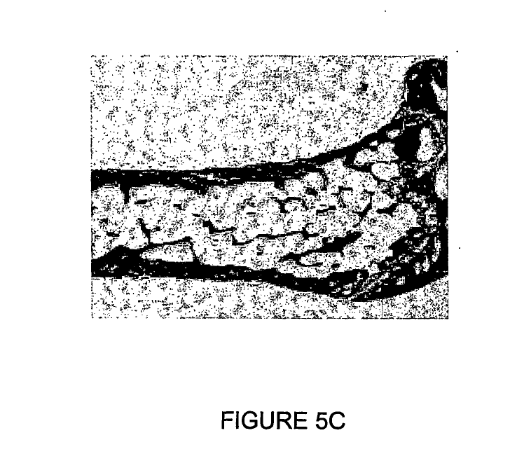

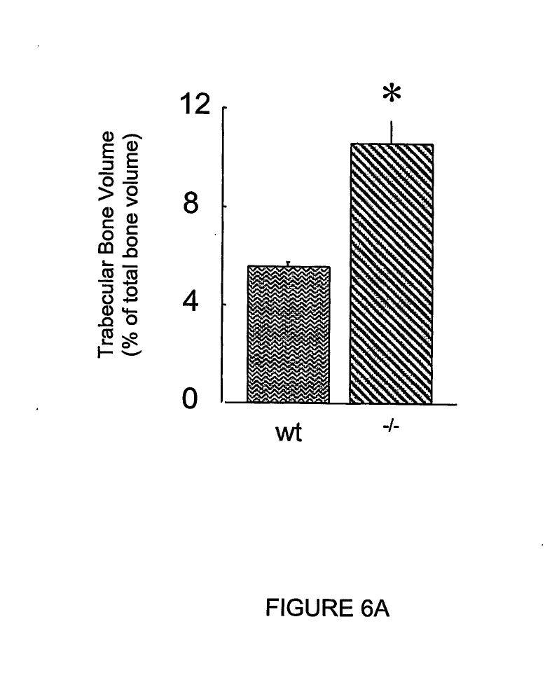

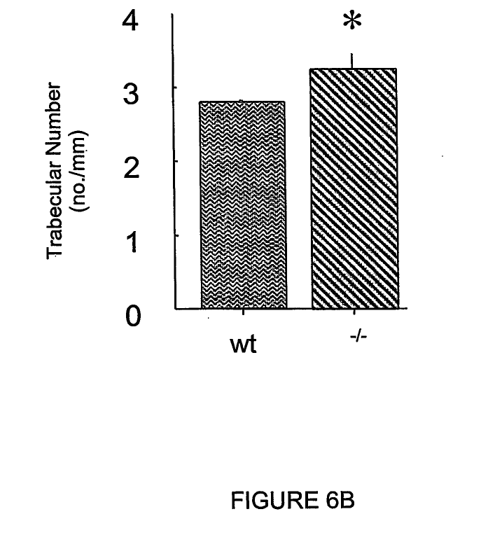

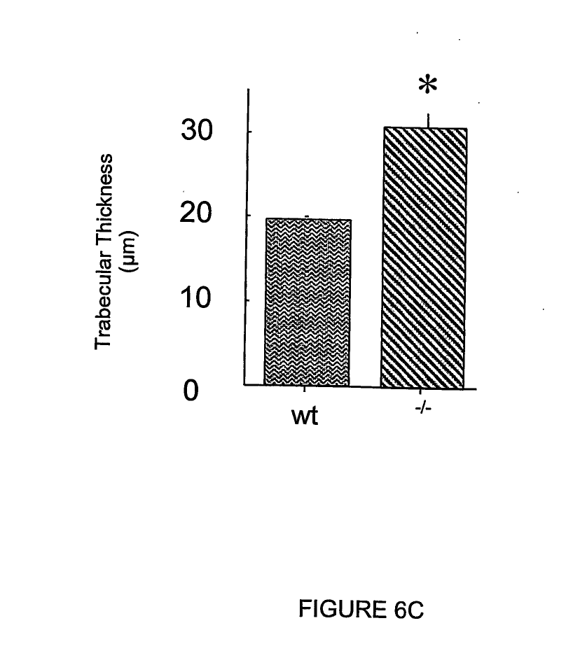

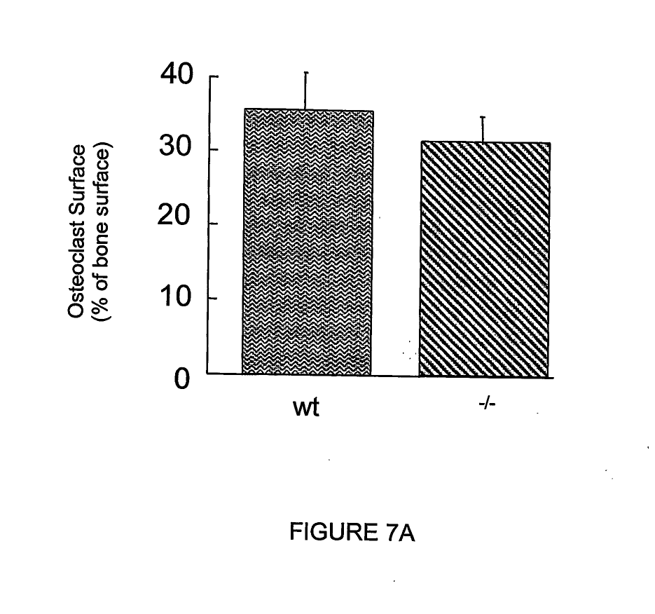

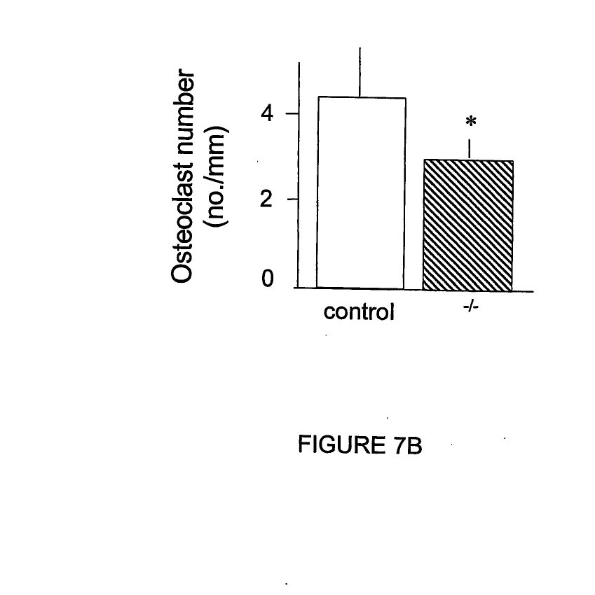

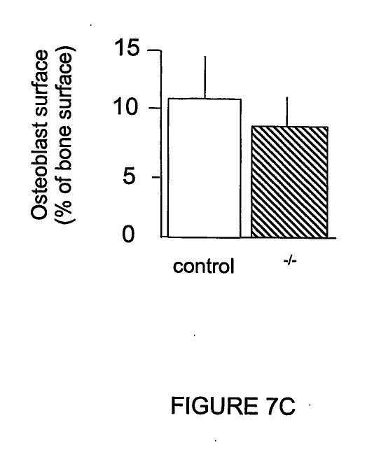

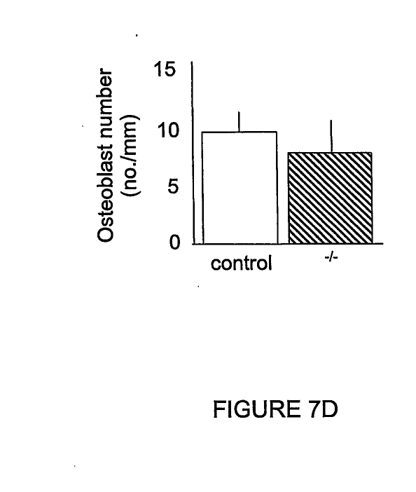

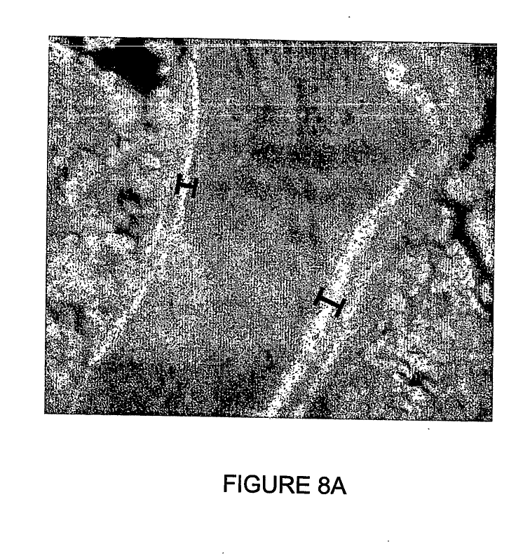

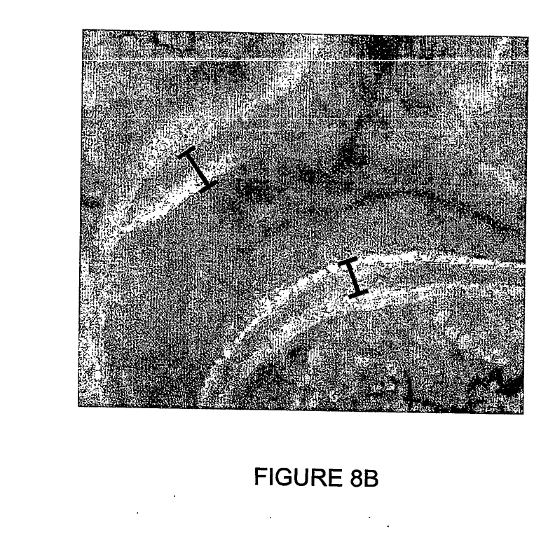

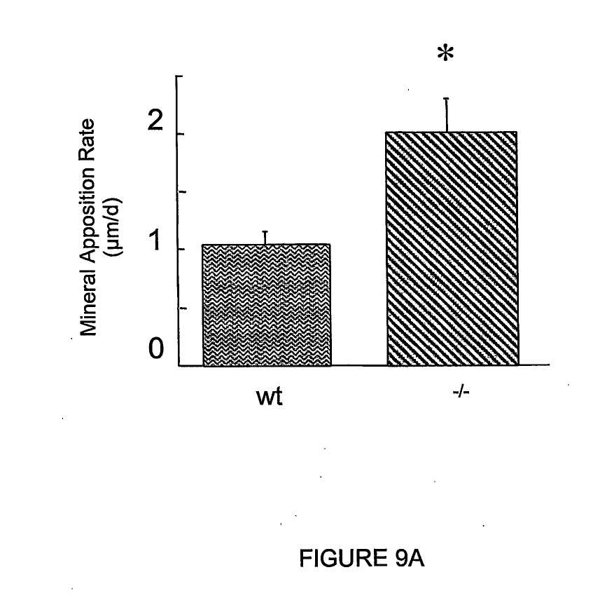

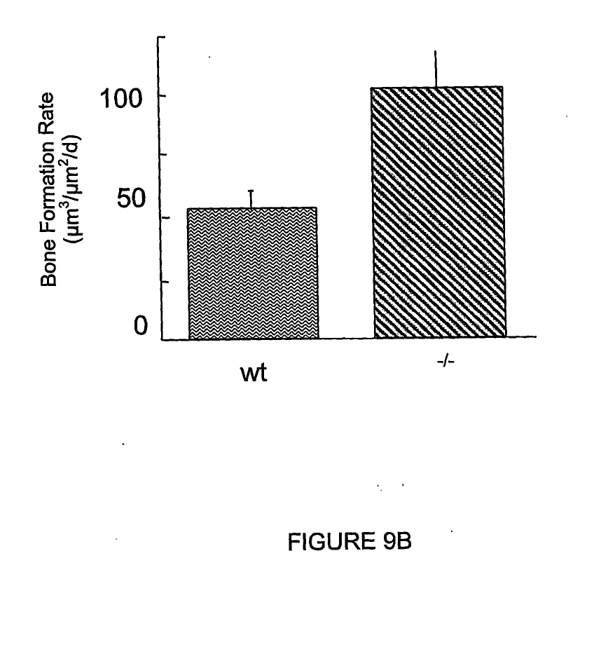

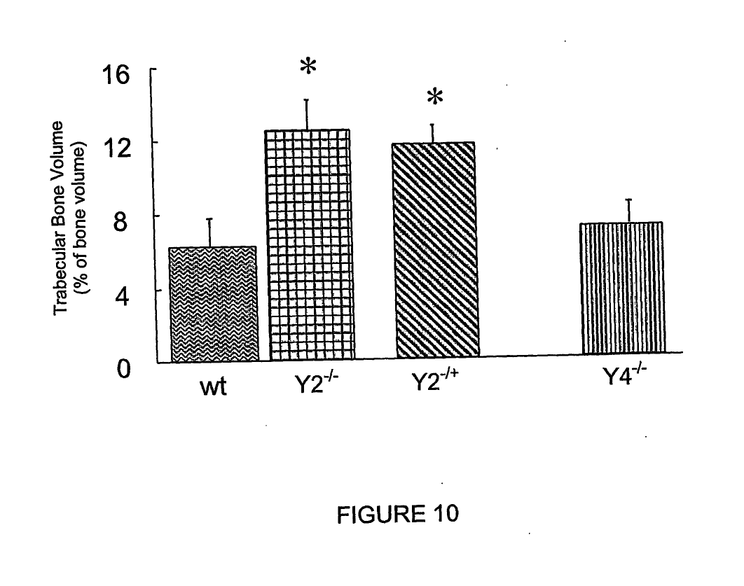

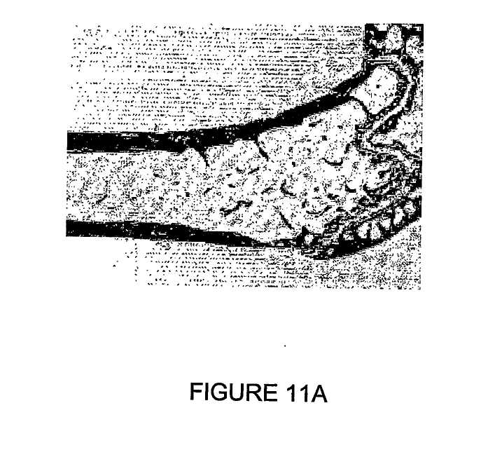

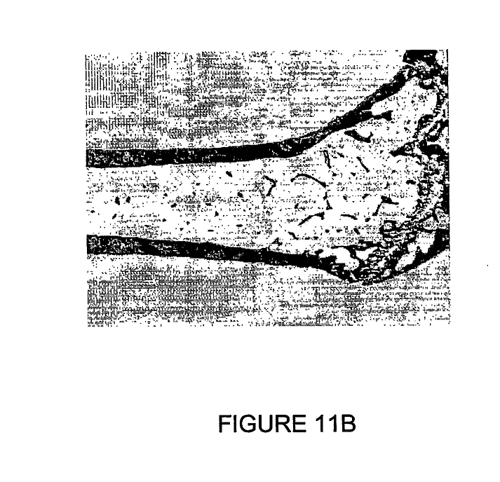

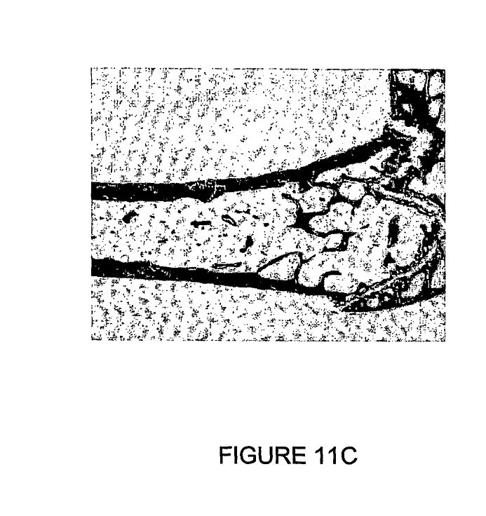

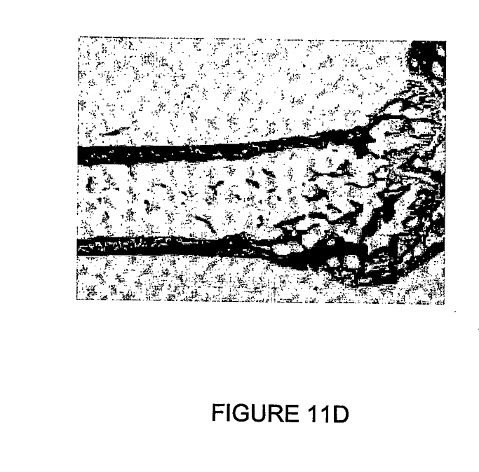

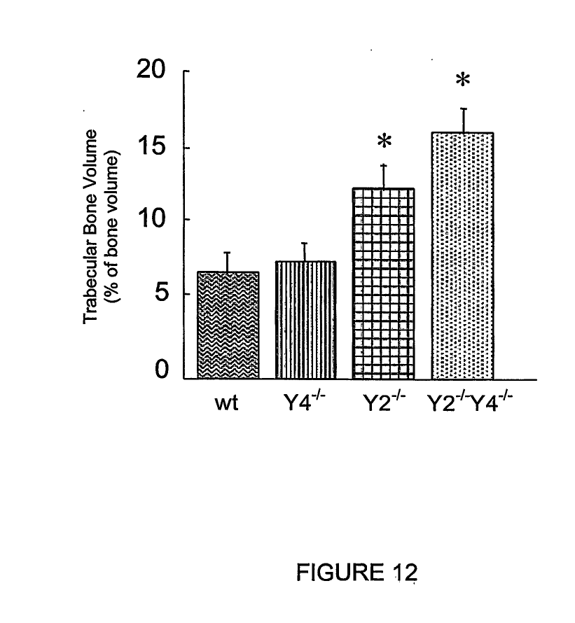

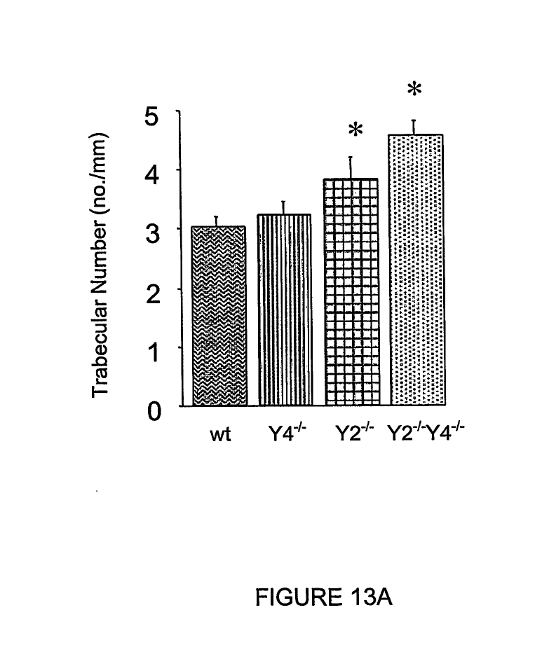

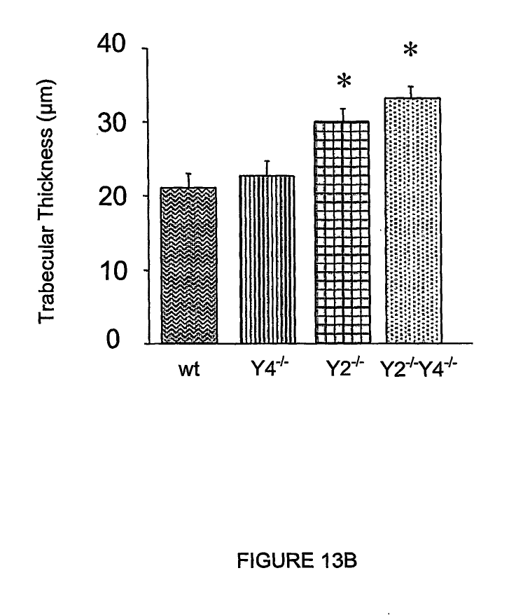

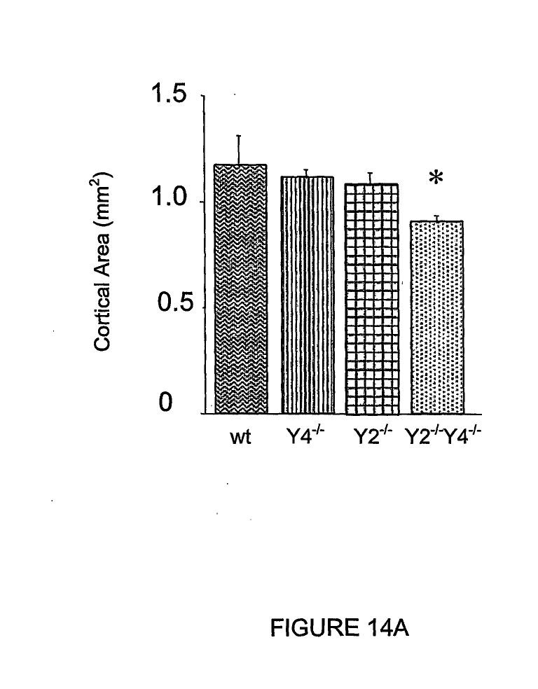

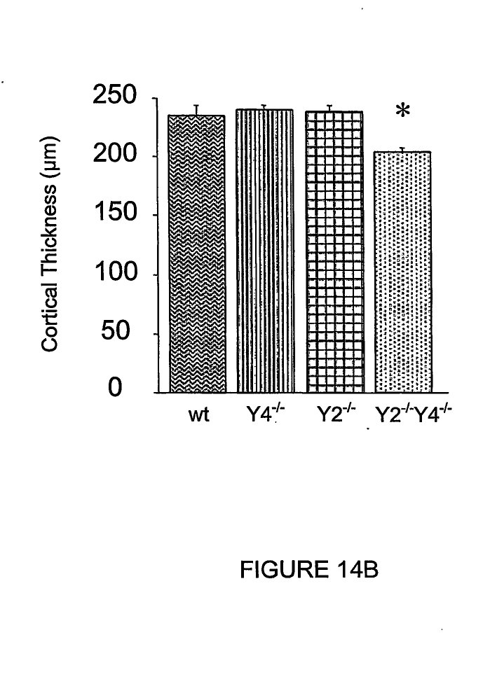

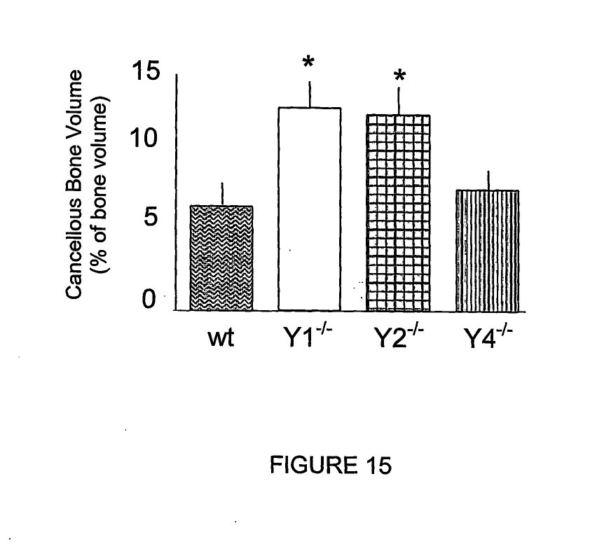

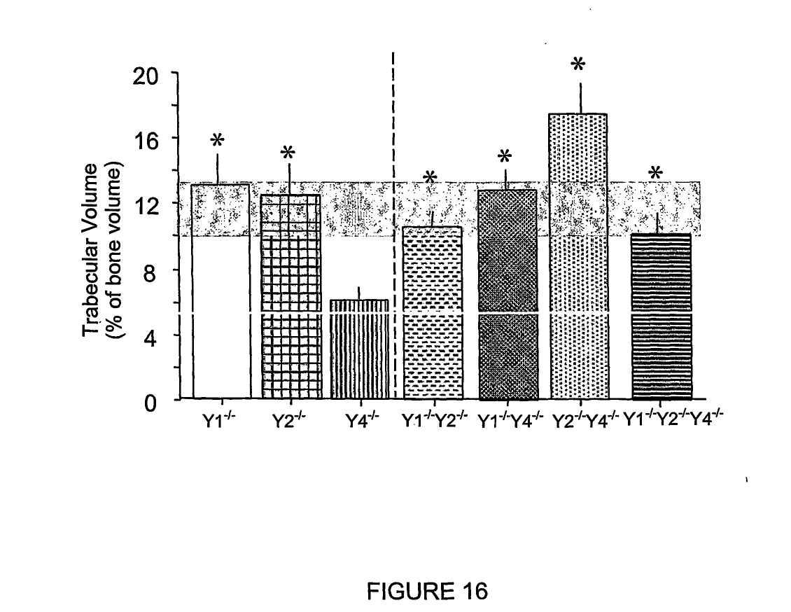

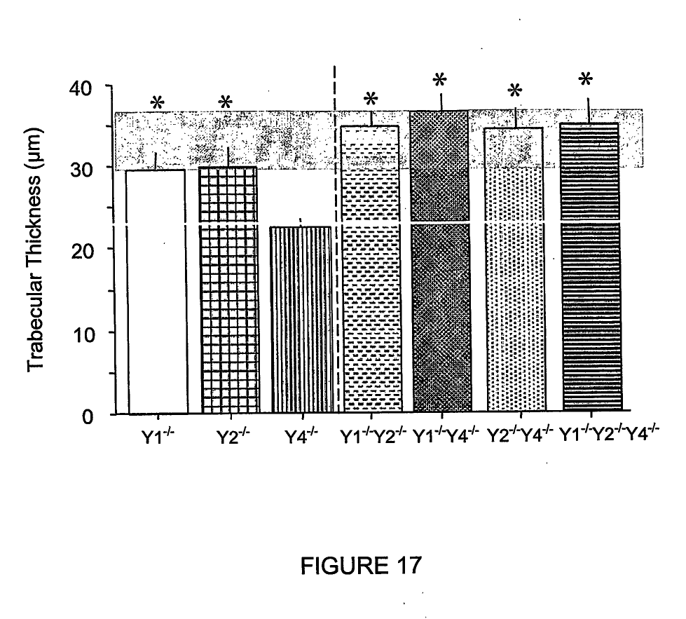

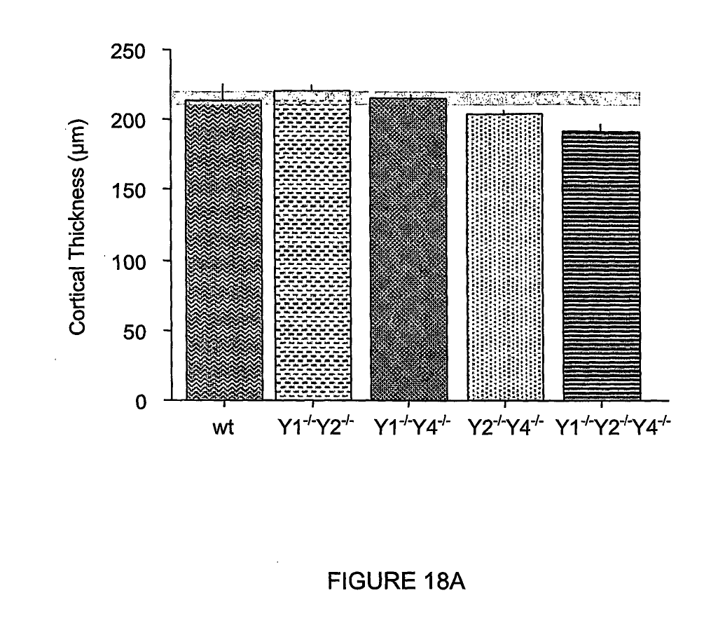

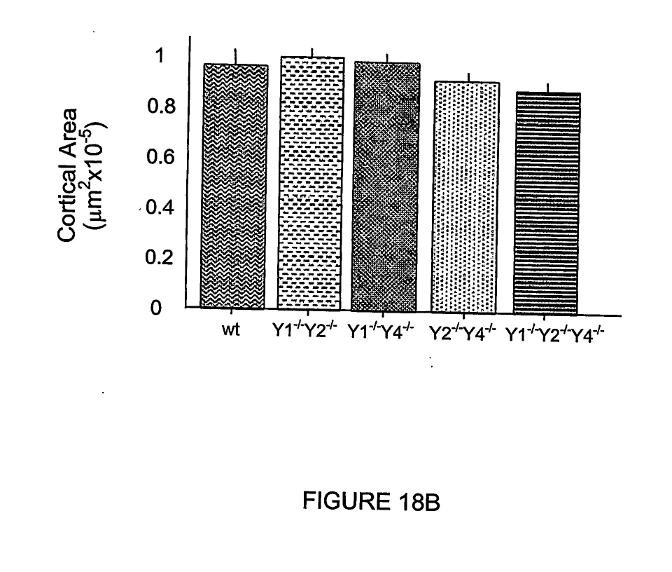

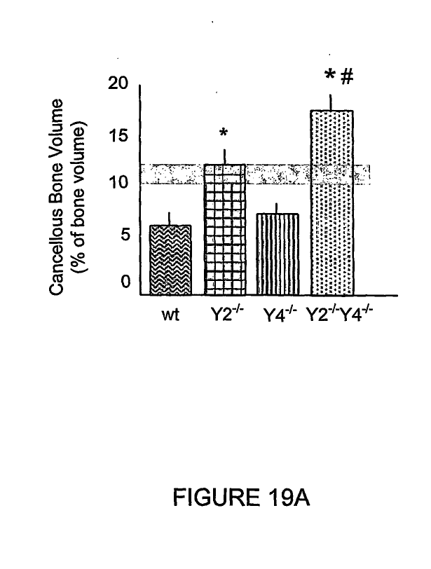

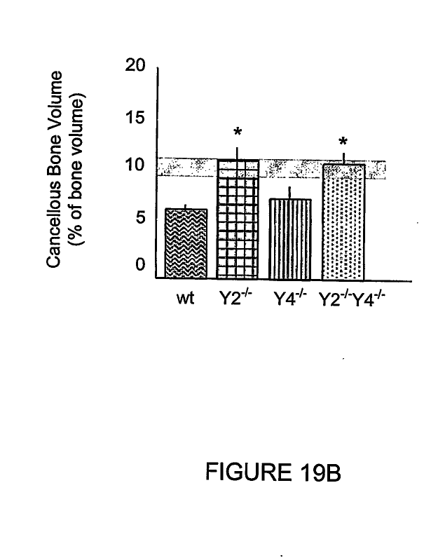

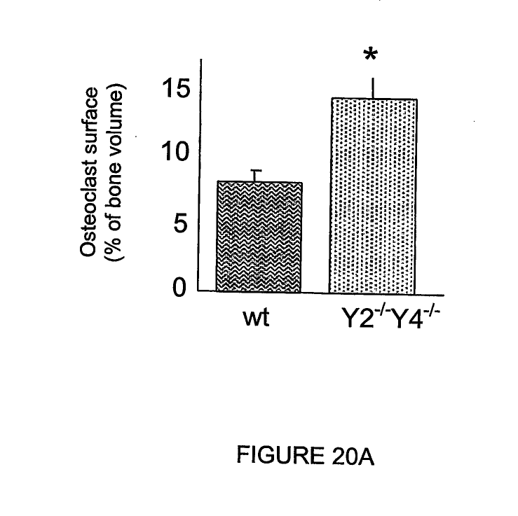

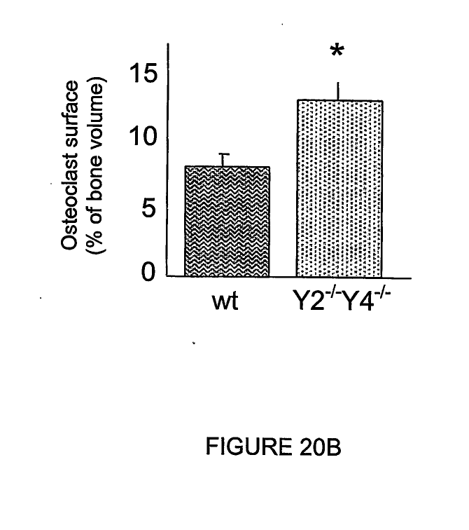

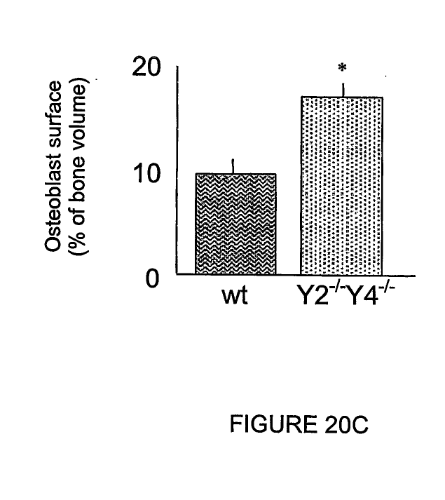

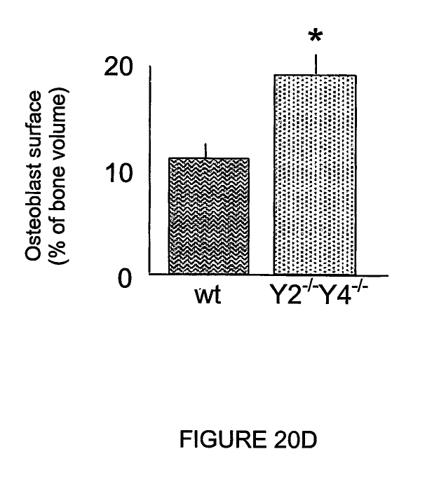

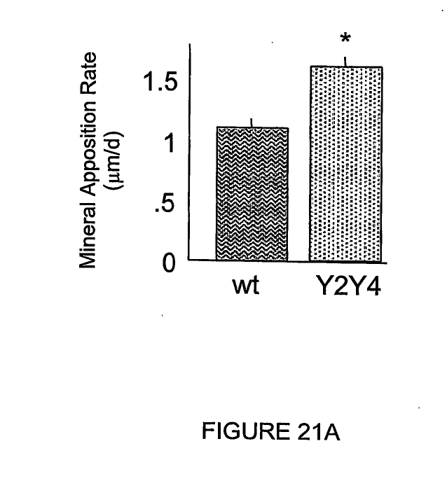

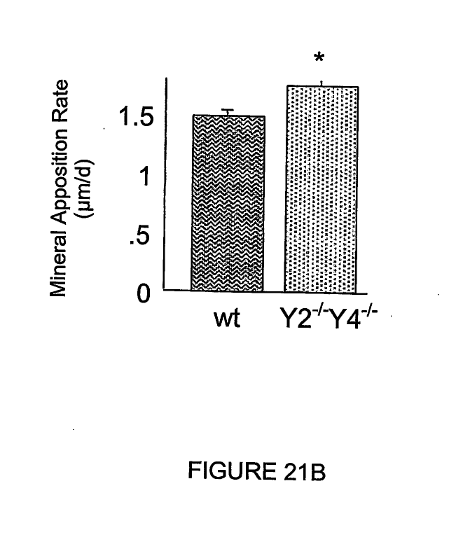

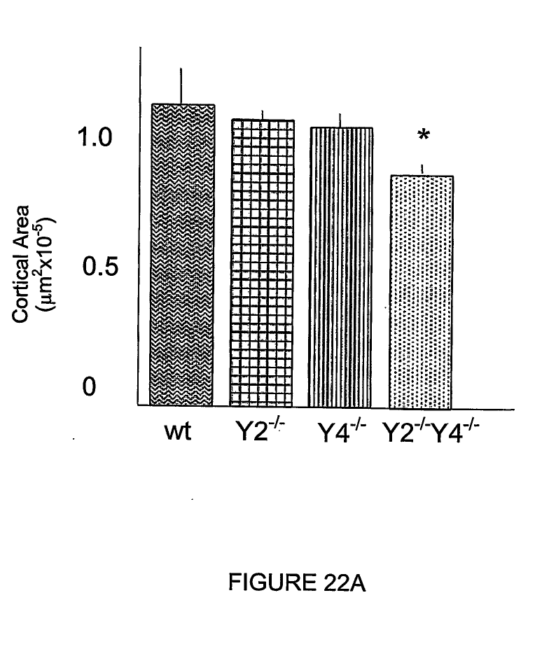

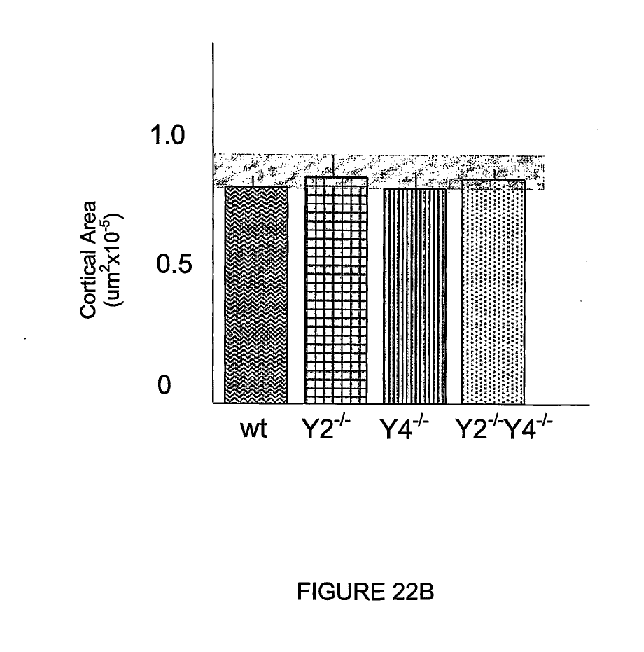

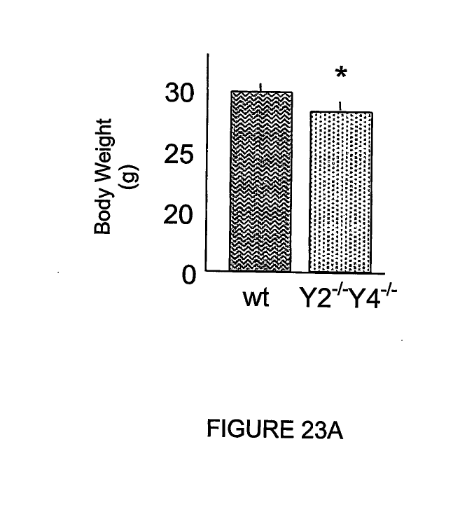

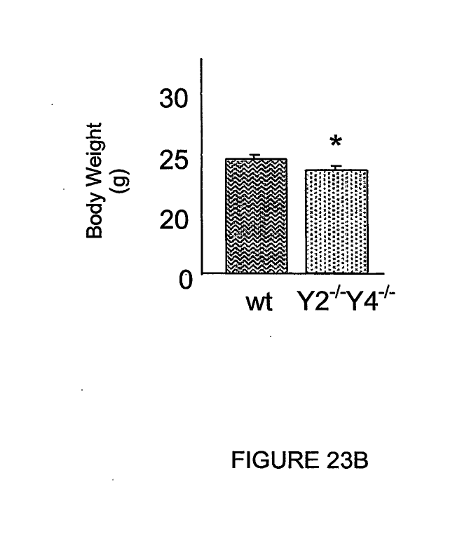

As shown in FIG. 2 trabecular bone volume was significantly increased in Y2^{-/-} mice compared to wt and Y2^{+/-} mice. This increased trabecular volume of Y2^{-/-} animals was associated with a significant increase in both trabecular number (FIG. 3a) and trabecular thickness (FIG. 3b).

The increase in bone volume in Y2^{-/-} mice as a result of increased trabecular volume and thickness adds to the overall strength of the bone in these mice.

In order to determine the site of activity of neuropeptide Y in the formation of bones RT-PCR was used to determine which, if any, of the Y receptors were expressed in bone tissue. Results of this analysis suggested that none of the known Y receptors were expressed in bone tissue. Accordingly the site of action of neuropeptide Y on bone remodeling must be at a site other than the bone itself.

### EXAMPLE 2

### Tissue Specific Targeted Disruption of the Neuropeptide Y2 Receptor Results in Increased Bone Volume, Increased Trabecular Number and Increased Trabecular Volume

In order to study the effect of targeted disruption of the neuropeptide receptor Y2 receptor in the hypothalamus Y2^{lox/lox} mice (described in Example 1) and Y2^{+/+} mice were injected with an adenovirus that expressed Cre recombinase or green fluorescent protein (GFP) (The Institute of Physical and Chemical Research, Riken, Fukyuku, Japan). Approximately 10⁹ plaque forming units of either virus were bilaterally injected into the arcuate nucleus. Bones were isolated from these mice and analyzed 35 days later when mice were 15-17 weeks of age using the methods described in Example 1.

In these experiments, the two groups of control mice (GFP-injected Y2^{lox/lox} and Cre-injected Y2^{+/+} mice) were indistinguishable from each other for all parameters investigated.

Appropriate positioning of the injection was monitored by the appearance of green fluorescence in the hypothalamus of GFP-injected animals (FIG. 4). Cre gene expression and consequent Y2 receptor gene deletion in the hypothalamus were confirmed at 35 days after adenovirus injection by in situ PCR analysis of coronal brain sections isolated from these animals (data not shown).

The selective deletion of hypothalamic Y2 receptors in adult mice resulted in a bone phenotype analogous to that observed in the germline Y2^{-/-} mice described in Example 1. As shown in FIGS. 5A to 5C (wt (A), GFP-Y2^{lox/lox} (B) and Cre-Y2^{lox/lox} (C)) mice injected with a Cre recombinase expressing adenovirus showed a similar phenotype in the trabecular bone volume of the distal femoral metaphysis, with increases in both trabecular number and thickness. A twofold increase in trabecular bone volume was produced during the 5-week period of hypothalamic Y2 receptor deficiency (FIG. 6a).

Consistent with germline Y2 receptor knockout, hypothalamic Y2 deletion resulted in increased trabecular number and trabecular thickness (FIGS. 6b and 6c). There was no indication of change in resorption as measured by osteoclast surface (FIG. 7A), but osteoclast number was significantly reduced (FIG. 7B). Osteoblast surface (FIG. 7C), osteoblast number (FIG. 7D), osteoid surface, and mineralizing surface were all unaffected by hypothalamic Y2 receptor deletion. However, the rates of bone mineral apposition and bone formation were significantly increased by the hypothalamic Y2 receptor deletion (FIGS. 9A and 9B). This measure of the rate of bone mineralization, as indicated by the distance between the two fluorescent bands, indicates that hypothalamus-specific Y2 receptor knockout animals formed substantially more bone than control animals (FIGS. 8A and 813) (wt, (A); and Cre-Y2^{lox/lox} (13)). Indeed, bone mineral apposition rate and the associated bone formation rate in the conditional knockouts were both around twofold higher than these rates in controls (FIG. 9, a and b).

Of particular interest in this study was the observation that modulation of Y2 receptor expression was capable of inducing changes in bone remodeling in adult animals, suggesting that modulation of the Y receptors may be of important therapeutic benefit in the treatment of adult onset disorders, such as, for example, osteoporosis.

Results of the study of the Y2^{-/-} mice and the conditional Y2^{-/-} mice provides the first evidence that hypothalamic Y2 receptors inhibit bone formation. The absence of detectable levels of Y receptor mRNAs in bone tissue provides evidence that this effect of Y2 deficiency occurs by a central mechanism.

Trabecular bone volume and the rate of bone mineralization and formation are increased in Y2 knockout mice, with no increase in osteoblast or osteoid surface, or osteoblast number. Thus deletion of hypothalamic Y2 receptors acts to release a tonic inhibition of the activity of trabecular osteoblast activity, increasing the rate of bone mineralization and formation twofold.

Osteoclast surface was not affected by Y2 deletion, suggesting an increase in osteoclast size in these knockouts. Such a change in osteoclast morphology is consistent with an increase in resorptive activity per cell.

These data clearly indicate a major role of hypothalamic Y2 receptors in the regulation of bone mass. The rapid increase in bone volume in adult mice after central deletion of Y2 receptor suggests new possibilities for the prevention and anabolic treatment of osteoporosis.

### EXAMPLE 3

### Targeted Disruption of the Y4 Receptor Does Not Affect Bone Remodeling

Targeted disruption of the Y4 receptor was performed as described in Sainsbury et al., Genes and Development, 16: 1077-1088, 2002 (incorporated herein by reference). Essentially, a targeting vector for the Y4 receptor gene was designed which allowed the production of both germ-line (Y4^{-/-}) and conditional (Y4^{lox/lox}) knockout mice. A cassette containing the neomycin resistance gene (Neo) flanked on either side by a 34 bp-long Cre-recombinase recognition (loxP) site oriented in the same direction was placed downstream of the Y4 receptor gene. A third loxP sequence was inserted into intron I of the Y4 receptor gene. Mouse embryonic stem (ES) cells from the strain 129/SvJ were transfected and selected under standard conditions. Positively targeted clones were identified by Southern analysis.

Positive ES cell clones for the Y4 receptor construct were injected into blastocysts originating from C57BL/6 mice and implanted into pseudopregnant mice. Offspring with the highest percentage of agouti coat color were crossed with X chromosome-linked, oocyte-specific Cre-recombinase-expressing C57BL/6 mice (Schwenk et al. 1995) to obtain either heterozygotes carrying the floxed gene (conditional, Y4^{+/lox}), or heterozygotes carrying the Cre-recombinase gene and having the floxed gene already deleted (germ line Y4^{+/-}). Absence of the Y4 gene in Y4^{-/-} mice was confirmed by Southern analysis using a DNA fragment specific for the Y4 receptor coding sequence.

As shown in FIG. 10 targeted disruption had no apparent effect on the trabecular bone volume of the distal femoral metaphysis. In fact, when analyzed at the histological level the bones of Y4^{-/-} (FIG. 11a) mice appeared no different to wt (FIG. 11b) mice, with no apparent increase in the number of trabecular or thickness of the trabeculae.

Proceeding on this basis, the Y4 receptor appears to have no effect on bone remodeling.

### EXAMPLE 4

### Targeted Disruption of Y4 Receptor Increases the Sensitivity of Mice to Modulators of Bone Remodeling

Mice in which the Y4 receptor has been disrupted (described in Example 3) were crossed with Y2 deficient mice (described in Examples 1 and 2) to generate Y2^{-/-}Y^{-/-} mice. The genotype of these mice was confirmed by Southern blot analysis. The morphology of the bones of these mice were then analyzed using methods described in Example 1.

As shown in FIGS. 11A to D (11A, wild-type; (B) Y4^{-/-}; (C), Y2^{-/-}; (D) Y2^{-/-}Y4^{-/-}) Y2^{-/-}Y4^{-/-} mice exhibit a profound increase in bone volume, exceeding even that increase observed in the Y2^{-/-} mice. Trabecular bone volume was increased in Y2^{-/-}Y^{-/-} over that of wt and Y4^{-/-} mice (FIG. 12), as was trabecular number (FIG. 13a) and trabecular thickness (FIG. 13b). Moreover, Y2^{-/-}Y4^{-/-} mice showed even greater increases in these parameters over control mice than the observed in the Y2^{-/-} mice.

The increased bone volume of Y2^{-/-}Y4^{-/-} mice was associated with a significant increase in bone turnover, with osteoblast surfaces, osteoclast surfaces and osteoid surfaces elevated as much as 100% compared to those of wt, Y4^{-/-} or Y2^{-/-} mice (Table 1).

TABLE 1
Histomorphometric analysis of bone cells in distal femoral
metaphysis in Y receptor knockout mice

| | | Osteoblast | | | |
|---|---|---|---|---|---|
| | Osteoblast | number (/mm | Osteoid | Osteoclast | Osteoclast |
| Mice | surface (% BS) | BS) | surface (% BS) | surface (% BS) | number (/mm) |
| Control | 10.4 ± 4.1 | 9.5 ± 2.5 | 12.7 ± 5.8 | 8.0 ± 2.3 | 4.2 ± 1.2 |
| Y2^{-/-} | 7.9 ± 2.7 | 7.3 ± 2.7 | 11.1 ± 4.4 | 6.4 ± 1.9 | 2.9 ± 0.6* |
| Y4^{-/-} | 10.1 ± 3.7 | 5.0 ± 1.5* | 12.0 ± 4.6 | 9.4 ± 2.9 | 4.5 ± 1.7 |
| Y2^{-/-}Y4^{-/-} | 18.6 ± 5.9* | 10.8 ± 3.4 | 23.2 ± 8.1* | 14.1 ± 5.8* | 4.1 ± 1.1 |

Data is presented as means ± standard deviation.
*P < 0.05 versus control.
BS, bone surface

The osteoblastic bone formation rate was significantly increased over control values in Y2^{-/-}Y4^{-/-} mice. This appears to be caused by an increase in mineral apposition rate, while there is no change in mineralizing surface, as shown in Table 2.

| | Mineralizing | Mineral | Bone |
|---|---|---|---|
| | surface (% | apposition rate | formation rate |
| Mice | BS) | (μm/day) | (μm²/day) |
| Control | 38.4 ± 12.7 | 1.1 ± 0.2 | 0.41 ± 0.1 |
| Y2^{-/-}Y4^{-/-} | 39.6 ± 7.2 | 1.4 ± 0.2* | 0.58 ± 0.2* |

Data is presented as means ± standard deviation.
*P < 0.05 versus control.
BS, bone surface

While the formation and rate of formation of cancellous bone was increased in Y2^{-/-}Y4^{-/-} mice, these mice also showed a significant decrease in the area and thickness of cortical bone compared to control, Y2^{-/-} and Y4^{-/-} mice (FIGS. 14A and 14B). This change was associated with a reduction in the periosteal diameter of the cortical shaft, with no change in the endosteal diameter (data not shown).

Analysis of Y2^{-/-}Y4^{-/-} double knockout mice indicates that the bone volume of these mice increases as a result of elevated bone formation, with an increased mineral apposition rate consistent with more active osteoblasts. Accordingly, these results shown that Y2 and Y4 act in a synergistic manner in the regulation of bone volume. Moreover, these results suggest that Y4 deficiency increases the sensitivity of a mouse to modulators of bone remodeling.

### EXAMPLE 5

### A Mouse Lacking Y1 Receptor Expression Displays Increased Cancellous Bone Volume

A targeting vector for the Y1 receptor gene is designed that allows the production of conditional (floxed, Y1^{lox/lox}) and germline (Y1^{-/-}) knockout mice in which the entire coding region of the Y1 receptor is removed (essentially as described in Howell et al, J. Neurochem. 86(3): 646-659, 2003). Briefly, the Y1 targeting construct is transfected into a mouse ES cell line. Clones are injected into C57BL/6 blastocysts. Chimeric offspring are crossed with oocyte-specific Cre-recombinase-expressing C57BL/6 mice (Schwenk et al. 1995) in order to obtain either heterozygotes carrying the floxed gene (Y1^{lox/+} mice) or heterozygotes carrying the Cre-recombinase gene with the floxed gene already deleted (Y1^{+/-} mice). Homozygous lines, both Y1^{-/-} and Y1^{lox/lox}, are generated by crossing the respective heterozygous animals. All further mice are maintained on this mixed C57BL/6-129SvJ background and, in the case of the Y1^{-/-} line, mice are selected that no longer contained the Cre-transgene.

As shown in FIG. 15, Y1^{-/-} mice show an increase in cancellous bone volume that is comparable to the levels observed in Y2^{-/-} mice and significantly greater than levels observed in Y4^{-/-} mice and wt mice.

### EXAMPLE 6

### Deficiency of Multiple Y Receptors Modulates Bone Remodeling

Mice deficient in Y1 and Y2 receptors are generated by crossing the Y2^{-/-} mice described in Example 1 and the Y1^{-/-} mice described in Example 5. Mice deficient in Y1 and Y4 receptors are generated by crossing the Y4^{-/-} mice described in Example 3 with the Y1^{-/-} mice described in Example 5. Mice deficient in Y1, Y2 and Y4 are generated by crossing the Y1^{-/-}Y2^{-/-} mice previously described with the Y4^{-/-} mice described in Example 3. The Y2^{-/-}Y4^{-/+} mice were described in Example 4.

As shown in FIG. 16 mice that are deficient in their expression of two or more of Y1, Y2 and Y4 have increased cancellous bone volume compared to Y4 deficient mice (which are approximately equivalent to wt mice). This increase in bone volume appears to be a result of increased trabecular thickness (FIG. 17). It is interesting that deficiency of more than one Y receptor induces greater trabecular thickness when compared to mice lacking a single Y receptor.

These results suggest that a compound that is capable of modulating the activity or expression of a single Y receptor (other than Y4) or a compound that is capable of modulating the activity or expression of multiple Y receptors will be of particular use in enhancing bone formation.

As was observed with the Y2^{-/-}Y4^{-/-} mice the targeted disruption of Y1, Y2, and Y4 led to a decrease in cortical thickness (FIG. 18a) and cortical area (FIG. 18b).

### EXAMPLE 7

### Y Receptor Modulated Bone Remodeling is More Pronounced in Male Mice

Male and female Y2^{-/-}Y4^{-/-} mice were analyzed to determine sex differences on the effect of Y receptor mediated bone remodeling. As shown in FIGS. 19A and 19B Y2^{-/-} male and female mice showed an increase in cancellous bone volume. Interestingly, Y2^{-/-}Y4^{-/-} male mice showed an synergistic increase in bone volume, in that the cancellous bone volume was increased in Y2^{-/-}Y4^{-/-} male mice above the level of Y2^{-/-} male mice. This effect was not observed in female mice.

In order to determine the cause of this effect osteoblast and osteoclast surfaces, mineralizing surface and rate of mineral apposition was determined in Y2^{-/-}Y4^{-/-} male and female mice. While the osteoclast and osteoblast surfaces were increased in Y2^{-/-} Y4^{-/-} mice, there was no significant difference between the results attained for male and female mice (FIG. 20).

In contrast, mineral apposition rate was found to be increased in both Y2^{-/-}Y4^{-/-} male and female mice, and the amount of increase in the mineral apposition rate in male mice was greater than that observed for female mice (FIG. 21). This suggests that the rate of bone formation is higher in male Y2^{-/-}Y4^{-/-} mice than in female Y2^{-/-}Y4^{-/-} mice.

The Y2^{-/-}Y4^{-/-} male mice also show a significant reduction in cortical mass as a result of reduced periosteal area, while this is not observed in female mice. This observation suggests a stimulation of cortical bone formation by NPY receptors.

### EXAMPLE 8

### Generation and Characterization of a Mouse Deficient in Y5 Receptor

Mice that are deficient in a Y5 receptor are generated essentially as described in Marsh et al, Nature Medicine, 4: 718-721, 1998. Briefly, a targeting construct is generated to replace the coding sequence of the Y5 receptor with a LacZ-Neo cassette. The Y5 targeting construct is transfected into a mouse ES cell line. Clones that have incorporated the targeting construct into the correct genomic location are injected into mouse blastocysts and the blastocysts injected into pseudopregnant female mice. Chimeric offspring are crossed wt mice in order to obtain heterozygotes carrying the deleted gene Y5+/−. Homozygous lines, Y5^{-/-}, are generated by crossing the heterozygous animals. All further mice are maintained on this mixed C57BL/6-129SvJ background.

All mice are then analyzed using the methods described in Examples 1 and 2 in order to determine the effect of the gene ablation of bone remodeling. All lines of mice are also mated to produce mice that lack all combinations of neuropeptide Y receptors in order to determine the effect of the loss of various combinations, in addition to all neuropeptide Y receptors.

### EXAMPLE 9

### Y Receptor Inhibition Replaces Bone Lost as a Result of Sex Hormone Deficiency

Hypogonadal bone loss is observed in women after menopause, and is associated with rapid loss of bone volume and associated strength. This bone loss appears to be caused by rapid loss of trabeculae and thinning of those trabeculae that remain. This form of bone loss is thought to contribute to the onset of osteoporosis in a large number of women.

Gonadectomized mice recapitulate many of the changes associated with hypogonadal bone loss in humans, including, for example, loss of bone mass, loss of cortical volume and loss of trabecular volume and number.

In order to assess the effect of Y receptors on hypogonadal bone loss Y1^{-/-, Y}2^{-/-} mice and wildtype mice are gonadectomized (10 males and 10 females of each genotype are gonadectomized) or sham operated at approximately 8 weeks of age (ie following the onset of bone remodeling). Following approximately 8 weeks gonadectomized mice (and age matched sham operated controls) are injected with a tetracycline label (ie 10 days prior to the termination of the experiment) and again 3 days prior to the termination of the experiment, enabling estimation of the rate and amount of bone formation.

Cancellous bone volume, trabecular number, trabecular volume, bone growth and rate of bone growth are determined using the methods described in Examples 1 and 2. Furthermore, estimates of osteoblast and osteoclast numbers and osteoblast and osteoclast surface are estimated with von Kossa stain and toluidine blue stain. Furthermore, plasma samples are analyzed to determine the success of the gonadectomy.

Protection of the Y receptor deficient mice from bone loss (and, in particular cancellous bone loss) indicates that inhibition of the expression of a Y receptor is protective against hypogonadal bone loss.

A second group of mice that carry a Y1^{lox/lox} or Y2^{lox/lox} mutation are gonadectomized (or sham operated) at 8 weeks of age. Hypogonadal bone loss is permitted to develop for 8 weeks, by which stage bones show dramatically reduced trabecular number and volume (Alexander et al., J. Bone and Min. Res., 16(9): 1665-1673, 2001). Following this period a portion of the mice are injected with a Cre expressing adenovirus (essentially as described in Example 2), while the remaining mice are injected with a GFP-expressing adenovirus. Following a period of 5 weeks, bones are collected and analyzed as previously described.

Results indicating an increase in trabecular bone volume or trabecular number following conditional silencing of a Y receptor is indicative of recovery from hypogonadal bone loss. Furthermore, increased mineral apposition rate indicates that modulation of the expression of a Y receptor increases the mass, volume and/or amount of cancellous bone thereby reducing the risk of osteoporotic fractures.

### EXAMPLE 10

### Y Receptor Inhibition Protects Against Age Related Bone Loss

Y1^{-/-}, Y2^{-/-} and wt mice are aged for a period of approximately 8 weeks, 36 weeks and 8 weeks. 10 days prior to termination of the experiment mice are injected with tetracycline labels and this process is repeated 3 days prior to termination of the experiment. Lumbar and caudal vertebrae, distal femur and proximal tibia are collected and analyzed to determine the effects of ageing on cancellous bone volume, trabecular number, trabecular volume, bone growth, bone growth rate, osteoblast and osteoclast numbers and osteoblast and osteoclast surfaces. These parameters are estimated using methods described previously (Examples 1 and 2).

Mice are assessed to determine the effect of Y1 or Y2 receptor silencing on changes in bone phenotypes normally associated with ageing. For example, aged mice display a loss of cancellous bone volume, reduced bone volume and a reduction in the number of trabeculae. Accordingly, a reduction in any of these phenotypes in a Y1^{-/-} or Y2^{-/-} mouse is indicative of the protective effects of a modulator of Y receptor activity.

A second group of mice that carry a Y1^{lox/lox} or Y2^{lox/lox} mutation are aged until they are approximately 9 months of age. Following this period a portion of the mice are injected with a Cre expressing adenovirus (essentially as described in Example 2), while the remaining mice are injected with a GFP-expressing adenovirus. Mice are then analyzed for the various parameters of bone remodeling previously described.

By 9 months of age mice have developed a clear loss of cancellous bone volume in association with loss of trabecular number. Accordingly, an increase in the number of trabeculae or in cancellous bone volume in the conditional knockout mice over control mice indicates the protective effect of suppression of Y receptors.

### EXAMPLE 11

### The Effect of Y Receptor Deficiency on Adiposity

In order to assess the effect of Y receptor expression on adiposity the adipocyte area in marrow of wildtype and Y receptor KO mice is determined.

Histomorphometry is used to quantify adipocytes in marrow of bone sections from wildtype and Y receptor deficient mice essentially as described by Verma et al, J. Clin. Pathol., 55:693-698, 2002 (incorporated herein by reference). Sagittal sections of distal femur and/or vertebrae (5 μm) are stained by Von Kossa and haematoxylin and eosin methods. Semi-automated image analysis using Bioquant system is used to determine areas of adipocyte and hematopoietic/stromal cells, and the ratios of these two areas is compared between mouse lines.

Furthermore, adipocyte numbers in marrow of wildtype and Y receptor deficient mice are assessed. Marrow from tibias and femurs of wildtype and germline Y receptor deficient mice is collected at 8 weeks and 16 weeks of age, essentially as described by Satomura et al., J. Cell. Biochem, 78: 391-403, 2000 (incorporated herein by reference). Briefly, the epiphyses are removed and marrow tissue and flushed from the shaft using cell culture medium. A single cell suspension is prepared by aspirating marrow first through a 20- and then through a 23-gauge needle, followed by straining through a 700□m□sieve.

Mature lipid-containing adipocytes are separated from the total cell population by low speed centrifugation (Rodell J. Biol. Chem. 239: 375-381, 1964). Cells are stained with Oil Red O and the number of adipocytes estimated microscopically in wildtype and Y receptor deficient samples using hemacytometer. Raw data is Normalized against total viable cell count using trypan blue exclusion with viable cell numbers determined using a hemacytometer.

The total adipocyte cell preparation (prepared previously) is stained with Nile Red as described by Sen et al., J. Cell. Biochem., 81: 312-319, 2001 (incorporated herein by reference). The total percentage of lipid-containing cells is then estimated in wildtype and Y receptor deficient cell preparations using flow cytometry, essentially as described by Sen et al., J. Cell. Biochem., 81: 312-319, 2001.

### EXAMPLE 12

### The Effect of Y Receptor Deficiency on Differentiation of Mesenchymal Stem Cells

The relative numbers of adipocytic and osteoblastic progenitors in marrow of wildtype and Y receptor KO mice are determined by limiting dilution analysis.

Bone marrow is collected from tibias and femurs of wildtype and germline Y receptor deficient mice at 8 weeks and 16 weeks of age. Marrow cell suspensions are prepared essentially as described in Satomura et al., J. Cell. Biochem, 78: 391-403, 2000. Cultures for adipogenic and osteoblastic differentiation are then prepared.

Osteoblastic differentiation. Cell cultures are incubated in α-MEM supplemented with 20% heat inactivated fetal bovine serum with 50 μM ascorbic acid and 10 nM β-glycerophosphate, with or without 10 nM dexamethasone to induce osteoblastic differentiation, a modification of the culture conditions described by Drissi et al., Cancer Res., 59: 3705-3711, 1999 (incorporated herein by reference). For limiting dilution analysis of osteoprogenitor cell number, cells are plated in 96 well plates at densities ranging from 10³ to 10⁵ cells/well and grown until appearance of robust mineralized nodules. Wells are then fixed and stained by the Von Kossa technique and visualized under low power microscopy. The frequency of osteoprogenitor cells is determined by quantifying the fraction of wells not containing bone nodules at each cell density tested. Osteoprogenitor cell number is then determined, essentially as described by Aubin J. Cell. Biochem., 72: 396-410, 1999 (incorporated herein by reference).

Adipogenic differentiation. Cells are cultured to 3 to 7 days post confluence and then incubated in α-MEM supplemented with FCS, dexamethasone, insulin and IBMX, essentially as described by Murphy et al, Arthritis and Rheumatism, 46(3): 704-713, 2002 (incorporated herein by reference). For limiting dilution analysis, cells are plated in 96 well plates at densities of 10³ to 10⁵ cells/well and grown until adipocytes with large lipid vacuoles are present. Wells are individually scored for presence of adipocytes. The frequency of adipocytic progenitor cells is determined by quantifying the fraction of wells not containing adipocytes at each cell density tested. Adipocytic progenitor cell number is then determined, applying the principles and formulas described by Aubin J. Cell. Biochem., 72: 396-410, 1999.

Any change in the relative numbers of adipocyte-forming and osteoblast-forming progenitors derived from wildtype or Y receptor deficient mice is indicative of the effect of Y receptor activity on the differentiation of osteoblasts and adipocytes.

Additionally, the differentiation responses of marrow-derived mesenchymal stem cells as a result of Y receptor activation is determined

Suspensions of marrow cells from wildtype and KO mouse marrow samples are prepared as described supra and adipogenic and osteogenic cultures as described supra.

96-well cultures plated at to 10⁵ cells/well are treated with increasing dosages of Y receptor agonist or antagonist prior to or at initiation of adipogenic or osteoblastic differentiation protocol or during differentiation period. The number of osteoblastic and adipocyte progenitor cells are then determined essentially as described in Aubin J. Cell. Biochem., 72: 396-410, 1999.

A change in MSC-derived colonies of the two lineages in the wildtype mice but not in Y receptor deficient mice after treatment with Y receptor agonist or antagonist indicates that at least one Y receptor modulates the formation of osteoblasts and/or adipocytes.

Additionally, the relative numbers of adipocytic and osteoblastic progenitors in peripheral fat of wildtype and Y receptor KO mice is determined by limiting dilution analysis.

Epididymal or retroperitoneal fat pads are removed from wildtype and Y receptor deficient mice and minced. Adipose stromal cell populations are prepared essentially as described by Zuk et al, Mol. Biol. Cell, 13: 4279-4295, 2002 (incorporated herein by reference). Essentially, fat pads are dissociated, washed extensively with sterile phosphate buffered saline, collagenase digested and centrifuged at low speed to separate the stromal cells from lipid-containing adipocytes. The resuspended cell pellet is filtered through a 70 μm mesh filter. The filtered cells are cultured to induce adipocytic or osteoblastic differentiation. Cultures for adipogenic and osteoblastic differentiation are then prepared.

Osteoblastic differentiation. Cell cultures are incubated in α-MEM supplemented with 20% heat inactivated fetal bovine serum with 50 μM ascorbic acid and 10 mM β-glycerophosphate, with or without 10 nM dexamethasone to induce osteoblastic differentiation, a modification of the culture conditions described by Drissi et al., Cancer Res., 59: 3705-3711, 1999 (incorporated herein by reference). For limiting dilution analysis of osteoprogenitor cell number, cells are plated in 96 well plates at densities ranging from 10³ to 10⁵ cells/well and grown until appearance of robust mineralized nodules. Wells are then fixed and stained by the Von Kossa technique and visualized under low power microscopy. The frequency of osteoprogenitor cells is determined by quantifying the fraction of wells not containing bone nodules at each cell density tested. Osteoprogenitor cell number is then determined, essentially as described by Aubin J. Cell. Biochem., 72: 396-410, 1999 (incorporated herein by reference).

Adipogenic differentiation. Cells are cultured to 3 to 7 days post confluence and then incubated in α-MEM supplemented with FCS, dexamethasone, insulin and IBMX, essentially as described by Murphy et al, Arthritis and Rheumatism, 46(3): 704-713, 2002 (incorporated herein by reference). For limiting dilution analysis, cells are plated in 96 well plates at densities of 10³ to 10⁵ cells/well and grown until adipocytes with large lipid vacuoles are present. Wells are individually scored for presence of adipocytes. The frequency of adipocytic progenitor cells is determined by quantifying the fraction of wells not containing adipocytes at each cell density tested. Adipocytic progenitor cell number is then determined, applying the principles and formulas described by Aubin J. Cell. Biochem., 72: 396-410, 1999.

Adipogenic and osteoblastic differentiation responses of peripheral fat-derived mesenchymal stem cells to Y receptor activation is also determined. Briefly, peripheral adipose stromal cells are prepared essentially as described by Zuk et al, Mol. Biol. Cell, 13: 4279-4295, 2002 and adipocyte or osteoblast progenitor cells cultured as described supra.

96-well cultures plated at 10³ to 10⁵ cells/well are treated with increasing dosages of Y receptor agonist or antagonist prior to or at initiation of adipogenic or osteoblastic differentiation protocol or during differentiation period. The number of osteoblastic and adipocyte progenitor cells are then determined essentially as described in Aubin J. Cell. Biochem., 72: 396-410, 1999.

### EXAMPLE 13

### Effect of Y Receptors on Adiposity

Y2^{-/-}Y4^{-/-} mice were assessed for any changes in adiposity. As shown in FIG. 23 both male and female Y2^{-/-}Y4^{-/-} showed significantly reduced body weight compared to wild-type animals. Furthermore, the food intake was significantly increased compared to control, Y2^{-/-} and Y4^{-/-} mice (not shown).

The weight of white adipose tissue and brown adipose tissue compared to body weight was also significantly reduced in Y2^{-/-}Y4^{-/-} compared with wild-type controls (data not shown). This suggests that the Y2^{-/-}Y4^{-/-} mice have an increased metabolic rate compared to wild-type mice.

1-105. (canceled)

106. A method of determining a compound that is a modulator of Y receptor associated differentiation of a mesenchymal stem cell (MSC) or bone marrow stromal cell (BMSC) into an osteoblast-type cell comprising:
(i) culturing a MSC or BMSC in the presence of a candidate compound;
(ii) culturing a MSC or BMSC in the absence of the candidate compound; and
(iii) determining Y receptor activity and/or expression and the number of differentiated osteoblast-type cells at (i) and (ii), wherein a modified number of osteoblast-type cells and a modified Y receptor activity and/or expression at (i) and (ii) indicates that the compound is a modulator of Y receptor associated differentiation of an osteoblast-type cell.

107. A method of determining a compound that is a modulator of Y receptor associated differentiation of a mesenchymal stem cell (MSC) or bone marrow stromal cell (BMSC) into an adipocyte-type cell comprising:
(i) culturing a MSC or BMSC in the presence of a candidate compound;
(ii) culturing a MSC or BMSC in the absence of the candidate compound; and
(iii) determining Y receptor activity and/or expression and the number of differentiated adipocyte-type cells at (i) and (ii), wherein a modified number of adipocyte-type cells and a modified Y receptor activity and/or expression at (i) and (ii) indicates that the compound is a modulator of Y receptor associated differentiation of an adipocyte-type cell.

108-121. (canceled)

122. A non-naturally occurring transformed animal having reduced expression of multiple Y receptors in a cell or tissue by virtue of carrying insertions in multiple Y receptor-encoding genes wherein said animal has modulated bone remodeling activity, modulated bone growth activity or modulated adiposity compared to an otherwise isogenic animal that does not carry the insertions.

123-124. (canceled)

125. The non-naturally occurring transformed animal according to claim 122 wherein the multiple Y receptor encoding genes carrying the insertions are selected from the group consisting of:
(i) a Y1 receptor and a Y2 receptor;
(ii) a Y1 receptor and a Y5 receptor;
(iii) a Y1 receptor and a Y7 receptor;
(iv) a Y2 receptor and a Y5 receptor;
(v) a Y2 receptor and a Y7 receptor;
(vi) a Y5 receptor and a Y7 receptor;
(vii) a Y1 receptor and a Y2 receptor and a Y5 receptor;
(viii) a Y1 receptor and a Y2 receptor and a Y7 receptor;
(ix) a Y1 receptor and a Y5 receptor and a Y7 receptor; and
(x) a Y1 receptor and a Y2 receptor and a Y5 receptor and a Y7 receptor.

126. The non-naturally occurring transformed animal according to claim 122 wherein the multiple Y receptor encoding genes carrying the insertions are selected from the group consisting of:
(i) a Y1 receptor and a Y2 receptor;
(ii) a Y2 receptor and a Y4 receptor; and
(iii) a Y1 receptor and a Y2 receptor and a Y4 receptor.

127. The non-naturally occurring transformed animal according to claim 122 wherein the animal is a mouse.

128. A progeny animal of the non-naturally occurring transformed animal according to claim 122 wherein said progeny animal has reduced expression of multiple Y receptors in a cell or tissue by virtue of carrying insertions in multiple Y receptor-encoding genes.

129-138. (canceled)

139. A method of treatment of aberrant bone remodeling in a subject in need thereof comprising isolating a mesenchymal stem cell (MSC) or bone marrow stromal cell (BMSC) from a human or animal subject, treating the MSC or BMSC with a compound that modulates Y receptor associated differentiation under conditions sufficient to induce differentiation of the MSC or BMSC into an osteoblast type cell and introducing the osteoblast type cell into the subject in need of treatment.

140-150. (canceled)

151. A method comprising administering a compound to the non-naturally occurring transformed animal of claim 122 and determining bone remodeling activity, bone growth or adiposity of the animal to thereby determine an effect of the compound on bone remodeling activity, bone growth or adiposity.

152. The method of claim 151 wherein the non-naturally occurring transformed animal has reduced expression of a Y4 receptor in a cell or tissue by virtue of carrying an insertion in the Y4 receptor-encoding gene.


SEQUENCE LISTING (20 sequences)

SEQ ID NO: 1 - DNA, 1180 bp, human neuropeptide Y1 receptor
  CDS (7)..(1161)
aagcttatgaattcaacattattttcccaggttgaaaatcattcagtccactctaatttctcagagaagaatgcccagcttctggcttttgaaaatgatgattgtcatctgcccttggccatgatatttaccttagctcttgcttatggagctgtgatcattcttggtgtctctggaaacctggccttgatcataatcatcttgaaacaaaaggagatgagaaatgttaccaacatcctgattgtgaacctttccttctcagacttgcttgttgccatcatgtgtctcccctttacatttgtctacacattaatggaccactgggtctttggtgaggcgatgtgtaagttgaatccttttgtgcaatgtgtttcaatcactgtgtccattttctctctggttctcattgctgtggaacgacatcagctgataatcaaccctcgagggtggagaccaaataatagacatgcttatgtaggtattgctgtgatttgggtccttgctgtggcttcttctttgcctttcctgatctaccaagtaatgactgatgagccgttccaaaatgtaacacttgatgcgtacaaagacaaatacgtgtgctttgatcaatttccatcggactctcataggttgtcttataccactctcctcttggtgctgcagtattttggtccactttgttttatatttatttgctacttcaagatatatatacgcctaaaaaggagaaacaacatgatggacaagatgagagacaataagtacaggtccagtgaaaccaaaagaatcaatatcatgctgctctccattgtggtagcatttgcagtctgctggctccctcttaccatctttaacactgtgtttgattggaatcatcagatcattgctacctgcaaccacaatctgttattcctgctctgccacctcacagcaatgatatccacttgtgtcaaccccatattttatgggttcctgaacaaaaacttccagagagacttgcagttcttcttcaacttttgtgatttccggtctcgggatgatgattatgaaacaatagccatgtccacgatgcacacagatgtttccaaaacttctttgaagcaagcaagcccagtcgcatttaaaaaaatcaacaacaatgatgataatgaaaaaatctgaaactacttatagctctaga

SEQ ID NO: 2 - protein, 384 aa, human neuropeptide Y1 receptor
MNSTLFSQVENHSVHSNFSEKNAQLLAFENDDCHLPLAMIFTLALAYGAVIILGVSGNLALIIIILKQKEMRNVTNILIVNLSFSDLLVAIMCLPFTFVYTLMDHWVFGEAMCKLNPFVQCVSITVSIFSLVLIAVERHQLIINPRGWRPNNRHAYVGIAVIWVLAVASSLPFLIYQVMTDEPFQNVTLDAYKDKYVCFDQFPSDSHRLSYTTLLLVLQYFGPLCFIFICYFKIYIRLKRRNNMMDKMRDNKYRSSETKRINIMLLSIVVAFAVCWLPLTIFNTVFDWNHQIIATCNHNLLFLLCHLTAMISTCVNPIFYGFLNKNFQRDLQFFFNFCDFRSRDDDYETIAMSTMHTDVSKTSLKQASPVAFKKINNNDDNEKI

SEQ ID NO: 3 - DNA, 3747 bp, human neuropeptide Y2 receptor
  CDS (496)..(1638)
gaattcggccgctgagagaccctggacactgttcctgctccctcgccaccaaaacttctcctccagtcccctcccctgcaggaccatcgcccgcagcctctgcacctgttttcttgtgtttaagggtggggtttgcccccctccccacgctcccatctctgatcctcccaccttcacccgcccaccccgcgagtgagtgcggtgcccaggcgcgcttggcctgagaggtcggcagcagacccggcagcgccaaccgcccagccgctctgactgctccggctgcccgcccgcgcggcgcgggctgtcctggaccctaggaggggacggaaccggacttgcctttgggcaccttccagggccctctccaggtcggctggctaatcatcggacagacggactgcacacatcttgtttccgcgtctccgcaaaaacgcgaggtccaggtcagttgtagactcttgtgctggttgcaggccaagtggacctgtactgaaaatgggtccaataggtgcagaggctgatgagaaccagacagtggaagaaatgaaggtggaacaatacgggccacaaacaactcctagaggtgaactggtccctgaccctgagccagagcttatagatagtaccaagctgattgaggtacaagttgttctcatattggcctactgctccatcatcttgcttggggtaattggcaactccttggtgatccatgtggtgatcaaattcaagagcatgcgcacagtaaccaactttttcattgccaatctggctgtggcagatcttttggtgaacactctgtgtctaccgttcactcttacctataccttaatgggggagtggaaaatgggtcctgtcctgtgccacctggtgccctatgcccagggcctggcagtacaagtatccacaatcaccttgacagtaattgccctggaccggcacaggtgcatcgtctaccacctagagagcaagatctccaagcgaatcagcttcctgattattggcttggcctggggcatcagtgccctgctggcaagtcccctggccatcttccgggagtattcgctgattgagatcattccggactttgagattgtggcctgtactgaaaagtggcctggcgaggagaagagcatctatggcactgtctatagtctttcttccttgttgatcttgtatgttttgcctctgggcattatatcattttcctacactcgcatttggagtaaattgaagaaccatgtcagtcctggagctgcaaatgaccactaccatcagcgaaggcaaaaaaccaccaaaatgctggtgtgtgtggtggtggtgtttgcggtcagctggctgcctctccatgccttccagcttgccgttgacattgacagccaggtcctggacctgaaggagtacaaactcatcttcacagtgttccacattatcgccatgtgctccacttttgccaatccccttctctatggctggatgaacagcaactacagaaaggctttcctctcggccttccgctgtgagcagcggttggatgccattcactctgaggtgtccgtgacattcaaggctaaaaagaacctggaggtcagaaagaacagtggccccaatgactctttcacagaggctaccaatgtctaaggaagctgtggtgtgaaaatgtatggatgaattctgaccagagctatgaatctggttgatggcggctcacaagtgaaaactgatttcccattttaaagaagaagtggatctaaatggaagcatctgctgtttaattcctggaaaactggctgggcagagcctgtgtgaaaatactggaattcaaagataaggcaacaaaatggtttacttaacagttggttgggtagtaggttgcattatgagtaaaagcagagagaagtacttttgattattttcctggagtgaagaaaacttgaacaagaaattggtattatcaaagcattgctgagagacggtgggaaaataagttgactttcaaatcacgttaggacctggattgaggaggtgtgcagttcgctgctccctgcttggcttatgaaaacaccactgaacagaaatttctccagggagccacaggctctccttcatcgcattttgatttttttgttcattctctagacaaaatccatcagggaatgctgcaggaaacgattgccaactatacgaatggcttcgaggagataaactgaaatttgctatataattaatattttggcagatgataggggaactcctcaacactcagtgggccaattgttcttaaaaccaattgcacgtttggtgaaagtttcttcaactctgaatcaaaagctgaaattctcagaattacaggaaatgcaaaccatcatttaatttctaatttcaagttacatccgctttatggagatactatttagataacaagaatacaacttgatacttttattgttatacctttttgaacatgtatgatttctgttgttattcctattggagctaagtttgtctacactaaaatttaaatcagactagagaataatttttgtggcatgttgtaacatttcacagtatttacaagctatttttgcacaggtacatagctctcatgtatttaaagaacactgcagtgttattttctttgaaattcatcctccacggacccattcatactaaataaaacaatgtaattacattaaaatggacctatctgtaagaggtactaaaaacactggattcatttcatcttgcaaatgttgtatttcaaaccagtttcacataagttatttgtcttcttttcaaaataattagctatatttttatataatatgaatatatacataaaaattgtttctataaattgtagaacatagatgctacagtattttttatttaattatattatgaataaaattgttatttcaatagtacccaaccaaagatgcttaaaaaccttctatgttcataaaaaataacaactgagatgttaaaatagtcatacgtctttagatgctattaaagtttcattagtcatatttttgtaaatatgacagaatttgtgaatatatttttaaagcaaaaaacttcaacatgcatatgatatatagttacaacattaattttatgaactggagagctttactttgtggatatatttaaaattcatattatagctcctattaaattccttccatgatagatataaaggactggtttttaagtgcactgcacttctggaatactgaaaaagaatgaaaacaatatgttagattaggtgtaagactttaagaagcgaacaaaaagtaatgtatatctgtaatatataatcaaatgattcatttttctgttagactaggcaaattgttcaaaaataacctttttgtcttttaagtagcagtcactttgcttaagatgctaatagaaaactgtggttaaagatttaccctccctcttggtgaattattacactgtaagaaatgtatatgctactgtgttacatgttgtattagtaaattattagaatccaattaatgattcaattaacatatatcttatccaattcattatgtcaattcattaataaaataccttttatgtagaggctttatgttgcaattaaaaagttgggaaaatgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 4 - protein, 381 aa, human neuropeptide Y2 receptor
MGPIGAEADENQTVEEMKVEQYGPQTTPRGELVPDPEPELIDSTKLIEVQVVLILAYCSIILLGVIGNSLVIHVVIKFKSMRTVTNFFIANLAVADLLVNTLCLPFTLTYTLMGEWKMGPVLCHLVPYAQGLAVQVSTITLTVIALDRHRCIVYHLESKISKRISFLIIGLAWGISALLASPLAIFREYSLIEIIPDFEIVACTEKWPGEEKSIYGTVYSLSSLLILYVLPLGIISFSYTRIWSKLKNHVSPGAANDHYHQRRQKTTKMLVCVVVVFAVSWLPLHAFQLAVDIDSQVLDLKEYKLIFTVFHIIAMCSTFANPLLYGWMNSNYRKAFLSAFRCEQRLDAIHSEVSVTFKAKKNLEVRKNSGPNDSFTEATNV

SEQ ID NO: 5 - DNA, 2260 bp, human neuorpeptide Y4 receptor
  CDS (660)..(1787)
ggatccccgaggctttagctctatcttgaagggaatggggtgccgtggaaggacacaagcaatcctgtgctgagtggcatttattgctgtctccattcagataaaatgtgacaaggggcattctacccatgttccctgaacaattccgtcactaaagtctgcatacagcactccaacttttttatcatcagaacagcctcatcctgaggcggagggaattagtagaacaagttctggcattagatgacctgagtttgtaactgggcctgttactaattagctgtgtgactatgggcaagttgctttccatctcagggtcttagattcctcatctatgaaacggattgataatatctacctctaggaataaactagctaatagacgaaagtgctgagcacagtacccagcatgcaacaagtgcttctaacaaggtagcaatcattggatgccaatgtctcaccatgagccttaggtaatagccctccagataccagccttgggttggaagcagctgagagcctttctacccggccctgctcctctctggattctaccctgggcatgatccctgagtattgtttgtctgtttgccttgtagggcgtcatccctcaagtgtatcacttagttcaagagtcctggaatcttttcacatccactatgaacacctctcacctcctggccttgctgctcccaaaatctccacaaggtgaaaacagaagcaaacccctgggcaccccatacaacttctctgaacattgccaggattccgtggacgtgatggtcttcatcgtcacttcctacagcattgagactgtcgtgggggtcctgggtaacctctgcctgatgtgtgtgactgtgaggcagaaggagaaagccaacgtgaccaacctgcttatcgccaacctggccttctctgacttcctcatgtgcctcctctgccagccgctgaccgccgtctacaccatcatggactactggatctttggagagaccctctgcaagatgtcggccttcatccagtgcatgtcggtgacggtctccatcctctcgctcgtcctcgtggccctggagaggcatcagctcatcatcaacccaacaggctggaagcccagcatctcacaggcctacctggggattgtgctcatctgggtcattgcctgtgtcctctccctgcccttcctggccaacagcatcctggagaatgtcttccacaagaaccactccaaggctctggagttcctggcagataaggtggtctgtaccgagtcctggccactggctcaccaccgcaccatctacaccaccttcctgctcctcttccagtactgcctcccactgggcttcatcctggtctgttatgcacgcatctaccggcgcctgcagaggcaggggcgcgtgtttcacaagggcacctacagcttgcgagctgggcacatgaagcaggtcaatgtggtgctggtggtgatggtggtggcctttgccgtgctctggctgcctctgcatgtgttcaacagcctggaagactggcaccatgaggccatccccatctgccacgggaacctcatcttcttagtgtgccacttgcttgccatggcctccacctgcgtcaacccattcatctatggctttctcaacaccaacttcaagaaggagatcaaggccctggtgctgacttgccagcagagcgcccccctggaggagtcggagcatctgcccctgtccacagtacatacggaagtctccaaagggtccctgaggctaagtggcaggtccaatcccatttaaccaggtctaggtcttctccctgccatgtcccttgccaggctcttccacttagctaagtgggcacactgcaagctggggtggcaccccagcattcctggctttctggggtccagataggctggcaagagctgtttttgcatccatttgcatcgtgaagactggcattttgatacttcagctgtttgttcctgggagaattctgagcacagattccagaggtcacagtaagccttgcagcttgagctgaaagatgccagagccggagatgtctgctggcagcaggcagggttcattctggtgacacagcaacagatccctggcctgggaacccagggatttcacctccaccagtgagaccacggggccactgtggggtgagggaaggagcgcttggagtcagagctctagaccttggtcaactcttcacctctgtgagagatggtgtgtggaggtgcttcagaagtaaagaattc

SEQ ID NO: 6 - protein, 375 aa, human neuorpeptide Y4 receptor
MNTSHLLALLLPKSPQGENRSKPLGTPYNFSEHCQDSVDVMVFIVTSYSIETVVGVLGNLCLMCVTVRQKEKANVTNLLIANLAFSDFLMCLLCQPLTAVYTIMDYWIFGETLCKMSAFIQCMSVTVSILSLVLVALERHQLIINPTGWKPSISQAYLGIVLIWVIACVLSLPFLANSILENVFHKNHSKALEFLADKVVCTESWPLAHHRTIYTTFLLLFQYCLPLGFILVCYARIYRRLQRQGRVFHKGTYSLRAGHMKQVNVVLVVMVVAFAVLWLPLHVFNSLEDWHHEAIPICHGNLIFLVCHLLAMASTCVNPFIYGFLNTNFKKEIKALVLTCQQSAPLEESEHLPLSTVHTEVSKGSLRLSGRSNPI

SEQ ID NO: 7 - DNA, 1423 bp, human neuropeptide Y5 receptor
  CDS (71)..(1408)
gaaaggctatcggtaacaactgacctgccacaaagttagaagaaaggattgattcaagaaagactataatatggatttagagctcgacgagtattataacaagacacttgccacagagaataatactgctgccactcggaattctgatttcccagtctgggatgactataaaagcagtgtagatgacttacagtattttctgattgggctctatacatttgtaagtcttcttggctttatggggaatctacttattttaatggctctcatgaaaaagcgtaatcagaagactacggtaaacttcctcataggcaatctggccttttctgatatcttggttgtgctgttttgctcacctttcacactgacgtctgtcttgctggatcagtggatgtttggcaaagtcatgtgccatattatgccttttcttcaatgtgtgtcagttttggtttcaactttaattttaatatcaattgccattgtcaggtatcatatgataaaacatcccatatctaataatttaacagcaaaccatggctactttctgatagctactgtctggacactaggttttgccatctgttctccccttccagtgtttcacagtcttgtggaacttcaagaaacatttggttcagcattgctgagcagcaggtatttatgtgttgagtcatggccatctgattcatacagaattgcctttactatctctttattgctagttcagtatattctgcccttagtttgtcttactgtaagtcatacaagtgtctgcagaagtataagctgtggattgtccaacaaagaaaacagacttgaagaaaatgagatgatcaacttaactcttcatccatccaaaaagagtgggcctcaggtgaaactctctggcagccataaatggagttattcattcatcaaaaaacacagaagaagatatagcaagaagacagcatgtgtgttacctgctccagaaagaccttctcaagagaaccactccagaatacttccagaaaactttggctctgtaagaagtcagctctcttcatccagtaagttcataccaggggtccccacttgctttgagataaaacctgaagaaaattcagatgttcatgaattgagagtaaaacgttctgttacaagaataaaaaagagatctcgaagtgttttctacagactgaccatactgatattagtatttgctgttagttggatgccactacaccttttccatgtggtaactgattttaatgacaatcttatttcaaataggcatttcaagttggtgtattgcatttgtcatttgttgggcatgatgtcctgttgtcttaatccaattctatatgggtttcttaataatgggattaaagctgatttagtgtcccttatacactgtcttcatatgtaataattctcactgttt

SEQ ID NO: 8 - protein, 445 aa, human neuropeptide Y5 receptor
MDLELDEYYNKTLATENNTAATRNSDFPVWDDYKSSVDDLQYFLIGLYTFVSLLGFMGNLLILMALMKKRNQKTTVNFLIGNLAFSDILVVLFCSPFTLTSVLLDQWMFGKVMCHIMPFLQCVSVLVSTLILISIAIVRYHMIKHPISNNLTANHGYFLIATVWTLGFAICSPLPVFHSLVELQETFGSALLSSRYLCVESWPSDSYRIAFTISLLLVQYILPLVCLTVSHTSVCRSISCGLSNKENRLEENEMINLTLHPSKKSGPQVKLSGSHKWSYSFIKKHRRRYSKKTACVLPAPERPSQENHSRILPENFGSVRSQLSSSSKFIPGVPTCFEIKPEENSDVHELRVKRSVTRIKKRSRSVFYRLTILILVFAVSWMPLHLFHVVTDFNDNLISNRHFKLVYCICHLLGMMSCCLNPILYGFLNNGIKADLVSLIHCLHM

SEQ ID NO: 9 - DNA, 1903 bp, human neuropeptide Y7 receptor
  CDS (369)..(1595)
ctcgagatccattgtgctctaaaggcctcctgagtagctgggactacaggcgcccgccaccacgcctggctaatttttttgtatttttagtagggacggcgtttcactgtgttagccagatggtctccatctcccgacctcgtgatccacccacctcggcctcccaaagtgctgggattacaggcgtgagaccgcgcccggccaatttcctttcttagttgcctctgcccacctcttctcttctgcttccatattacaggtttcctcagttgcgaaattaggatgttaattatagcttttgacatacaagaaacatcaaaaagattgaatgtcttaataagagtgaagcatgtagatcagtgactgctatgttcatcatgaatgagaaatgggacacaaactcttcagaaaactggcatcccatctggaatgtcaatgacacaaagcatcatctgtactcagatattaatattacctatgtgaactactatcttcaccagcctcaagtggcagcaatcttcattatttcctactttctgatcttctttttgtgcatgatgggaaatactgtggtttgctttattgtaatgaggaacaaacatatgcacacagtcactaatctcttcatcttaaacctggccataagtgatttactagttggcatattctgcatgcctataacactgctggacaatattatagcaggatggccatttggaaacacgatgtgcaagatcagtggattggtccagggaatatctgtcgcagcttcagtctttacgttagttgcaattgctgtagataggttccagtgtgtggtctacccttttaaaccaaagctcactatcaagacagcgtttgtcattattatgatcatctgggtcctagccatcaccattatgtctccatctgcagtaatgttacatgtgcaagaagaaaaatattaccgagtgagactcaactcccagaataaaaccagtccagtctactggtgccgggaagactggccaaatcaggaaatgaggaagatctacaccactgtgctgtttgccaacatctacctggctcccctctccctcattgtcatcatgtatggaaggattggaatttcactcttcagggctgcagttcctcacacaggcaggaagaaccaggagcagtggcacgtggtgtccaggaagaagcagaagatcattaagatgctcctgattgtggccctgctttttattctctcatggctgcccctgtggactctaatgatgctctcagactacgctgacctttctccaaatgaactgcagatcatcaacatctacatctacccttttgcacactggctggcattcggcaacagcagtgtcaatcccatcatttatggtttcttcaacgagaatttccgccgtggtttccaagaagctttccagctccagctctgccaaaaaagagcaaagcctatggaagcttataccctaaaagctaaaagccatgtgctcataaacacatctaatcagcttgtccaggaatctacatttcaaaaccctcatggggaaaccttgctttataggaaaagtgctgaaaaccccaacaggaattagtgatggaagaattaaaagaaactactaacagcagtgagatttaaaaagagctagtgtgataatcctaactctactacgcattatatatttaaatccattgctttttgtggctttgcacttcaaatttttcaaagaatgttctaaataaaacatttactgaaagccctctctggcaaaaaaattaaaaataaacaaaaatggtcataagatcataaacaatcttatgttgtataaaaatacgtagagtgacttagacatgtttgcatgaataaatatatttctagagaacagttaaaaaaaaaaaaaaa

SEQ ID NO: 10 - protein, 408 aa, human neuropeptide Y7 receptor [sequence omitted]

SEQ ID NO: 11 - DNA, 1980 bp, mouse neuropeptide Y1 receptor
  CDS (135)..(1283)
agggaatgaagaactgagaattatcttggtgaatggattcaaatatatggaataagagtatgctgaagatttgatccgttttgaagaactataactgtccatttatctaatcggtaacaacaaaacataaaaaaatgaactcaactctgttctccaaggttgaaaatcactcaattcactataatgcctcagagaattctccacttctggcttttgaaaatgatgactgccacctgcccttggctgtgatattcaccttggctctcgcttatggggcggtgattattcttggcgtctctggaaacctggcattgatcataatcattctgaaacagaaggagatgagaaatgtcaccaacattctgatcgtgaacctctccttctcagacttgctcgttgcggtcatgtgtctcccgttcacttttgtatatacactgatggaccactgggtcttcggggagaccatgtgcaaactgaatccctttgtacagtgtgtctccatcacagtatccattttctcgctggttctcatcgctgtggaacggcatcagctaatcatcaacccaagagggtggagaccaaacaatagacatgcttacataggcattactgtcatttgggtccttgcagtggcttcttctctgccctttgtgatctatcaaattctgaccgacgagcccttccaaaatgtgtcacttgcggcgttcaaggacaagtatgtgtgctttgacaaattcccatctgactctcacaggctgtcttacacgactctcctcctggtgctgcagtatttcggcccactctgctttatattcatatgctacttcaagatatacattcgcttgaaaaggagaaacaacatgatggacaagatccgggacagtaagtacaggtccagtgagaccaagcgaatcaacatcatgctgctctccattgtggtcgccttcgccgtctgctggctgccccttaccatcttcaacactgtgttcgactggaaccaccagatcattgccacctgcaaccacaatctgctgtttctgctctgtcacctcaccgccatgatctccacctgcgtcaaccccatcttttatggattcctgaacaaaaatttccagagagacttgcagttcttcttcaacttttgtgacttccggtctcgagacgatgactacgagaccatagccatgtctaccatgcatacggatgtgtccaagacgtctctgaagcaggctagcccagtcgcatttaaaaaaatcagtatgaatgacaatgaaaaagtctgaagctgctcagagcatatggtcccaggccatatctgtggaaaaacaagcacagcctgccgcatgctttctttacctatgctctgggggaacggaatgaggcgcgcttggaaagcccaggacatctgtgttaaatttgactgcttttgatggttgccctgattacttagaaatctagattactttgtaatctatctctggcaacagttttgactagatgtcctgtgtgtgtgtgtgtgtgtgtgtgtgtgtgtgtgtgtgtgtgtgtgtgtatgtgtgtgcgcgcgcgcgcgcgcgcacgtgcaaaagagaaagagagagagagaaggagagagacagacagactgcctgtctgtctgtctgtctgtctttctttatgtatgtgtttgaattatgcatatgacaaagagttttacattgtgtttgttggagtgaatttctctgaagtaatgtcatgagctcatttcaaaagcagtcaccacctgatattctcgagaggctgaattttcaagatcagatgagatttccgagaccccggactaccttcgttccctgctaggcatcatcttagtctgtcacaagggtgacagtatacaaagtcacctttttgaatgtgcctgagtcaaaagagtgtctgaagtcatttggcagcatcttttccttttcctctctatttctgtaaggactcaatttcttatac

SEQ ID NO: 12 - protein, 382 aa, mouse neuropeptide Y1 receptor
MNSTLFSKVENHSIHYNASENSPLLAFENDDCHLPLAVIFTLALAYGAVIILGVSGNLALIIIILKQKEMRNVTNILIVNLSFSDLLVAVMCLPFTFVYTLMDHWVFGETMCKLNPFVQCVSITVSIFSLVLIAVERHQLIINPRGWRPNNRHAYIGITVIWVLAVASSLPFVIYQILTDEPFQNVSLAAFKDKYVCFDKFPSDSHRLSYTTLLLVLQYFGPLCFIFICYFKIYIRLKRRNNMMDKIRDSKYRSSETKRINIMLLSIVVAFAVCWLPLTIFNTVFDWNHQIIATCNHNLLFLLCHLTAMISTCVNPIFYGFLNKNFQRDLQFFFNFCDFRSRDDDYETIAMSTMHTDVSKTSLKQASPVAFKKISMNDNEKV

SEQ ID NO: 13 - DNA, 2650 bp, mouse neuropeptide Y2 receptor
  CDS (781)..(1938)
acagtcaacacgcggaaggtggagaaagttgtattggtgaggcttattggttagaaagaccattctagttggtgacttccagaccagtgagaaggcctgtctcaaaaaacaatgtggatggcacctgaggaatgatacctgagggttgcttttgactatagacacagatgttcacactgaaccacccacacccccccccacacacacaccagagggggggggggcttatgtgcacaagaagtgaaagaaaaaagggctgtgtgagaatccagctgttttgccttattttaatctcccaaagatcaggtgctcagcacatgcagatgtttgcccctttgttatattttaggctgctgtatacttacacatttcaaacaattaagtgggtgacactttcctttaagttaatgtgttgacttcatagcaattataaaattgataaactttattgcattcattttgcattaaaatggaatttatttgatctcactcattgtggagctgattctctctctaacaggaaagtgtagttttgtcatgatcatagatattttgaattcctgagttcatccatccctagttgttaatcgacttatgtaaaggatttgcttcatcaagcttttgcaagatctacagtgtgatgaatcagaacacagctatccagagagctcactctaaactaaatcaatccctttagaatggttctctgtttcactaacttttttttaatgtcatttttattatagattcttgtgttatttacaggccaagtgagagtgaatggttctgaagatgggcccggtaggtgcagaggcagatgagaatcaaactgtagaagtgaaagtggagccctatgggccagggcacactactcctagaggtgagttgccccctgatccggagccggagctcatagacagcaccaaactggtcgaggtgcaggtgatcctcatattggcctactgctccatcatcttgctaggggtagttggcaactccctggtaatccatgtggtaatcaaattcaagagcatgcgcacagtaaccaacttttttattgccaacctggctgtggcggatcttttggtgaacaccctgtgcctgccattcactcttacctataccttgatgggagagtggaaaatgggtccggtcttgtgccatttggtgccctatgcccagggtctggcggtacaagtgtccacaataactttgacagtcattgctctggaccgccatcgttgcattgtctaccacctggagagcaagatctccaagcgaatcagcttcctgatcattggcctggcctggggcatcagcgctctgctggcaagtccactggccatcttccgggaatactccctgattgagatcattcctgactttgagattgtggcctgtaccgagaagtggcctggggaagagaagagtgtgtatggtacagtctacagcctttccaccctgctcatcctgtacgttttgcctctgggcatcatatctttctcctacacccgtatctggagtaagctgaggaaccacgtcagtcctggagctgcaagtgaccattaccatcagcgaaggcacaaaatgaccaaaatgctggtgtgcgtggtagtggtgtttgcagtcagctggctacccctccacgccttccaactcgctgtggacatcgacagccacgtcctggacctgaaggagtacaaactcatcttcaccgtgttccacattatcgccatgtgctccacctttgccaacccccttctctatggttggatgaatagcaactacagaaaagctttcctctcggccttccgctgtgagcagaggttggatgccattcactcggaggtgtctatgaccttcaaggctaaaaagaacctggaagtcaaaaagaacaatggccccactgactctttttcggaggctaccaatgtgtaaggacacaggtgtgaaagcacatgggtgaattgtaaccagcgctgccaatctggttagggaaggttttctggccagtgcatgcagacctcccattgtattgactcaaaaagcaacagaaccgaagccccagcagttttatttctggaaaactggctggcagaaggaggtaaaaataaacagattgccatggcacaacgttctgattaccgatgcttggattgtaggttgaattatgagtagagaagggaaggaaaacagaacaaggagttggcaccagcatggttaaaagggagaagtaagtgttatctctgagagcacagtaggatctgcatcaggcacactgtggactccatgggctccctcatcacttgatgaaaagctgctaaacaactcagatttccctagggagctacaggctctctgttagggtgttttggttttattgtctttcctaagatgaaacccaaatgagaatgctataggtaaacatggcttgctacctaaggcggtgacttcaagataacgtcaagagaataaacacatttgctatacgagtaatgttttggcaatgatgggagagattcttatataatcagtgagcaattagttgttgttcagatcaaatgcactactgttgaaagtttgttttttagttcagaatcaaaatctgaaattatcatactgaaaaggaatgaaaacaattgctttattt

SEQ ID NO: 14 - protein, 385 aa, mouse neuropeptide Y2 receptor
MVLKMGPVGAEADENQTVEVKVEPYGPGHTTPRGELPPDPEPELIDSTKLVEVQVILILAYCSIILLGVVGNSLVIHVVIKFKSMRTVTNFFIANLAVADLLVNTLCLPFTLTYTLMGEWKMGPVLCHLVPYAQGLAVQVSTITLTVIALDRHRCIVYHLESKISKRISFLIIGLAWGISALLASPLAIFREYSLIEIIPDFEIVACTEKWPGEEKSVYGTVYSLSTLLILYVLPLGIISFSYTRIWSKLRNHVSPGAASDHYHQRRHKMTKMLVCVVVVFAVSWLPLHAFQLAVDIDSHVLDLKEYKLIFTVFHIIAMCSTFANPLLYGWMNSNYRKAFLSAFRCEQRLDAIHSEVSMTFKAKKNLEVKKNNGPTDSFSEATNV

SEQ ID NO: 15 - DNA, 1709 bp, mouse neuropeptide Y4 receptor
  CDS (204)..(1331)
atgcctgcctcttaccttaagccttgtatgatagcctccaatgccatgagatataagcagccaagaaccttttcccttgcccttatcgttacctggttccattgtagatgcatggtccttgagttccatttgtttgttttgcaggctgcatctctgaagtaggccctttactcctggagttcccggatcttctcacacctaccatgaatacctctcatttcttggcccctctcttcccaggatccctacagggtaagaatgggaccaatccattggattccccctataatttctctgatggctgccaggattcggcagaactgttggccttcatcatcaccacctacagcattgagaccatcttaggggtcctgggaaacctctgcttgatatttgtgaccacaagacaaaaggaaaagtccaatgtgaccaacctactcattgccaacctggccttctctgacttcctcatgtgcctcatctgccaaccactcacagtcacctacaccatcatggattactggatctttggtgaagtcctttgcaagatgttaactttcatccagtgtatgtcagtgacagtctccatcctctcactggtccttgtggccctggagagacaccagctcattatcaatccaacaggctggaaacccagtattttccaggcctacctggggattgtggtcatctggttcatctcttgtttcctttccttgccgttcctggccaacagcaccctgaatgacctcttccactacaaccactctaaggttgtagagtttctggaagacaaggtcgtctgctttgtgtcctggtcttcagatcaccaccgtctcatctataccacctttctgctgctctttcagtactgcatccctctagccttcatcctggtctgctacatacgcatctaccagcgcctgcagaggcagaagcatgtgttccatgcgcacgcttgcagctcacgagcggggcagatgaagcggatcaacagcatgctcatgacaatggtgactgcctttgcagttctctggctacccctgcatgtgttcaacactctggaggactggtaccaggaagccatccctgcttgccatggcaacctcatcttcttgatgtgccacctgttggccatggcttccacctgtgtcaaccctttcatctatggctttctcaacatcaacttcaagaaggatatcaaggctctggtgctgacctgccattgcaggtcacctcaaggggagtctgagcatctgcccctgtccactgttcacacggacctctccaagggatcgatgaggatgggtagcaagtctaacttcatatagttgtgtctgggcttttccctaccattttttttgacacatcctttcacttagttaagaagacacattgcaggctgtgatagcatcctgtcatttctggcttttggggcccagataggttggcaagagacttgaagcttggcattcagatggtttagccctttgcttctgagagatctctgagtcaggattctgcagatcacagagggaactttgtggcttgagctgcaagggtattagagtcagaagtggctgactctcacagccactcagtacagatgcctggcccaaaagccttcatctatgtcctgaccattcagctaacctgcctttggtgatgtgcttatgttcttccaagggatgttgggtgtttcagtatgg

SEQ ID NO: 16 - protein, 375 aa, mouse neuropeptide Y4 receptor
MNTSHFLAPLFPGSLQGKNGTNPLDSPYNFSDGCQDSAELLAFIITTYSIETILGVLGNLCLIFVTTRQKEKSNVTNLLIANLAFSDFLMCLICQPLTVTYTIMDYWIFGEVLCKMLTFIQCMSVTVSILSLVLVALERHQLIINPTGWKPSIFQAYLGIVVIWFISCFLSLPFLANSTLNDLFHYNHSKVVEFLEDKVVCFVSWSSDHHRLIYTTFLLLFQYCIPLAFILVCYIRIYQRLQRQKHVFHAHACSSRAGQMKRINSMLMTMVTAFAVLWLPLHVFNTLEDWYQEAIPACHGNLIFLMCHLLAMASTCVNPFIYGFLNINFKKDIKALVLTCHCRSPQGESEHLPLSTVHTDLSKGSMRMGSKSNFI

SEQ ID NO: 17 - DNA, 1868 bp, mouse neuropeptide Y5 receptor
  CDS (100)..(1500)
tctagatgttagttgtgttctgagtatgttattgtcatagcgtgctattgttcttcaagctgctaatggacactgtcttcttccaagcaggactctagtatggaggttaaacttgaagagcattttaacaagacatttgtcacggagaacaatactgctgccagtcagaacacggcctcccctgcctgggaggactacagaggcacagagaacaatacttctgctgctcggaacactgcctttccagtctgggaggactatagaggcagcgtagacgacttacaatacttcctgattgggctctatacatttgtaagtcttcttggttttatgggaaatctacttatcttaatggctgttatgaaaaagcgcaatcagaagactacagtgaactttctcataggcaacctggccttctccgacattttggttgtcctgttttgctcccctttcaccctgacctctgtcttgttggatcagtggatgttcggcaaagccatgtgccatatcatgccattccttcagtgtgtatcagttctggtttcaactctgattttaatatcgattgccattgtcaggtatcatatgataaagcaccctatatctaacaatttaacagcaaaccatggctacttcctgatagctactgtctggacactgggctttgccatctgttctcccctcccagtgtttcacagccttgtggaacttaaggaaacctttggctcagcattgctaagcagcaagtatttgtgtgttgagtcatggccctctgattcatacagaattgctttcacaatctctttattgttagttcagtatatcctgcctctagtatgtttaacagtaagtcatactagtgtctgcaggagtataagctgtggattgtcccacaaagaaaacagactcgaagaaaatgagatgatcaacttaactctacatccatccaaaaagagtcgggaccaggcaaaaccccccagcactcaaaagtggagctactcattcatcagaaagcaccgaagaaggtacagcaagaagacggcatgcgtgttacccgccccagcaggaccttcccaggagaagcacctaaccgttccagaaaacccaggctcggtccgtagccagctgtcaccatccagtaaggttattccaggggtcccgatctgctttgaggtgaaacctgaagaaagctcagatgctcaggagatgagagtcaagcgttccctcacgagaataaagaagagatctcgcagtgttttctacagactgactatattgatattagtgttcgctgttagctggatgccactccacgtcttccacgtggtgaccgatttcaatgataacctgatttccaataggcatttcaagctggtgtactgcatctgtcacttgttaggcatgatgtcctgttgtcttaatccgatcttatatggattccttaataatggtatcaaagcagacttgagagcccttatccactgcctacacatgtcatgattctctctgtgcaccgaggagagaagaaatgtggagactgcccacaatacatctgtgctaattgatgcataatttacataaacgtgttctggatctgaatgccagtttgtaatctatgtaagatcatttatgttataatgtggttaattccgtcacttgtgcagagtcaatgtcgatctaaggaaatttctgtcttgaaatagttacattaccgtccatttcatgtcattggtaataagttgagtgtcttcggtttcgagtaaaagttatagctatccaaattgttattttgtacaaaaatgtaagaagtgaaaaagttgttccaaagaatatttaacctcagatttaaggaatttcttttatctaga

SEQ ID NO: 18 - protein, 466 aa, mouse neuropeptide Y5 receptor
MEVKLEEHFNKTFVTENNTAASQNTASPAWEDYRGTENNTSAARNTAFPVWEDYRGSVDDLQYFLIGLYTFVSLLGFMGNLLILMAVMKKRNQKTTVNFLIGNLAFSDILVVLFCSPFTLTSVLLDQWMFGKAMCHIMPFLQCVSVLVSTLILISIAIVRYHMIKHPISNNLTANHGYFLIATVWTLGFAICSPLPVFHSLVELKETFGSALLSSKYLCVESWPSDSYRIAFTISLLLVQYILPLVCLTVSHTSVCRSISCGLSHKENRLEENEMINLTLHPSKKSRDQAKPPSTQKWSYSFIRKHRRRYSKKTACVLPAPAGPSQEKHLTVPENPGSVRSQLSPSSKVIPGVPICFEVKPEESSDAQEMRVKRSLTRIKKRSRSVFYRLTILILVFAVSWMPLHVFHVVTDFNDNLISNRHFKLVYCICHLLGMMSCCLNPILYGFLNNGIKADLRALIHCLHMS

SEQ ID NO: 19 - DNA, 2281 bp, mouse neuropeptide y6 receptor
  CDS (823)..(1938)
ctgcagtctattggatgaagagtgtacatattcatataattcttaaagtaggcagaaattaaaggggatggaaatatatacttgtactgccttagatagtcaccaggatgttgttacagtcttcgtttactgcttctgaagcctatactgatagaattaataaaatactgagagagagagagagggacagagagagagagggggagagagagagagagagagagagagagagagagagagagagagagagagaagagaagaaaacaaggtsaagccatctgcttaacttatgtccacattctctcaagagcattgtcctatttgtagaattatctatattgttaagaatcatctccattgttaagattttgtgggctggagatccagctctgttgataaagtgcttgcctaacatgcatgaagtcctaggttctattcccaaggctacataaaaccttgtgttgtgatgaatgcctgtaatcccagtacgcagcaaggagagacaaggaggatcagaagcttaaggacatcattttgtacatagtgagtttgaggaaagctgaggttacatggaactctctctctctcaaaaacaaaacaaaacaaaacaaaaccttctactaatattctggattctgtttgatttttaggatctcaagagcatgctgacgtcatttatgtgtttccatcagatacagacagagatcataaacatttaactcattgattatatgttgagagttgtccctcaagaaccaatggccaaacatccactgaggatacacggaagcttagaaaatctctaattaaaatcctgacataatggaagtgctcacaaaccagccaacacctaataaaaccagtggcaagagcaacaactcggcatttttctactttgaatcctgccaacccccttttctagccatactcttgctactcatagcatatactgtgatcctaatcatgggcatttttggaaacctctctcttatcatcatcatctttaagaaacagagagaagctcaaaatgttaccaacatactgattgccaacctgtccctctctgacatcttggtgtgtgtcatgtgcatcccttttacggtcatctacactctgatggaccactgggtatttgggaacactatgtgtaaactcacttcctacgtgcaaagtgtctcagtttctgtgtccatattctcccttgtgttgattgctattgaacgatatcagctgattgtgaacccccgtggctggaaacccagagtagctcatgcctattgggggatcatcttgatttggctcatttctctgacattgtctattcccttattcctgtcctaccacctcaccaatgagccctttcataatctctctctccctactgacatctacacccaccaggtagcttgtgtggagatttggccttctaaactgaaccaactcctcttttctacatcattatttatgctccagtattttgtccctctgggtttcattcttatctgctacctgaagatcgttctctgcctccgaaaaagaactaggcaggtggacaggagaaaggaaaataagagccgtctcaatgagaacaagagggtaaatgtgatgttgatttccatcgtagtgacttttggagcctgctggttgcccttgaacattttcaatgtcatcttcgactggtatcatgagatgctgatgagctgccaccacgacctggtatttgtagtttgccacttgattgctatggtttctacttgcataaatcctctcttttatggatttctcaacaaaaacttccagaaggatctaatgatgcttattcaccactgttggtgtggtgaacctcaggaaagttatgaaaatattgccatgtctactatgcacacagatgaatccaagggatcattaaaactggctcacataccaacaggcatatagaaactggtaagcaaaatcaaagcccttctgttatgaaagaaagagaagaaatagtatggaatagggcaaggtgcagaggaagccagacttaaacacataatatctttgggcccagttttgctttaagttaagcatgtctactccattcagccatagaacacacagagatttatccctaccctttctttttttcctttggaagaataataacttaaacaacctagacatcattactgaggaagagaacaaaaatgagagagcatacaaggacagcagagatgtctggggtacaaaattcacgttattcgctggaatagctagaaagttattagttgtgctgcag

SEQ ID NO: 20 - protein, 371 aa, mouse neuropeptide y6 receptor
MEVLTNQPTPNKTSGKSNNSAFFYFESCQPPFLAILLLLIAYTVILIMGIFGNLSLIIIIFKKQREAQNVTNILIANLSLSDILVCVMCIPFTVIYTLMDHWVFGNTMCKLTSYVQSVSVSVSIFSLVLIAIERYQLIVNPRGWKPRVAHAYWGIILIWLISLTLSIPLFLSYHLTNEPFHNLSLPTDIYTHQVACVEIWPSKLNQLLFSTSLFMLQYFVPLGFILICYLKIVLCLRKRTRQVDRRKENKSRLNENKRVNVMLISIVVTFGACWLPLNIFNVIFDWYHEMLMSCHHDLVFVVCHLIAMVSTCINPLFYGFLNKNFQKDLMMLIHHCWCGEPQESYENIAMSTMHTDESKGSLKLAHIPTGI